(19) United States
(12) Patent Application Publication
(10) Pub. No.: US 2007/0259377 A1
(43) Pub. Date: Nov. 8, 2007

(54) Diabetes-associated markers and methods of use thereof

(76) Inventors: Mickey Urdea, Alamo, CA (US); Michael McKenna, Berkeley, CA (US); Patrick Arensdorf, Palo Alto, CA (US)

Correspondence Address: MINTZ LEVIN COHN FERRIS GLOVSKY & POPEO, 666 THIRD AVENUE, NEW YORK, NY, 10017, US

(21) Appl. No.: 11/788,260

(22) Filed: Apr. 18, 2007

Related U.S. Application Data
(63) Continuation-in-part of application No. 11/546,874, filed on Oct. 11, 2006.
(60) Provisional application No. 60/725,462, filed on Oct. 11, 2005.

(51) Int. Cl. G01N 33/53

(52) U.S. Cl. 435/7.1

(57) ABSTRACT

Disclosed are methods of identifying subjects with Diabetes, pre-Diabetes, or a pre-diabetic condition, methods of identifying subjects at risk for developing Diabetes, pre-Diabetes, or a pre-diabetic condition, methods of differentially diagnosing diseases associated with Diabetes, pre-Diabetes, or a pre-diabetic condition from other diseases or within sub-classifications of Diabetes, methods of evaluating the risk of progression to Diabetes, pre-Diabetes, or a pre-diabetic condition in patients, methods of evaluating the effectiveness of treatments in subjects with Diabetes, pre-Diabetes, or a pre-diabetic condition, and methods of selecting therapies for treating Diabetes, pre-Diabetes or a pre-diabetic condition, using biomarkers.

### INCORPORATION BY REFERENCE

This application is a continuation-in-part of U.S. patent application Ser. No. 11/546,874, filed on Oct. 11, 2006, which claims priority from U.S. Provisional Application Ser. No. 60/725,462, filed on Oct. 11, 2005.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### FIELD OF THE INVENTION

The present invention relates generally to the identification of biological markers associated with an increased risk of developing Diabetes, as well as methods of using such biological markers in screening, prevention, diagnosis, therapy, monitoring, and prognosis of Diabetes and pre-Diabetes.

### BACKGROUND OF THE INVENTION

Diabetes Mellitus describes a metabolic disorder characterized by chronic hyperglycemia with disturbances of carbohydrate, fat and protein metabolism that result from defects in insulin secretion, insulin action, or both. Diabetes may be present with characteristic symptoms such as thirst, polyuria, blurring of vision, chronic infections, slow wound healing, and weight loss. In its most severe forms, ketoacidosis or a non-ketotic hyperosmolar state may develop and lead to stupor, coma and, in the absence of effective treatment, death. Often symptoms are not severe, not recognized, or may be absent. Consequently, hyperglycemia sufficient to cause pathological and functional changes may be present for a long time, occasionally up to ten years, before a diagnosis is made, usually by the detection of high levels of glucose in urine after overnight fasting during a routine medical work-up. The long-term effects of Diabetes include progressive development of complications such as retinopathy with potential blindness, nephropathy that may lead to renal failure, neuropathy, microvascular changes, and autonomic dysfunction. People with Diabetes are also at increased risk of cardiovascular, peripheral vascular, and cerebrovascular disease (together, “arteriovascular” disease), as well as an increased risk of cancer. Several pathogenetic processes are involved in the development of Diabetes, including processes which destroy the insulin-secreting beta cells of the pancreas with consequent insulin deficiency, and changes in liver and smooth muscle cells that result in the resistance to insulin uptake. The abnormalities of carbohydrate, fat and protein metabolism are due to deficient action of insulin on target tissues resulting from insensitivity to insulin (insulin resistance) or lack of insulin (loss of beta cell function).

Diabetes Mellitus is subdivided into Type 1 Diabetes and Type 2 Diabetes. Type 1 Diabetes results from autoimmune mediated destruction of the beta cells of the pancreas. Individuals with Type 1 Diabetes often become dependent on supplemented insulin for survival and are at risk for ketoacidosis. Patients with Type 1 Diabetes exhibit little or no insulin secretion as manifested by low or undetectable levels of insulin or plasma C-peptide (also known in the art as “soluble C-peptide”).

Type 2 Diabetes is the most common form of Diabetes and is characterized by disorders of insulin action and insulin secretion, either of which may be the predominant feature. Type 2 Diabetes patients are characterized with a relative, rather than absolute, insulin deficiency and are insulin resistant. At least initially, and often throughout their lifetime, these individuals do not need supplemental insulin treatment to survive. Type 2 Diabetes accounts for 90-95% of all cases of Diabetes and can go undiagnosed for many years because the hyperglycemia is often not severe enough to provoke noticeable symptoms of Diabetes or symptoms are simply not recognized. The majority of patients with Type 2 Diabetes are obese, and obesity itself may cause or aggravate insulin resistance. Many of those who are not obese by traditional weight criteria may have an increased percentage of body fat distributed predominantly in the abdominal region (visceral fat). Whereas patients with this form of Diabetes may have insulin levels that appear normal or elevated, the high blood glucose levels in these diabetic patients would be expected to result in even higher insulin values had their beta cell function been normal. Thus, insulin secretion is often defective and insufficient to compensate for the insulin resistance. On the other hand, some hyperglycemic individuals have essentially normal insulin action, but markedly impaired insulin secretion.

Diabetic hyperglycemia can be decreased by weight reduction, increased physical activity, and/or pharmacological treatment. There are several biological mechanisms that are associated with hyperglycemia such as insulin resistance, insulin secretion, and gluconeogenesis, and there are orally active drugs available that act on one or more of these mechanisms. With lifestyle and/or drug intervention, glucose levels can return to near-normal levels, but this is usually temporary. With time, additional second-tier drugs are often required additions to the treatment approach. Multiple agents are available, and combination therapy is common based on failure to maintain glucose or glycosylated hemoglobin (HBA1c) targets. HBA1c is a surrogate measure of the average glucose levels in an individual's blood over the previous few months. Often with time, even these multi-drug approaches fail, at which point insulin injections are instituted.

Over 18 million people in the United States have Type 2 Diabetes, and of these, about 5 million do not know they have the disease. These persons, who do not know they have the disease and who do not exhibit the classic symptoms of Diabetes, present a major diagnostic and therapeutic challenge. Nearly 41 million persons in the United States are at significant risk of developing Type 2 Diabetes. These persons are broadly referred to as “pre-diabetics.” A “pre-diabetic” or a subject with pre-Diabetes represents any person or population with a significantly greater risk than the broad population for conversion to Type 2 Diabetes in a given period of time. The risk of developing Type 2 Diabetes increases with age, obesity, and lack of physical activity. It occurs more frequently in women with prior gestational Diabetes, and in individuals with hypertension and/or dyslipidemia. Its frequency varies in different ethnic subgroups. Type 2 Diabetes is often associated with familial, likely genetic, predisposition, however the genetics of this form of Diabetes are complex and not clearly defined.

Pre-diabetics often have fasting glucose levels between normal and frank diabetic levels. Abnormal glucose tolerance, or “impaired glucose tolerance” can be an indication that an individual is on the path toward Diabetes; it requires the use of a 2-hour oral glucose tolerance test for its detection. However, it has been shown that impaired glucose tolerance is by itself entirely asymptomatic and unassociated with any functional disability. Indeed, insulin secretion is typically greater in response to a mixed meal than in response to a pure glucose load; as a result, most persons with impaired glucose tolerance are rarely, if ever, hyperglycemic in their daily lives, except when they undergo diagnostic glucose tolerance tests. Thus, the importance of impaired glucose tolerance resides exclusively in its ability to identify persons at increased risk of future disease (Stem et al, 2002). In studies conducted by Stem and others, the sensitivity and false-positive rates of impaired glucose tolerance as a predictor of future conversion to Type 2 Diabetes was 50.9% and 10.2%, respectively, representing an area under the Receiver-Operating Characteristic Curve of 77.5% (95% confidence interval (CI) of 74.3-80.7%) and a P-value (Hosmer-Lemeshow goodness-of-fit) of 0.20. Because of its cost, reliability, and inconvenience, the oral glucose tolerance test is seldom used in routine clinical practice. Moreover, patients whose Diabetes is diagnosed solely on the basis of an oral glucose tolerance test have a high rate of reversion to normal on follow-up and may in fact represent false-positive diagnoses. Stem and others reported that such cases were almost 5 times more likely to revert to non-diabetic status after 7 to 8 years of follow-up compared with persons meeting conventional fasting or clinical diagnostic criteria.

Beyond glucose and HBA1c, several single time point biomarker measurements have been attempted for the use of risk assessment for future Diabetes. U.S. Patent Application No. 2003/0100486 proposes C-Reactive Protein (CRP) and Interleukin-6 (IL-6), both markers of systemic inflammation, used alone and as an adjunct to the measurement of HBA1c. However, for practical reasons relating to clinical performance, specifically poor specificity and high false positive rates, these tests have not been adopted.

Often a person with impaired glucose tolerance will be found to have at least one or more of the common arteriovascular disease risk factors (e.g., dyslipidemia and hypertension). This clustering has been termed “Syndrome X,” or “Metabolic Syndrome” by some researchers and can be indicative of a diabetic or pre-diabetic condition. Alone, each component of the cluster conveys increased arteriovascular and diabetic disease risk, but together as a combination they become much more significant. This means that the management of persons with hyperglycemia and other features of Metabolic Syndrome should focus not only on blood glucose control but also include strategies for reduction of other arteriovascular disease risk factors. Furthermore, such risk factors are non-specific for Diabetes or pre-Diabetes and are not in themselves a basis for a diagnosis of Diabetes, or of diabetic status.

It should furthermore be noted that an increased risk of conversion to Diabetes implies an increased risk of converting to arteriovascular disease and events. Diabetes itself is one of the most significant single risk factors for arteriovascular disease, and is in fact often termed a “coronary heart disease equivalent” by itself, indicating a greater than 20 percent ten-year risk of an arteriovascular event, in a similar risk range with stable angina and just below the most significant independent risk factors, such as survivorship of a previous arteriovascular event. Diabetes is also a major risk factor for other arteriovascular disease, such as peripheral artery disease or cerebrovascular disease.

Risk prediction for Diabetes, pre-Diabetes, or a pre-diabetic condition can also encompass multi-variate risk prediction algorithms and computed indices that assess and estimate a subject's absolute risk for developing Diabetes, pre-Diabetes, or a pre-diabetic condition with reference to a historical cohort. Risk assessment using such predictive mathematical algorithms and computed indices has increasingly been incorporated into guidelines for diagnostic testing and treatment, and encompass indices obtained from and validated with, inter alia, multi-stage, stratified samples from a representative population. A plurality of conventional Diabetes risk factors is incorporated into predictive models. A notable example of such algorithms include the Framingham study (Kannel, W. B. et al, (1976) Am. J. Cardiol. 38: 46-51) and modifications of the Framingham Study, such as the National Cholesterol Education Program Expert Panel on Detection, Evaluation, and Treatment of High Blood Cholesterol in Adults (Adult Treatment Panel III), also known as NCEP/ATP III, which incorporates a patient's age, total cholesterol concentration, HDL cholesterol concentration, smoking status, and systolic blood pressure to estimate a person's 10-year risk of developing arteriovascular disease, which is commonly found in subjects suffering from or at risk for developing Diabetes Mellitus, or a pre-diabetic condition. The same Framingham algorithm has been found to be modestly predictive of the risk for developing Diabetes Mellitus, or a pre-diabetic condition.

Other Diabetes risk prediction algorithms include, without limitation, the San Antonio Heart Study (Stem, M. P. et al, (1984) Am. J. Epidemiol. 120: 834-851; Stern, M. P. et al, (1993) Diabetes 42: 706-714; Burke, J. P. et al, (1999) Arch. Intern. Med. 159: 1450-1456), Archimedes (Eddy, D. M. and Schlessinger, L. (2003) Diabetes Care 26(11): 3093-3101; Eddy, D. M. and Schlessinger, L. (2003) Diabetes Care 26(11): 3102-3110), the Finnish-based Diabetes Risk Score (Lindström, J. and Tuomilehto, J. (2003) Diabetes Care 26(3): 725-731), and the Ely Study (Griffin, S. J. et al, (2000) Diabetes Metab. Res. Rev. 16: 164-171), the contents of which are expressly incorporated herein by reference.

Despite the numerous studies and algorithms that have been used to assess the risk of Diabetes, pre-Diabetes, or a pre-diabetic condition, the evidence-based, multiple risk factor assessment approach is only moderately accurate for the prediction of short- and long-term risk of manifesting Diabetes, pre-Diabetes, or a pre-diabetic condition in individual asymptomatic or otherwise healthy subjects. Furthermore, due to issues of practicality and the difficulty of the risk computations involved, there has been little adoption of such an approach by the primary care physician that is most likely to initially encounter the pre-diabetic or undiagnosed early diabetic. Clearly, there remains a need for improved methods of assessing the risk of future Diabetes.

It is well documented that pre-Diabetes can be present for ten or more years before the detection of glycemic disorders like Diabetes. Treatment of pre-diabetics with drugs such as acarbose, metformin, troglitazone and rosiglitazone can postpone or prevent Diabetes; yet few pre-diabetics are treated. A major reason, as indicated above, is that no simple and unambiguous laboratory test exists to determine the actual risk of an individual to develop Diabetes. Furthermore, even in individuals known to be at risk of Diabetes, glycemic control remains the primary therapeutic monitoring endpoint, and is subject to the same limitations as its use in the prediction and diagnosis of frank Diabetes. Thus, there remains a need in the art for methods of identifying, diagnosing, and treatment of these individuals who are not yet diabetics, but who are at significant risk of developing Diabetes.

### SUMMARY OF THE INVENTION

The present invention relates in part to the discovery that certain biological markers (referred to herein as “biomarkers”), such as proteins, nucleic acids, polymorphisms, metabolites, and other analytes, as well as certain physiological conditions and states, are present or altered in subjects with an increased risk of developing Diabetes, pre-Diabetes, or a pre-diabetic condition such as, but not limited to, Metabolic Syndrome (Syndrome X), conditions characterized by impaired glucose regulation and/or insulin resistance, such as Impaired Glucose Tolerance (IGT) and Impaired Fasting Glycemia (IFG), but where such subjects do not exhibit some or all of the conventional risk factors of these conditions, or subjects who are asymptomatic for Diabetes, pre-Diabetes, or a pre-diabetic condition.

Accordingly, the invention provides biomarkers of Diabetes, pre-Diabetes, or pre-diabetic conditions that, when used together in combinations of three or more such biomarker combinations, or “panels,” can be used to assess the risk of subjects experiencing such Diabetes, pre-Diabetes, or pre-diabetic conditions, to diagnose or identify subjects with Diabetes, pre-Diabetes, or a pre-diabetic condition, to monitor the risk for development of Diabetes, pre-Diabetes, or a pre-diabetic condition, to monitor subjects that are undergoing therapies for Diabetes, pre-Diabetes, or a pre-diabetic condition, to differentially diagnose disease states associated with Diabetes or a pre-diabetic condition from other diseases, or within sub-classifications of Diabetes, pre-Diabetes, or pre-diabetic conditions, to evaluate changes in the risk of Diabetes, pre-Diabetes, or pre-diabetic conditions, and to select or modify therapies or interventions for use in treating subjects with Diabetes, pre-Diabetes, or a pre-diabetic condition, or for use in treating subjects who are at risk for developing Diabetes, pre-Diabetes, or a pre-diabetic condition. Preferably, the present invention provides use of a panel of biological markers, some of which are unrelated to Diabetes or have not heretofore been identified as related to Diabetes, but are related to early biological changes that can lead to the development of Diabetes, pre-Diabetes, or a pre-diabetic condition, to detect and identify subjects who exhibit none of the symptoms for Diabetes, i.e., who are asymptomatic for Diabetes, pre-Diabetes, or pre-diabetic conditions or have only non-specific indicators of potential pre-diabetic conditions, such as arteriovascular risk factors, or who exhibit none or few of the conventional risk factor of Diabetes, yet are at risk. Significantly, many of the individual biomarkers disclosed herein have shown little individual significance in the diagnosis of Diabetes, pre-diabetes, or a pre-diabetic condition, but when used in combination with other disclosed biomarkers and combined with the herein disclosed mathematical classification algorithms, traditional laboratory risk factors of Diabetes, and other clinical parameters of Diabetes, becomes significant discriminates of the pre-Diabetes subject from one who is not pre-diabetic or is not at significant risk of developing Diabetes, pre-Diabetes, or a pre-diabetic condition. The methods of the present invention provide an improvement over currently available methods of risk evaluation of the development of Diabetes, pre-Diabetes, or a pre-diabetic condition in a subject by measurement of the biomarkers defined herein.

In particular, the invention relates to the use of three or more such biomarkers from a given subject, with two or more of such biomarkers being T2DMARKERS measured in samples from the subject, chosen from a set including adiponectin (ADIPOQ), C-reactive protein (CRP), fibrinogen alpha chain (FGA), leptin (LEP), insulin (together with its precursors pro-insulin and soluble C-peptide (sCP or SCp); these three variants, used either individually or jointly together, are referred to here as INS or “Insulin”), advanced glycosylation end product-specific receptor (AGER aka RAGE), alpha-2-HS-glycoprotein (AHSG), angiogenin (ANG), apolipoprotein E (APOE), CD14 molecule (CD14), vascular endothelial growth factor (VEGF), ferritin (FTH1), insulin-like growth factor binding protein 1 (IGFBP1), interleukin 2 receptor, alpha (IL2RA), vascular cell adhesion molecule 1 (VCAM1) and Von Willebrand factor (VWF), and a third biomarker measurement optionally chosen from any of the subject's clinical parameters, traditional laboratory risk factors (including, without limitation, glucose, glycosylated hemoglobin (HBA1c), and triglycerides (TRIG)) or other biomarkers, identified herein, in the subject's sample. These three or more biomarkers are combined together by a mathematical process or formula into a single number reflecting the subject's risk for developing Diabetes, pre-Diabetes, or a pre-diabetic condition, or for use in selecting, tailoring, and monitoring effectiveness of various therapeutic interventions, such as treatment of subjects with diabetes-modulating drugs, for said conditions. Additional biomarkers beyond the initial aforementioned three may also be added to the panel from any of T2DMARKERS, clinical parameters, and traditional laboratory risk factors.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and are encompassed by the following detailed description and claims.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 is a table containing key biomarkers, including clinical parameters, traditional laboratory risk factors, and together with core and additional biomarkers, that are used in the predictive models according to the present invention.

FIG. 2 is a graph depicting the Receiver Operator Characteristic (ROC) curve of a Linear Discriminant Analysis (LDA) classification model derived solely from the Clinical Parameters (and excluding the use of any blood-borne biomarkers of the present invention), as measured and calculated for the Base Population of Example 1, and including Area Under the Curve (AUC) and cross-validation statistics using Leave One Out (LOO) and 10-Fold methods.

FIG. 3 is a graph showing a representative clinical global risk assessment index according to the Stem model of Diabetes risk, as measured and calculated for the Base Population of Example 1.

FIG. 4 is a table showing the results of univariate analysis of parameter variances, biomarker transformations, and biomarker mean back-transformed concentration values as measured for both the Case (Converter to Diabetes) and Control (Non-Converter to Diabetes) arm of the Base Population of Example 1.

FIG. 5 is a table summarizing the results of cross-correlation analysis of clinical parameters and biomarkers of the present invention, as measured in the Base Population of Example 1.

FIG. 6A is a graphical tree representation of the results of hierarchical clustering and Principal Component Analysis (PCA) of clinical parameters and biomarkers of the present invention, as measured in the Base Population of Example 1.

FIG. 6B is a bar graph representing the results of hierarchical clustering and PCA of clinical parameters and biomarkers of the present invention, as measured in the Base Population of Example 1.

FIG. 6C is a scatter plot of the results of hierarchical clustering and PCA of clinical parameters and biomarkers of the present invention, as measured in the Base Population of Example 1.

FIG. 7 is a table summarizing the characteristics considered in various predictive models and model types of the present invention, using various model parameters, as measured in the Base Population of Example 1.

FIG. 8 is a graphical representative of the ROC curves for the leading univariate, bivariate, and trivariate LDA models by AUC, as measured and calculated in the Base Population of Example 1. The legend AUC represents the mean AUC of 10-Fold cross-validations for each model, with error bars indicating the standard deviation of the AUCs.

FIG. 9 is a graphical representation of the ROC curves for the LDA stepwise selection model, as measured and calculated in the Base Population of Example 1, using the same format as in FIG. 8.

FIG. 10 is a graph showing the entire LDA forward-selected set of all tested biomarkers with model AUC and Akaike Information Criterion (AIC) statistics at each biomarker addition step, as measured and calculated in the Base Population of Example 1.

FIG. 11 are tables showing univariate ANOVA analysis of parameter variances including biomarker transformation and biomarker mean back-transformed concentration values across non-converters, converters, and diabetics arms, as measured and calculated at baseline in the Total Population of Example 2.

FIG. 12 is a table summarizing the cross-correlation of clinical parameters and biomarkers of the present invention, as measured in the Total Population of Example 2.

FIG. 13 is a graph showing the entire LDA forward-selected set of tested parameters with model AUC and AIC statistics at each biomarker addition step as measured and calculated in the Total Population of Example 2.

FIG. 14 is a graph showing LDA forward-selected set of blood parameters (excluding clinical parameters) alone with model characteristics at each biomarker addition step as measured and calculated in the Total Population of Example 2.

FIG. 15 is a table showing the representation of all parameters tested in Example 1 and Example 2 and according to the T2DMARKER biomarker categories used in the invention.

FIG. 16A and 16B are tables showing biomarker selection under various scenarios of classification model types and Base and Total Populations of Example 1 and Example 2, respectively.

FIG. 17 are tables showing the complete enumeration of fitted LDA models for all potential univariate, bivariate, and trivariate combinations as measured and calculated in for both Total and Base Populations in Example 1 and Example 2, and encompassing all 53 and 49 biomarkers recorded, respectively, for each study as potential model parameters.

FIG. 18 is a graph showing the number and percentage of the total univariate, bivariate, and trivariate models of FIG. 17 which meet various AUC hurdles using the Total Population of Example 1.

### DETAILED DESCRIPTION OF THE INVENTION

The present invention relates to the identification of biomarkers associated with subjects having Diabetes, pre-Diabetes, or a pre-diabetic condition, or who are pre-disposed to developing Diabetes, pre-Diabetes, or a pre-diabetic condition. Accordingly, the present invention features methods for identifying subjects who are at risk of developing Diabetes, pre-Diabetes, or a pre-diabetic condition, including those subjects who are asymptomatic for Diabetes, pre-Diabetes, or a pre-diabetic condition by detection of the biomarkers disclosed herein. These biomarkers are also useful for monitoring subjects undergoing treatments and therapies for Diabetes, pre-Diabetes, or pre-diabetic conditions, and for selecting or modifying therapies and treatments that would be efficacious in subjects having Diabetes, pre-Diabetes, or a pre-diabetic condition, wherein selection and use of such treatments and therapies slow the progression of Diabetes, pre-Diabetes, or pre-diabetic conditions, or prevent their onset.

Definitions

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as “clinical parameters” defined herein, as well as “traditional laboratory risk factors”, also defined herein. Biomarkers also include any calculated indices created mathematically or combinations of any one or more of the foregoing measurements, including temporal trends and differences. The term “analyte” as used herein can mean any substance to be measured and can encompass electrolytes and elements, such as calcium.

“Clinical parameters” encompasses all non-sample or non-analyte biomarkers of subject health status or other characteristics, such as, without limitation, age (AGE), ethnicity (RACE), gender (SEX), diastolic blood pressure (DBP) and systolic blood pressure (SBP), family history (FHX), height (HT), weight (WT), waist (Waist) and hip (Hip) circumference, body-mass index (BMI), past Gestational Diabetes Mellitus (GDM), and resting heart rate.

“T2DMARKER” or “T2DMARKERS” encompass one or more of all biomarkers whose levels are changed in subjects who have Diabetes, pre-Diabetes, or a pre-diabetic condition, or who are at risk for developing Diabetes, pre-Diabetes, or a pre-diabetic condition.

Individual analyte-based T2DMARKERS are summarized in Table 1 below and are collectively referred to herein as, inter alia, “Diabetes risk-associated proteins”, “T2DMARKER polypeptides”, or “T2DMARKER proteins”. The corresponding nucleic acids encoding the polypeptides are referred to as “Diabetes risk-associated nucleic acids”, “Diabetes risk-associated genes”, “T2DMARKER nucleic acids”, or “T2DMARKER genes”. Unless indicated otherwise, “T2DMARKER”, “Diabetes risk-associated proteins”, “Diabetes risk-associated nucleic acids” are meant to refer to any of the sequences disclosed herein. The corresponding metabolites of the T2DMARKER proteins or nucleic acids can also be measured, as well as any of the traditional laboratory risk factors and metabolites previously disclosed, and including, without limitation, such metabolites as dehydroepiandrosterone sulfate (DHEAS); c-peptide; cortisol; vitamin D3; 5-hydroxytryptamine (5-HT; serotonin); oxyntomodulin; estrogen; estradiol; and digitalis-like factor, herein referred to as “T2DMARKER metabolites”.

Non-analyte physiological markers of health status (e.g., such as age, ethnicity, diastolic or systolic blood pressure, body-mass index, and other non-analyte measurements commonly used as conventional risk factors) are referred to as “T2DMARKER physiology”. Calculated indices created from mathematically combining measurements of one or more, preferably two or more of the aforementioned classes of T2DMARKERS are referred to as “T2DMARKER indices”.

“Diabetic condition” in the context of the present invention comprises type I and type II Diabetes Mellitus, and pre-Diabetes (defined herein).

“Diabetes Mellitus” in the context of the present invention encompasses Type 1 Diabetes, both autoimmune and idiopathic and Type 2 Diabetes (referred to herein as “Diabetes” or “T2DM”). The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level≧11.1 mmol/L (≧200 mg/dL). Other values suggestive of or indicating high risk for Diabetes Mellitus include elevated arterial pressure ≧140/90 mm Hg; elevated plasma triglycerides (≧1.7 mmol/L; 150 mg/dL) and/or low HDL-cholesterol (<0.9 mmol/L, 35 mg/dl for men; <1.0 mmol/L, 39 mg/dL women); central obesity (males: waist to hip ratio >0.90; females: waist to hip ratio >0.85) and/or body mass index exceeding 30 kg/m²; microalbuminuria, where the urinary albumin excretion rate ≧20 μg/min or albumin:creatinine ratio ≧30 mg/g).

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining T2DMARKERS and other biomarkers are linear and non-linear equations and statistical classification analyses to determine the relationship between levels of T2DMARKERS detected in a subject sample and the subject's risk of Diabetes. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shruken Centroids (SC), StepAIC, Kth-Nearest Neighbor, Boosting, Decision Trees, Neural Networks, Bayesian Networks, Support Vector Machines, and Hidden Markov Models, among others. Many of these techniques are useful either combined with a T2DMARKER selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Leave-One-Out (LOO) and 10-Fold cross-validation (10-Fold CV).

A “Health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (aka zero predicted subject outcome misclassifications or FP and FN), which all performance measures will favor over imperfection, but to differing degrees.

“Impaired glucose tolerance” (IGT) is a pre-diabetic condition defined as having a blood glucose level that is higher than normal, but not high enough to be classified as Diabetes Mellitus. A subject with IGT will have two-hour glucose levels of 140 to 199 mg/dL (7.8 to 11.0 mmol) on the 75-g oral glucose tolerance test. These glucose levels are above normal but below the level that is diagnostic for Diabetes. Subjects with impaired glucose tolerance or impaired fasting glucose have a significant risk of developing Diabetes and thus are an important target group for primary prevention.

“Insulin resistance” refers to a diabetic or pre-diabetic condition in which the cells of the body become resistant to the effects of insulin, that is, the normal response to a given amount of insulin is reduced. As a result, higher levels of insulin are needed in order for insulin to exert its effects.

“Measuring” or “measurement” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W. B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. In this last, multiple methods are frequently used to defining abnormal or disease values, including reference limits, discrimination limits, and risk thresholds as per Vasan, “Biomarkers of Cardiovascular Disease: Molecular Basis and Practical Considerations,” Circulation 2006, 113: 2335-2362.

Analytical accuracy refers to the repeatability and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Normal glucose levels” is used interchangeably with the term “normoglycemic” and “normal” and refers to a fasting venous plasma glucose concentration of less than 6.1 mmol/L (110 mg/dL). Although this amount is arbitrary, such values have been observed in subjects with proven normal glucose tolerance, although some may have IGT as measured by oral glucose tolerance test (OGTT). Glucose levels above normoglycemic are considered a pre-diabetic condition.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test.

“Positive predictive value” or “PPV” is calculated by TP/(TP+FP) or the true positive fraction of all positive test results. It is inherently impacted by the prevalence of the disease and pre-test probability of the population intended to be tested.

“Pre-Diabetes” or “pre-Diabetic,” in the context of the present invention indicates the physiological state, in an individual or in a population, and absent any therapeutic intervention (diet, exercise, pharmaceutical, or otherwise) of having a higher than normal expected rate of disease conversion to frank Type 2 Diabetes Mellitus. Pre-Diabetes can also refer to those subjects or individuals, or a population of subjects or individuals who will, or are predicted to convert to frank Type 2 Diabetes Mellitus within a given time period or time horizon at a higher rate than that of the general, unselected population. Such absolute predicted rate of conversion to frank Type 2 Diabetes Mellitus in pre-Diabetes populations may be as low as 1 percent or more per annum, but preferably 2 percent per annum or more. It may also be stated in terms of a relative risk from normal between quartiles of risk or as a likelihood ratio between differing biomarker and index scores, including those coming from the invention. Unless otherwise noted, and without limitation, when a categorical positive diagnosis of pre-Diabetes is stated here, it is defined experimentally with reference to the group of subjects with a predicted conversion rate to Type 2 Diabetes Mellitus of two percent (2%) or greater per annum over the coming 5.0 years, or ten percent (10%) or greater in the entire period, of those testing at a given threshold value (the selected pre-Diabetes clinical cutoff). When a continuous measure of Diabetes conversion risk is produced, pre-Diabetes encompasses any expected annual rate of conversion above that seen in a normal reference or general unselected normal prevalence population. When a complete study is retrospectively discussed in the Examples, pre-Diabetes encompasses the baseline condition of all of the “Converters” or “Cases” arms, each of whom converted to Type 2 Diabetes Mellitus during the study.

In an unselected individual population, pre-Diabetes overlaps with, but is not necessarily a complete superset of, or contained subset within, all those with “pre-diabetic conditions;” as many who will convert to Diabetes in a given time horizon are now apparently healthy, and with no obvious pre-diabetic condition, and many have pre-diabetic conditions but will not convert in a given time horizon; such is the diagnostic gap and need to be fulfilled by the invention. Taken as a population, individuals with pre-Diabetes have a predictable risk of conversion to Diabetes (absent therapeutic intervention) compared to individuals without pre-Diabetes and otherwise risk matched.

“Pre-diabetic condition” refers to a metabolic state that is intermediate between normal glucose homeostasis and metabolism and states seen in frank Diabetes Mellitus. Pre-diabetic conditions include, without limitation, Metabolic Syndrome (“Syndrome X”), Impaired Glucose Tolerance (IGT), and Impaired Fasting Glycemia (IFG). IGT refers to post-prandial abnormalities of glucose regulation, while IFG refers to abnormalities that are measured in a fasting state. The World Health Organization defines values for IFG as a fasting plasma glucose concentration of 6.1 mmol/L (100 mg/dL) or greater (whole blood 5.6 mmol/L; 100 mg/dL), but less than 7.0 mmol/L (126 mg/dL)(whole blood 6.1 mmol/L; 110 mg/dL). Metabolic syndrome according to the National Cholesterol Education Program (NCEP) criteria are defined as having at least three of the following: blood pressure ≧130/85 mm Hg; fasting plasma glucose ≧6.1 mmol/L; waist circumference >102 cm (men) or >88 cm (women); triglycerides ≧1.7 mmol/L; and HDL cholesterol <1.0 mmol/L (men) or 1.3 mmol/L (women). Many individuals with pre-diabetic conditions will not convert to T2DM.

“Risk” in the context of the present invention, relates to the probability that an event will occur over a specific time period, as in the conversion to frank Diabetes, and can can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. Alternative continuous measures which may be assessed in the context of the present invention include time to Diabetes conversion and therapeutic Diabetes conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normoglycemic condition to a pre-diabetic condition or pre-Diabetes, or from a pre-diabetic condition to pre-Diabetes or Diabetes. Risk evaluation can also comprise prediction of future glucose, HBA1c scores or other indices of Diabetes, either in absolute or relative terms in reference to a previously measured population. The methods of the present invention may be used to make continuous or categorical measurements of the risk of conversion to Type 2 Diabetes, thus diagnosing and defining the risk spectrum of a category of subjects defined as pre-Diabetic. In the categorical scenario, the invention can be used to discriminate between normal and pre-Diabetes subject cohorts. In other embodiments, the present invention may be used so as to discriminate pre-Diabetes from Diabetes, or Diabetes from normal. Such differing use may require different T2DMARKER combinations in individual panel, mathematical algorithm, and/or cut-off points, but be subject to the same aforementioned measurements of accuracy for the intended use.

A “sample” in the context of the present invention is a biological sample isolated from a subject and can include, by way of example and not limitation, whole blood, serum, plasma, blood cells, endothelial cells, tissue biopsies, lymphatic fluid, ascites fluid, interstitital fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, or any other secretion, excretion, or other bodily fluids.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of Diabetes Mellitus, pre-Diabetes, or pre-diabetic conditions. A subject can be male or female. A subject can be one who has been previously diagnosed or identified as having Diabetes, pre-Diabetes, or a pre-diabetic condition, and optionally has already undergone, or is undergoing, a therapeutic intervention for the Diabetes, pre-Diabetes, or pre-diabetic condition. Alternatively, a subject can also be one who has not been previously diagnosed as having Diabetes, pre-Diabetes, or a pre-diabetic condition. For example, a subject can be one who exhibits one or more risk factors for Diabetes, pre-Diabetes, or a pre-diabetic condition, or a subject who does not exhibit Diabetes risk factors, or a subject who is asymptomatic for Diabetes, pre-Diabetes, or pre-diabetic conditions. A subject can also be one who is suffering from or at risk of developing Diabetes, pre-Diabetes, or a pre-diabetic condition.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms, such as Stem, Framingham, Finland Diabetes Risk Score, ARIC Diabetes, and Archimedes. Traditional laboratory risk factors commonly tested from subject blood samples include, but are not limited to, total cholesterol (CHOL), LDL (LDL/LDLC), HDL (HDL/HDLC), VLDL (VLDLC), triglycerides (TRIG), glucose (including, without limitation, the fasting plasma glucose (Glucose) and the oral glucose tolerance test (OGTT)) and HBA1c (HBA1C) levels.

Diagnostic and Prognostic Indications of the Invention

The invention allows the diagnosis and prognosis of Diabetes, pre-Diabetes, or a pre-diabetic condition. The risk of developing Diabetes, pre-Diabetes, or a pre-diabetic condition can be detected with a pre-determined level of predictability by measuring an “effective amount” of T2DMARKER proteins, nucleic acids, polymorphisms, metabolites, and other analytes in a test sample (e.g., a subject derived sample), and comparing the effective amounts to reference or index values, often utilizing mathematical algorithms or formula in order to combine information from results of multiple individual T2DMARKERS and from non-analyte clinical parameters into a single measurement or index. Subjects identified as having an increased risk of Diabetes, pre-Diabetes, or a pre-diabetic condition can optionally be selected to receive treatment regimens, such as administration of prophylactic or therapeutic compounds such as “Diabetes-modulating agents” as defined herein, or implementation of exercise regimens or dietary supplements to prevent or delay the onset of Diabetes, pre-Diabetes, or a pre-diabetic condition.

The amount of the T2DMARKER protein, nucleic acid, polymorphism, metabolite, or other analyte can be measured in a test sample and compared to the “normal control level”, utilizing techniques such as reference limits, discrimination limits, or risk defining thresholds to define cutoff points and abnormal values for Diabetes, pre-Diabetes, and pre-diabetic conditions, all as described in Vasan, 2006. The normal control level means the level of one or more T2DMARKERS or combined T2DMARKER indices typically found in a subject not suffering from Diabetes, pre-Diabetes, or a pre-diabetic condition. Such normal control level and cutoff points may vary based on whether a T2DMARKER is used alone or in a formula combining with other T2DMARKERS into an index. Alternatively, the normal control level can be a database of T2DMARKER patterns from previously tested subjects who did not convert to Diabetes over a clinically relevant time horizon.

The present invention may be used to make continuous or categorical measurements of the risk of conversion to Type 2 Diabetes, thus diagnosing and defining the risk spectrum of a category of subjects defined as pre-Diabetic. In the categorical scenario, the methods of the present invention can be used to discriminate between normal and pre-Diabetes subject cohorts. In other embodiments, the present invention may be used so as to discriminate pre-Diabetes from Diabetes, or Diabetes from normal. Such differing use may require different T2DMARKER combinations in individual panel, mathematical algorithm, and/or cut-off points, but be subject to the same aforementioned measurements of accuracy for the intended use.

Identifying the pre-Diabetic subject enables the selection and initiation of various therapeutic interventions or treatment regimens in order to delay, reduce or prevent that subject's conversion to a frank Diabetes disease state. Levels of an effective amount of T2DMARKER proteins, nucleic acids, polymorphisms, metabolites, or other analytes also allows for the course of treatment of Diabetes, pre-Diabetes or a pre-diabetic condition to be monitored. In this method, a biological sample can be provided from a subject undergoing treatment regimens, e.g., drug treatments, for Diabetes. Such treatment regimens can include, but are not limited to, exercise regimens, dietary supplementation, bariatric surgical intervention, and treatment with therapeutics or prophylactics used in subjects diagnosed or identified with Diabetes, pre-Diabetes, or a pre-diabetic condition. If desired, biological samples are obtained from the subject at various time points before, during, or after treatment.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progession to conditions like Diabetes, pre-Diabetes, or a pre-diabetic condition, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No.; U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein. Thus, in a health-related data management system, wherein risk of developing a diabetic condition for a subject or a population comprises analyzing Diabetes risk factors, the present invention provides an improvement comprising use of a data array encompassing the biomarker measurements as defined herein and/or the resulting evaluation of risk from those biomarker measurements.

A machine-readable storage medium can comprise a data storage material encoded with machine readable data or data arrays which, when using a machine programmed with instructions for using said data, is capable of use for a variety of purposes, such as, without limitation, subject information relating to Diabetes risk factors over time or in response to diabetes-modulating drug therapies, drug discovery, and the like. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein. Levels of an effective amount of T2DMARKER proteins, nucleic acids, polymorphisms, metabolites, or other analytes can then be determined and compared to a reference value, e.g. a control subject or population whose diabetic state is known or an index value or baseline value. The reference sample or index value or baseline value may be taken or derived from one or more subjects who have been exposed to the treatment, or may be taken or derived from one or more subjects who are at low risk of developing Diabetes, pre-Diabetes, or a pre-diabetic condition, or may be taken or derived from subjects who have shown improvements in Diabetes risk factors (such as clinical parameters or traditional laboratory risk factors as defined herein) as a result of exposure to treatment. Alternatively, the reference sample or index value or baseline value may be taken or derived from one or more subjects who have not been exposed to the treatment. For example, samples may be collected from subjects who have received initial treatment for Diabetes, pre-Diabetes, or a pre-diabetic condition and subsequent treatment for Diabetes, pre-Diabetes, or a pre-diabetic condition to monitor the progress of the treatment. A reference value can also comprise a value derived from risk prediction algorithms or computed indices from population studies such as those disclosed herein.

The T2DMARKERS of the present invention can thus be used to generate a “reference T2DMARKER profile” of those subjects who do not have Diabetes, pre-Diabetes, or a pre-diabetic condition such as impaired glucose tolerance, and would not be expected to develop Diabetes, pre-Diabetes, or a pre-diabetic condition. The T2DMARKERS disclosed herein can also be used to generate a “subject T2DMARKER profile” taken from subjects who have Diabetes, pre-Diabetes, or a pre-diabetic condition like impaired glucose tolerance. The subject T2DMARKER profiles can be compared to a reference T2DMARKER profile to diagnose or identify subjects at risk for developing Diabetes, pre-Diabetes or a pre-diabetic condition, to monitor the progression of disease, as well as the rate of progression of disease, and to monitor the effectiveness of Diabetes, pre-Diabetes or pre-diabetic condition treatment modalities. The reference and subject T2DMARKER profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other Diabetes-risk algorithms and computed indices such as those described herein.

Differences in the genetic makeup of subjects can result in differences in their relative abilities to metabolize various drugs, which may modulate the symptoms or risk factors of Diabetes, pre-Diabetes or a pre-diabetic condition. Subjects that have Diabetes, pre-Diabetes, or a pre-diabetic condition, or at risk for developing Diabetes, pre-Diabetes, or a pre-diabetic condition can vary in age, ethnicity, body mass index (BMI), total cholesterol levels, blood glucose levels, blood pressure, LDL and HDL levels, and other parameters. Accordingly, use of the T2DMARKERS disclosed herein, both alone and together in combination with known genetic factors for drug metabolism, allow for a pre-determined level of predictability that a putative therapeutic or prophylactic to be tested in a selected subject will be suitable for treating or preventing Diabetes, pre-Diabetes, or a pre-diabetic condition in the subject.

To identify therapeutics or drugs that are appropriate for a specific subject, a test sample from the subject can also be exposed to a therapeutic agent or a drug, and the level of one or more of T2DMARKER proteins, nucleic acids, polymorphisms, metabolites or other analytes can be determined. The level of one or more T2DMARKERS can be compared to sample derived from the subject before and after treatment or exposure to a therapeutic agent or a drug, or can be compared to samples derived from one or more subjects who have shown improvements in Diabetes or pre-Diabetes risk factors (e.g., clinical parameters or traditional laboratory risk factors) as a result of such treatment or exposure.

Agents for reducing the risk of Diabetes, pre-Diabetes, pre-diabetic conditions, or diabetic complications include, without limitation of the following, insulin, hypoglycemic agents, anti-inflammatory agents, lipid reducing agents, anti-hypertensives such as calcium channel blockers, beta-adrenergic receptor blockers, cyclooxygenase-2 inhibitors, angiotensin system inhibitors, ACE inhibitors, rennin inhibitors, together with other common risk factor modifying agents (herein “Diabetes-modulating drugs”).

“Insulin” includes rapid acting forms, such as Insulin lispro rDNA origin: HUMALOG (1.5 mL, 10 mL, Eli Lilly and Company, Indianapolis, Ind.), Insulin Injection (Regular Insulin) form beef and pork (regular ILETIN I, Eli Lilly], human: rDNA: HUMULIN R (Eli Lilly), NOVOLIN R (Novo Nordisk, New York, N.Y.), Semisynthetic: VELOSULIN Human (Novo Nordisk), rDNA Human, Buffered: VELOSULIN BR, pork: regular Insulin (Novo Nordisk), purified pork: Pork Regular ILETIN II (Eli Lilly), Regular Purified Pork Insulin (Novo Nordisk), and Regular (Concentrated) ILETIN II U-500 (500 units/mL, Eli Lilly); intermediate-acting forms such as Insulin Zinc Suspension, beef and pork: LENTE ILETIN G I (Eli Lilly), Human, rDNA: HUMULIN L (Eli Lilly), NOVOLIN L (Novo Nordisk), purified pork: LENTE ILETIN II (Eli Lilly), Isophane Insulin Suspension (NPH): beef and pork: NPH ILETIN I (Eli Lilly), Human, rDNA: HUMULIN N (Eli Lilly), Novolin N (Novo Nordisk), purified pork: Pork NPH Iletin II (Eli Lilly), NPH-N (Novo Nordisk); and long-acting forms such as Insulin zinc suspension, extended (ULTRALENTE, Eli Lilly), human, rDNA: HUMULIN U (Eli Lilly).

“Hypoglycemic” agents are preferably oral hypoglycemic agents and include, without limitation, first-generation sulfonylureas: Acetohexarnide (Dymelor), Chlorpropamide (Diabinese), Tolbutamide (Orinase); second-generation sulfonylureas: Glipizide (Glucotrol, Glucotrol XL), Glyburide (Diabeta; Micronase; Glynase), Glimepiride (Amaryl); Biguanides: Metformin (Glucophage); Alpha-glucosidase inhibitors: Acarbose (Precose), Miglitol (Glyset), Thiazolidinediones: Rosiglitazone (Avandia), Pioglitazone (Actos), Troglitazone (Rezulin); Meglitinides: Repaglinide (Prandin); and other hypoglycemics such as Acarbose; Buformin; Butoxamine Hydrochloride; Camiglibose; Ciglitazone; Englitazone Sodium; Darglitazone Sodium; Etoformin Hydrochloride; Gliamilide; Glibomuride; Glicetanile Gliclazide Sodium; Gliflumide; Glucagon; Glyhexamide; Glymidine Sodium; Glyoctamide; Glyparamide; Linogliride; Linogliride Fumarate; Methyl Palmoxirate; Palmoxirate Sodium; Pirogliride Tartrate; Proinsulin Human;; Seglitide Acetate; Tolazamide; Tolpyrramide; Zopolrestat.

“Anti-inflammatory” agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium. An important anti-inflammatory agent is aspirin.

Preferred anti-inflammatory agents are cytokine inhibitors. Important cytokine inhibitors include cytokine antagonists (e.g., IL-6 receptor antagonists), aza-alkyl lysophospholipids (AALP), and Tumor Necrosis Factor-alpha (TNF-alpha) inhibitors, such as anti-TNF-alpha antibodies, soluble TNF receptor, TNF-alpha, anti-sense nucleic acid molecules, multivalent guanylhydrazone (CNI-1493), N-acetylcysteine, pentoxiphylline, oxpentifylline, carbocyclic nucleoside analogues, small molecule S9a, RP 55778 (a TNF-alpha synthesis inhibitor), Dexanabinol (HU-211, is a synthetic cannabinoid devoid of cannabimimetic effects, inhibits TNF-alpha production at a post-transcriptional stage), MDL 201,449A (9-[(1R, 3R)-trans-cyclopentan-3-ol]adenine, and trichodimerol (BMS-182123). Preferred TNF-alpha inhibitors are Etanercept (ENBREL, Immunex, Seattle) and Infliximab (REMICADE, Centocor, Malvern, Pa.).

“Lipid reducing agents” include gemfibrozil, cholystyramine, colestipol, nicotinic acid, and HMG-CoA reductase inhibitors. HMG-CoA reductase inhibitors useful for administration, or co-administration with other agents according to the invention include, but are not limited to, simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402, the disclosures of which patents are incorporated herein by reference.

“Calcium channel blockers” are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11 -2933), phenytoin, barbiturates, and the peptides dynorphin, omega-conotoxin, and omega-agatoxin, and the like and/or pharmaceutically acceptable salts thereof.

“Beta-adrenergic receptor blocking agents” are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hedroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hyd-roxypropoxy)-3-pyridenecarbonitrilHCl, 1-butylamino-3-(2,5-dichlorophenoxy-)-2-propanol, 1-isopropylamino-3-(4-(2-cyclopropylmethoxyethyl)phenoxy)-2-propanol, 3-isopropylarnino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

A number of selective “COX-2 inhibitors” are known in the art and include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 “N-benzyl indol-3-yl butanoic acid derivatives as cyclooxygenase inhibitors”; U.S. Pat. No. 5,677,318 “Diphenyl-1,2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-dihy-drofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenyl-heterocycles as cyclooxygenase-2 and 5-lipoxygenase inhibitors.”

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

“Angiotensin II antagonists” are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include: peptidic compounds (e.g., saralasin, [(San¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo [4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alany-1-(4, thiazolyl)-L-alanyl (35,45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl)methyl] 1H-imidazole-5-yl[methylan-e]-2-thiophenepropanoic acid, Smith Kline Beecham Pharmaceuticals, Pa.); Losartan (DUP753/MK954, DuPont Merck Pharmaceutical Company); Remikirin (RO42-5892, F. Hoffman LaRoche AG); A.sub.2 agonists (Marion Merrill Dow) and certain non-peptide heterocycles (G. D. Searle and Company).

“Angiotensin converting enzyme (ACE) inhibitors” include amino acids and derivatives thereof, peptides, including di- and tri-peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

“Renin inhibitors” are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di- and tri-peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

Other diabetes-modulating drugs include, but are not limited to, lipase inhibitors such as cetilistat (ATL-962); synthetic amylin analogs such as Symlin pramlintide with or without recombinant leptin; sodium-glucose cotransporter 2 (SGLT2) inhibitors like sergliflozin (869682; KGT-1251), YM543, dapagliflozin, GlaxoSmithKline molecule 189075, and Sanofi-Aventis molecule AVE2268; dual adipose triglyceride lipase and P13 kinase activators like Adyvia (ID 1101); antagonists of neuropeptide Y2, Y4, and Y5 receptors like Nastech molecule PYY3-36, synthetic analog of human hormones PYY3-36 and pancreatic polypeptide (7TM molecule TM30338); Shionogi molecule S-2367; cannabinoid CB1 receptor antagonists such as rimonabant (Acomplia), taranabant, CP-945,598, Solvay molecule SLV319, Vemalis molecule V24343; hormones like oleoyl-estrone; inhibitors of serotonin, dopamine, and norepinephrine (also known in the art as “triple monoamine reuptake inhibitors”) like tesofensine (Neurosearch molecule NS2330); inhibitors of norepinephrine and dopamine reuptake, like Contrave (bupropion plus opioid antagonist naltrexone) and Excalia (bupropion plus anticonvulsant zonisaminde); inhibitors of 11β-hydroxysteroid dehydrogenase type 1 (11b-HSD1) like Incyte molecule INCB13739; inhibitors of cortisol synthesis such as ketoconazole (DiObex molecule DIO-902); inhibitors of gluconeogenesis such as Metabasis/Daiichi molecule CS-917; glucokinase activators like Roche molecule R1440; antisense inhibitors of protein tyrosine phosphatase-1B such as ISIS 113715; as well as other agents like NicOx molecule NCX 4016; injections of gastrin and epidermal growth factor (EGF) analogs such as Islet Neogenesis Therapy (E1-I.N.T.); and betahistine (Obecure molecule OBE101).

A subject cell (i.e., a cell isolated from a subject) can be incubated in the presence of a candidate agent and the pattern of T2DMARKER expression in the test sample is measured and compared to a reference profile, e.g., a Diabetes reference expression profile or a non-Diabetes reference expression profile or an index value or baseline value. The test agent can be any compound or composition or combination thereof. For example, the test agents are agents frequently used in Diabetes treatment regimens and are described herein.

Additionally, any of the aforementioned methods can be used separately or in combination to assess if a subject has shown an “improvement in Diabetes risk factors” or moved within the risk spectrum of pre-Diabetes. Such improvements include, without limitation, a reduction in body mass index (BMI), a reduction in blood glucose levels, an increase in HDL levels, a reduction in systolic and/or diastolic blood pressure, an increase in insulin levels, or combinations thereof.

A subject suffering from or at risk of developing Diabetes or a pre-diabetic condition may also be suffering from or at risk of developing arteriovascular disease, hypertension, or obesity. Type 2 Diabetes in particular and arteriovascular disease have many risk factors in common, and many of these risk factors are highly correlated with one another. The relationship s among these risk factors may be attributable to a small number of physiological phenomena, perhaps even a single phenomenon. Subjects suffering from or at risk of developing Diabetes, arteriovascular disease, hypertension or obesity are identified by methods known in the art.

Because of the interrelationship between Diabetes and arteriovascular disease, some or all of the individual T2DMARKERS and T2DMARKER panels of the present invention may overlap or be encompassed by biomarkers of arteriovascular disease, and indeed may be useful in the diagnosis of the risk of arteriovascular disease.

Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having Diabetes, pre-Diabetes, or a pre-diabetic condition, or at risk for Diabetes, pre-Diabetes, or a pre-diabetic condition, is based on whether the subjects have an “effective amount” or a “significant alteration” in the levels of a T2DMARKER. By “effective amount” or “significant alteration,” it is meant that the measurement of the T2DMARKER is different than the predetermined cut-off point (or threshold value) for that T2DMARKER and therefore indicates that the subject has Diabetes, pre-Diabetes, or a pre-diabetic condition for which the T2DMARKER is a determinant. The difference in the level of T2DMARKER between normal and abnormal is preferably statistically significant. As noted below, and without any limitation of the invention, achieving statistical significance, and thus the preferred analytical and clinical accuracy, generally but not always requires that combinations of several T2DMARKERS be used together in panels and combined with mathematical algorithms in order to achieve a statistically significant T2DMARKER index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining the clinically significant presence of T2DMARKERS, which thereby indicates the presence of Diabetes, pre-Diabetes, or a pre-diabetic condition) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy” , it is meant a test or assay in which the AUC (area under the ROC curve for the test or assay) is at least 0.80, desirably at least 0.85, more desirably at least 0.875, preferably at least 0.90, more preferably at least 0.925, and most preferably at least 0.95.

The predictive value of any test depends both on the sensitivity and specificity of the test, and on the prevalence of the condition in the population being tested. This notion, based on Bayes' theorem, provides that the greater the likelihood that the condition being screened for is present in a subject or in the population (pre-test probability), the greater the validity of a positive test and the greater the likelihood that the result is a true positive. Thus, the problem with using any test in any population where there is a low likelihood of the condition being present is that a positive result has more limited value (i.e., a positive test is more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing Diabetes, pre-Diabetes, or a pre-diabetic condition and the bottom quartile comprising the group of subjects having the lowest relative risk for developing Diabetes, pre-Diabetes, or a pre-diabetic condition. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future cardiovascular events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as pre-Diabetes) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the T2DMARKERS of the invention allows for one of skill in the art to use the T2DMARKERS to diagnose or identify subjects with a predetermined level of predictability and performance.

Relative Performance of the Invention

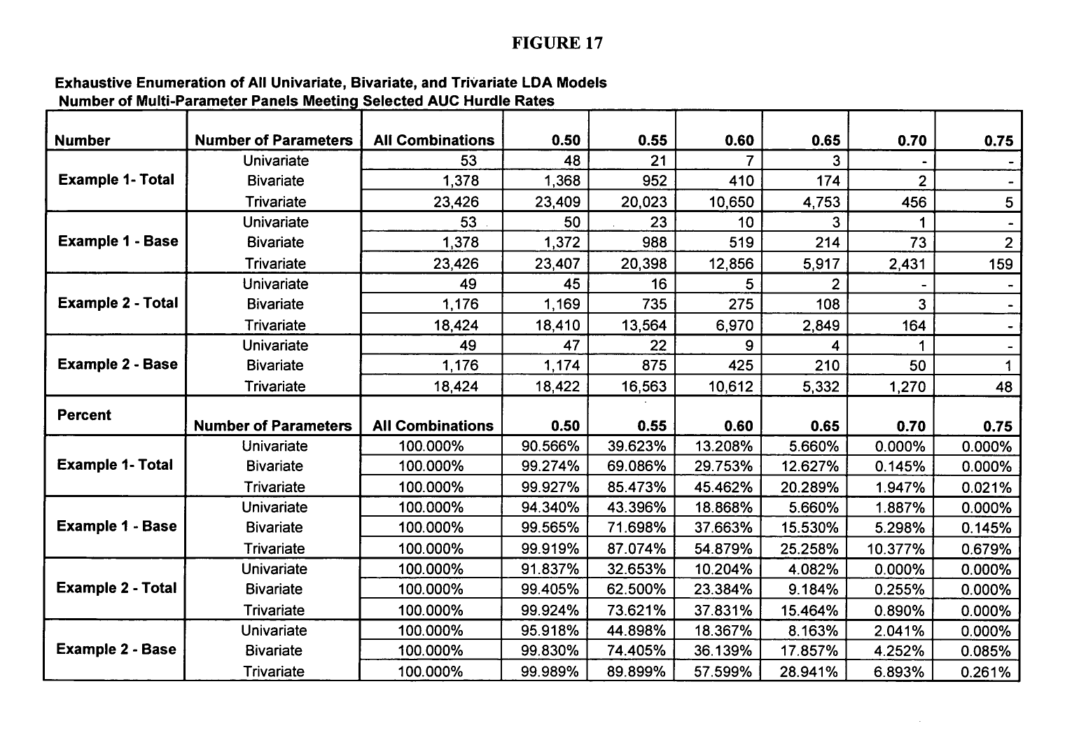

Only a minority of individual T2DMARKERS achieve an acceptable degree of diagnostic accuracy as defined above. Using a representative list of T2DMARKERS in each study, an exhaustive analysis of all potential univariate, bivariate, and trivariate combinations was used to derive a best fit LDA model to predict risk of conversion to Diabetes in each of the Example populations (see FIG. 17). For every possible T2DMARKER combination of a given panel size an LDA model was developed and then analyzed for its AUC statistics.

It is immediately apparent from the figure that there is a very low likelihood of high accuracy individual biomarkers, and even high accuracy combinations utilizing multiple biomarkers are infrequent. As demonstrated in FIG. 17, none of the individual T2DMARKERS, out of the 53 and 49 T2DMARKERS tested in Example 1 and Example 2, respectively, presented herein, achieved an AUC of 0.75 for the prediction of Diabetesin a best fit univariate model. The individual T2DMARKER parameters tested included many of the traditional laboratory risk factors and clinical parameters commonly used in global risk assessment and indices for Diabetes and arteriovascular disease.

Only two single T2DMARKERS, fasting glucose and insulin, even achieved an AUC of 0.70 in a univariate model; neither of these two biomarkers consistently did so in all of the population cohorts in the presented studies. Despite this lack of a very high level of diagnostic accuracy, fasting glucose remains the most common method of predicting the risk of Diabetes, and furthermore remains the primary method and definition used for the diagnosis of frank Diabetes.

In the Examples, achieving an accuracy defined by an AUC of 0.75 or above required a minimum combination of two or more biomarkers as taught in the invention herein. Across all of the examples, only three such two T2DMARKER combinations yielded bivariate models which met this hurdle, and only when used within the Base population cohorts of each Example, which had more selected (narrower) population selection (including only those with both a BMI greater than or equal to 25 and age greater than or equal to 39) than the total population of each Example. Such two biomarker combinations occurred at an approximate rate of only one in a thousand potential combinations.

However, as demonstrated above, several of the other biomarkers are useful in trivariate combinations of three T2DMARKERS, many of which achieved both acceptable performance either with or without including either glucose or insulin. Notably, in two separate studies, a representative set of 53 and 49 biomarkers selected out of the 266 T2DMARKERS, clinical parameters and traditional laboratory risk factors, were tested, and of these, certain combinations of three or more T2DMARKERS were found to exhibit superior performance. These are key aspects of the invention.

Notably, this analysis of FIG. 17 demonstrated that no single biomarker was required to practice the invention at an acceptable level of diagnostic accuracy, although several individually identified biomarkers are parts of the most preferred embodiments as disclosed below. It is a feature of the invention that the information lost due to removing one T2DMARKER can often be replaced through substitution with one or more other T2DMARKERS, and generically by increasing the panel size, subject to the need to increase the study size in order for studies examining very large models encompassing many T2DMARKERS to remain statistically significant. It is also a feature of the invention that overall performance and accuracy can often be improved by adding additional biomarkers (e.g., T2DMARKERS, traditional laboratory risk factors, and clinical parameters) as additional inputs to a formula or model, as demonstrated above in the relative performance of univariate, bivariate, and trivariate models, and below in the performance of larger models.

The ultimate determinant and gold standard of true risk of conversion to Diabetes is actual conversions within a sufficiently large study population and observed over the length of time claimed, as was done in the Examples contained herein. However, this is problematic, as it is necessarily a retrospective point of view. As a result, subjects suffering from or at risk of developing Diabetes, pre-Diabetes, or a pre-diabetic condition are commonly diagnosed or identified by methods known in the art, generally using either traditional laboratory risk factors or other non-analyte clinical parameters, and future risk is estimated based on historical experience and registry studies. Such methods include, but are not limited to, measurement of systolic and diastolic blood pressure, measurements of body mass index, in vitro determination of total cholesterol, LDL, HDL, insulin, and glucose levels from blood samples, oral glucose tolerance tests, stress tests, measurement of high sensitivity C-reactive protein (CRP), electrocardiogram (ECG), c-peptide levels, anti-insulin antibodies, anti-beta cell-antibodies, and glycosylated hemoglobin (HBA1c).

For example, Diabetes is frequently diagnosed by measuring fasting blood glucose, insulin, or HBA1c levels. Normal adult glucose levels are 60-126 mg/dl. Normal insulin levels are 7 mU/mL±3 mU. Normal HBA1c levels are generally less than 6%. Hypertension is diagnosed by a blood pressure consistently at or above 140/90. Risk of arteriovascular disease can also be diagnosed by measuring cholesterol levels. For example, LDL cholesterol above 137 or total cholesterol above 200 is indicative of a heightened risk of arteriovascular disease. Obesity is diagnosed for example, by body mass index. Body mass index (BMI) is measured (kg/m² (or lb/in²×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e. fat mass impedes current), estimates % fat) is measured. The parameters for normal, overweight, or obese individuals is as follows: Underweight: BMI <18.5; Normal: BMI 18.5 to 24.9; Overweight: BMI=25 to 29.9. Overweight individuals are characterized as having a waist circumference of >94 cm for men or >80 cm for women and waist to hip ratios of ≧0.95 in men and ≧0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of ≧35.

As noted above, risk prediction for Diabetes, pre-Diabetes, or a pre-diabetic condition can also encompass risk prediction algorithms and computed indices that assess and estimate a subject's absolute risk for developing Diabetes, pre-Diabetes, or a pre-diabetic diabetic condition with reference to a historical cohort. Risk assessment using such predictive mathematical algorithms and computed indices has increasingly been incorporated into guidelines for diagnostic testing and treatment, and encompass indices obtained from and validated with, inter alia, multi-stage, stratified samples from a representative population.

Despite the numerous studies and algorithms that have been used to assess the risk of Diabetes, pre-Diabetes, or a pre-diabetic condition, the evidence-based, multiple risk factor assessment approach is only moderately accurate for the prediction of short- and long-term risk of manifesting Diabetes, pre-Diabetes, or a pre-diabetic condition in individual asymptomatic or otherwise healthy subjects. Such risk prediction algorithms can be advantageously used in combination with the T2DMARKERS of the present invention to distinguish between subjects in a population of interest to determine the risk stratification of developing Diabetes, pre-Diabetes, or a pre-diabetic condition. The T2DMARKERS and methods of use disclosed herein provide tools that can be used in combination with such risk prediction algorithms to assess, identify, or diagnose subjects who are asymptomatic and do not exhibit the conventional risk factors.

The data derived from risk factors, risk prediction algorithms and from the methods of the present invention can be combined and compared by known statistical techniques in order to compare the relative performance of the invention to the other techniques.

Furthermore, the application of such techniques to panels of multiple T2DMARKERS is encompassed by or within the ambit of the present invention, as is the use of such combinations and formulae to create single numerical “risk indices” or “risk scores” encompassing information from multiple T2DMARKER inputs.

Risk Markers of the Invention (T2DMARKERS)

The biomarkers and methods of the present invention allow one of skill in the art to identify, diagnose, or otherwise assess those subjects who do not exhibit any symptoms of Diabetes, pre-Diabetes, or a pre-diabetic condition, but who nonetheless may be at risk for developing Diabetes, pre-Diabetes, or experiencing symptoms characteristic of a pre-diabetic condition.

Two hundred and sixty-six (266) analyte-based biomarkers have been identified as being found to have altered or modified presence or concentration levels in subjects who have Diabetes, or who exhibit symptoms characteristic of a pre-diabetic condition, or have pre-Diabetes (as defined herein), including such subjects as are insulin resistant, have altered beta cell function or are at risk of developing Diabetes based upon known clinical parameters or traditional laboratory risk factors, such as family history of Diabetes, low activity level, poor diet, excess body weight (especially around the waist), age greater than 45 years, high blood pressure, high levels of triglycerides, HDL cholesterol of less than 35, previously identified impaired glucose tolerance, previous Diabetes during pregnancy (Gestational Diabetes Mellitus or GDM) or giving birth to a baby weighing more than nine pounds, and ethnicity.

Table 1 comprises the two-hundred and sixty-six (266) T2DMARKERS, which are analyte-based biomarkers of the present invention. One skilled in the art will recognize that the T2DMARKERS presented herein encompasses all forms and variants, including but not limited to, polymorphisms, isoforms, mutants, derivatives, precursors including nucleic acids and pro-proteins, cleavage products, receptors (including soluble and transmembrane receptors), ligands, protein-ligand complexes, and post-translationally modified variants (such as cross-linking or glycosylation), fragments, and degradation products, as well as any multi-unit nucleic acid, protein, and glycoprotein structures comprised of any of the T2DMARKERS as constituent subunits of the fully assembled structure.

TABLE 1
T2DMARKERS

| | | | Entrez Gene |
|---|---|---|---|
| T2DMARKER | Official Name | Common Name | Link |
| 1 | ATP-binding cassette, sub-family C | sulfonylurea receptor (SUR1), | ABCC8 |
| | (CFTR/MRP), member 8 | HI; SUR; HHF1; MRP8; | |
| | | PHHI; SUR1; ABC36; HRINS | |
| 2 | ATP-binding cassette, sub-family C | sulfonylurea receptor (SUR2a), | ABCC9 |
| | (CFTR/MRP), member 9 | SUR2; ABC37; CMD1O; | |
| | | FLJ36852 | |
| 3 | angiotensin I converting enzyme | angiotensin-converting enzyme | ACE |
| | (peptidyl-dipeptidase A) 1 | (ACE) - ACE1, CD143, DCP, | |
| | | DCP1, CD143 antigen; | |
| | | angiotensin I converting | |
| | | enzyme; angiotensin | |
| | | converting enzyme, somatic | |
| | | isoform; carboxycathepsin; | |
| | | dipeptidyl carboxypeptidase 1; | |
| | | kininase II; peptidase P; | |
| | | peptidyl-dipeptidase A; | |
| | | testicular ECA | |
| 4 | adenylate cyclase activating | adenylate cyclase activating | ADCYAP1 |
| | polypeptide 1 (pituitary) | polypeptide | |
| 5 | adiponectin, C1Q and collagen | Adiponectin - ACDC, | ADIPOQ |
| | domain containing | ACRP30, APM-1, APM1, | |
| | | GBP28, glycosylated | |
| | | adiponectin, adiponectin, | |
| | | adipocyte, C1Q and collagen | |
| | | domain containing; adipocyte, | |
| | | C1Q and collagen domain- | |
| | | containing; adiponectin; | |
| | | adipose most abundant gene | |
| | | transcript 1; gelatin-binding | |
| | | protein 28 | |
| 6 | adiponectin receptor 1 | G Protein Coupled Receptor | ADIPOR1 |
| | | AdipoR1 - ACDCR1, CGI-45, | |
| | | PAQR1, TESBP1A | |
| 7 | adiponectin receptor 2 | G Protein Coupled Receptor | ADIPOR2 |
| | | AdipoR2 - ACDCR2, PAQR2 | |
| 8 | adrenomedullin | adrenomedullin - AM, | ADM |
| | | preproadrenomedullin | |
| 9 | adrenergic, beta-2-, receptor, surface | G Protein-Coupled Beta-2 | ADRB2 |
| | | Adrenoceptor - ADRB2R, | |
| | | ADRBR, B2AR, BAR, | |
| | | BETA2AR, beta-2 adrenergic | |
| | | receptor; beta-2 adrenoceptor; | |
| | | catecholamine receptor | |
| 10 | advanced glycosylation end product- | RAGE - advanced | AGER |
| | specific receptor | glycosylation end product- | |
| | | specific receptor RAGE3; | |
| | | advanced glycosylation end | |
| | | product-specific receptor | |
| | | variant sRAGE1; advanced | |
| | | glycosylation end product- | |
| | | specific receptor variant | |
| | | sRAGE2; receptor for | |
| | | advanced glycosylation end- | |
| | | products; soluble receptor | |
| 11 | agouti related protein homolog | AGRT, ART, ASIP2, & | AGRP |
| | (mouse) | Agouti-related transcript, | |
| | | mouse, homolog of; agouti | |
| | | (mouse) related protein; agouti | |
| | | related protein homolog | |
| 12 | angiotensinogen (serpin peptidase | angiotensin I; pre- | AGT |
| | inhibitor, clade A, member 8) | angiotensinogen; angiotensin II | |
| | | precursor; angiotensinogen | |
| | | (serine (or cysteine) peptidase | |
| | | inhibitor, clade A, member 8); | |
| | | angiotensinogen (serine (or | |
| | | cysteine) proteinase inhibitor, | |
| | | clade A (alpha-1 | |
| | | antiproteinase, antitrypsin), | |
| | | member 8) | |
| 13 | angiotensin II receptor, type 1 | G protein-Coupled Receptor | AGTR1 |
| | | AGTR1A - AG2S, AGTR1A, | |
| | | AGTR1B, AT1, AT1B, | |
| | | AT2R1, AT2R1A, AT2R1B, | |
| | | HAT1R, angiotensin receptor | |
| | | 1; angiotensin receptor 1B; | |
| | | type-1B angiotensin II receptor | |
| 14 | angiotensin II receptor-associated | angiotensin II - ATRAP, ATI | AGTRAP |
| | protein | receptor-associated protein; | |
| | | angiotensin II, type I receptor- | |
| | | associated protein | |
| 15 | alpha-2-HS-glycoprotein | A2HS, AHS, FETUA, HSGA, | AHSG |
| | | Alpha-2HS-glycoprotein; | |
| | | fetuin-A | |
| 16 | v-akt murine thymoma viral | Ser/Thr kinase Akt - PKB, | AKT1 |
| | oncogene homolog 1 | PRKBA, RAG, RAG-ALPHA, | |
| | | RAG-alpha serine/threonine- | |
| | | protein kinase; murine | |
| | | thymoma viral (v-akt) | |
| | | oncogene homolog-1; protein | |
| | | kinase B; rac protein kinase | |
| | | alpha | |
| 17 | v-akt murine thymoma viral | PKBBETA, PRKBB, RAC- | AKT2 |
| | oncogene homolog 2 | BETA, Murine thymoma viral | |
| | | (v-akt) homolog-2; rac protein | |
| | | kinase beta | |
| 18 | albumin | Ischemia-modified albumin | ALB |
| | | (IMA) - cell growth inhibiting | |
| | | protein 42; growth-inhibiting | |
| | | protein 20; serum albumin | |
| 19 | Alstrom syndrome 1 | ALSS | ALMS1 |
| 20 | archidonate 12-lipoxygenase | LOG12, 12(S)-lipoxygenase; | ALOX12 |
| | | platelet-type 12- | |
| | | lipoxygenase/arachidonate 12- | |
| | | lipoxygenase | |
| 21 | Angiogenin, ribonuclease, RNase A | Angiogenin, MGG71966, | ANG |
| | family, 5 | RNASE4, RNASE5, | |
| | | angiogenin, ribonuclease, | |
| | | RNase A family, 5 | |
| 22 | ankyrin repeat domain 23 | DARP, MARP3, Diabetes | ANKRD23 |
| | | related ankyrin repeat protein; | |
| | | muscle ankyrin repeat protein 3 | |
| 23 | apelin, AGTRL 1 Ligand | XNPEP2, apelin, peptide | APLN |
| | | ligand for APJ receptor | |
| 24 | apolipoprotein A-I | apolipoproteins A-1 and B, | APOA1 |
| | | amyloidosis; apolipoprotein A- | |
| | | I, preproprotein; | |
| | | apolipoprotein A1; | |
| | | preproapolipoprotein | |
| 25 | apolipoprotein A-II | Apolipoprotein A-II | APOA2 |
| 26 | apolipoprotein B (including Ag(x) | apolipoproteins A-1 and B - | APOB |
| | antigen) | Apolipoprotein B, FLDB, | |
| | | apoB-100; apoB-48; | |
| | | apolipoprotein B; | |
| | | apolipoprotein B48 | |
| 27 | apolipoprotein E | APO E - AD2, apoprotein, | APOE |
| | | Alzheimer disease 2 | |
| | | (APOE*E4-associated, late | |
| | | onset); apolipoprotein E | |
| | | precursor; apolipoprotein E3 | |
| 28 | aryl hydrocarbon receptor nuclear | dioxin receptor, nuclear | ARNT |
| | translocator | translocator; hypoxia-inducible | |
| | | factor 1, beta subunit | |
| 29 | Aryl hydrocarbon receptor nuclear | Bmall, TIC; JAP3; MOP3; | ARNTL |
| | translocator-like | BMAL1; PASD3; BMAL1c; | |
| | | bHLH-PAS protein JAP3; | |
| | | member of PAS superfamily 3; | |
| | | ARNT-like protein 1, brain | |
| | | and muscle; basic-helix-loop- | |
| | | helix-PAS orphan MOP3 | |
| 30 | arrestin, beta 1 | beta arrestin - ARB1, ARR1, | ARRB1 |
| | | arrestin beta 1 | |
| 31 | arginine vasopressin (neurophysin II, | copeptin - ADH, ARVP, AVP- | AVP |
| | antidiuretic hormone, Diabetes | NPII, AVRP, VP, arginine | |
| | insipidus, neurohypophyseal) | vasopressin-neurophysin II; | |
| | | vasopressin-neurophysin II- | |
| | | copeptin, vasopressin | |
| 32 | bombesin receptor subtype 3 | G-protein coupled receptor; | BRS3 |
| | | bombesin receptor subtype 3 | |
| 33 | betacellulin | betacellulin | BTC |
| 34 | benzodiazepine receptor (peripheral) | PBR - DBI, IBP, MBR, PBR, | BZRP |
| | | PKBS, PTBR, mDRC, pk18, | |
| | | benzodiazepine peripheral | |
| | | binding site; mitochondrial | |
| | | benzodiazepine receptor; | |
| | | peripheral benzodiazapine | |
| | | receptor; peripheral | |
| | | benzodiazepine receptor; | |
| | | peripheral-type benzodiazepine | |
| | | receptor | |
| 35 | complement component 3 | complement C3 - acylation- | C3 |
| | | stimulating protein cleavage | |
| | | product; complement | |
| | | component C3, ASP; | |
| | | CPAMD1 | |
| 36 | complement component 4A | complement C4 - C4A | C4A |
| | (Rodgers blood group) | anaphylatoxin; Rodgers form | |
| | | of C4; acidic C4; c4 | |
| | | propeptide; complement | |
| | | component 4A; complement | |
| | | component C4B | |
| 37 | complement component 4B (Childo | C4A, C4A13, C4A91, C4B1, | C4B |
| | blood group) | C4B12, C4B2, C4B3, C4B5, | |
| | | C4F, CH, CO4, CPAMD3, C4 | |
| | | complement C4d region; | |
| | | Chido form of C4; basic C4; | |
| | | complement C4B; complement | |
| | | component 4B; complement | |
| | | component 4B, centromeric; | |
| | | complement component 4B, | |
| | | telomeric; complement | |
| | | component C4B | |
| 38 | complement component 5 | anaphylatoxin C5a analog - | C5 |
| | | CPAMD4 | |
| 39 | Calpain-10 | calcium-activated neutral | CAPN10 |
| | | protease | |
| 40 | cholecystokinin | cholecystokinin | CCK |
| 41 | cholecystokinin (CCK)-A receptor | CCK-A; CCK-A; CCKRA; | CCKAR |
| | | CCK1-R; cholecystokinin-1 | |
| | | receptor; cholecystokinin | |
| | | type-A receptor | |
| 42 | chemokine (C—C motif) ligand 2 | Monocyte chemoattractant | CCL2 |
| | | protein-1 (MCP-1) - GDCF-2, | |
| | | GDCF-2 HC11, HC11, | |
| | | HSMCR30, MCAF, MCP-1, | |
| | | MCP1, SCYA2, SMC-CF, | |
| | | monocyte chemoattractant | |
| | | protein-1; monocyte | |
| | | chemotactic and activating | |
| | | factor; monocyte chemotactic | |
| | | protein 1, homologous to | |
| | | mouse Sig-je; monocyte | |
| | | secretory protein JE; small | |
| | | inducible cytokine A2; small | |
| | | inducible cytokine A2 | |
| | | (monocyte chemotactic protein | |
| | | 1, homologous to mouse Sig- | |
| | | je); small inducible cytokine | |
| | | subfamily A (Cys-Cys), | |
| | | member 2 | |
| 43 | CD14 molecule | CD14 antigen - monocyte | CD14 |
| | | receptor | |
| 44 | CD163 molecule | CD163-M130, MM130- | CD163 |
| | | CD163 antigen; macrophage- | |
| | | associated antigen, | |
| | | macrophage-specific antigen | |
| 45 | CD36 molecule (thrombospondin | fatty acid translocase, FAT; | CD36 |
| | receptor) | GP4; GP3B; GPIV; PASIV; | |
| | | SCARB3, PAS-4 protein; | |
| | | collagen type I; glycoprotein | |
| | | IIIb; cluster determinant 36; | |
| | | fatty acid translocase; | |
| | | thrombospondin receptor; | |
| | | collagen type I receptor; | |
| | | platelet glycoprotein IV; | |
| | | platelet collagen receptor; | |
| | | scavenger receptor class B, | |
| | | member 3; leukocyte | |
| | | differentiation antigen CD36; | |
| | | CD36 antigen (collagen type I | |
| | | receptor, thrombospondin | |
| | | receptor) | |
| 46 | CD38 molecule | T10; CD38 antigen (p45); | CD38 |
| | | cyclic ADP-ribose hydrolase; | |
| | | ADP-ribosyl cyclase/cyclic | |
| | | ADP-ribose hydrolase | |
| 47 | CD3d molecule, delta (CD3-TCR | CD3-DELTA, T3D, CD3D | CD3D |
| | complex) | antigen, delta polypeptide; | |
| | | CD3d antigen, delta | |
| | | polypeptide (TiT3 complex); | |
| | | T-cell receptor T3 delta chain | |
| 48 | CD3g molecule, gamma (CD3-TCR | T3G; CD3-GAMMA, T3G, | CD3G |
| | complex) | CD3G gamma; CD3g antigen, | |
| | | gamma polypeptide (TiT3 | |
| | | complex); T-cell antigen | |
| | | receptor complex, gamma | |
| | | subunit of T3; T-cell receptor | |
| | | T3 gamma chain; T-cell | |
| | | surface glycoprotein CD3 | |
| | | gamma chain precursor | |
| 49 | CD40 molecule, TNF receptor | Bp50, CDW40, TNFRSF5, | CD40 |
| | superfamily member 5 | p50, B cell surface antigen | |
| | | CD40; B cell-associated | |
| | | molecule; CD40 antigen; | |
| | | CD40 antigen (TNF receptor | |
| | | superfamily member 5); CD40 | |
| | | type II isoform; CD40L | |
| | | receptor; nerve growth factor | |
| | | receptor-related B-lymphocyte | |
| | | activation molecule; tumor | |
| | | necrosis factor receptor | |
| | | superfamily, member 5 | |
| 50 | CD40 ligand (TNF superfamily, | CD40 Ligand (CD40L) (also | CD40LG |
| | member 5, hyper-IgM syndrome) | called soluble CD40L vs. | |
| | | platelet-bound CD40L), | |
| | | CD154, CD40L, HIGM1, | |
| | | IGM, IMD3, T-BAM, | |
| | | TNFSF5, TRAP, gp39, | |
| | | hCD40L, CD40 antigen ligand; | |
| | | CD40 ligand; T-B cell- | |
| | | activating molecule; TNF- | |
| | | related activation protein; | |
| | | tumor necrosis factor (ligand) | |
| | | superfamily member 5; tumor | |
| | | necrosis factor (ligand) | |
| | | superfamily, member 5 (hyper- | |
| | | IgM syndrome); tumor | |
| | | necrosis factor ligand | |
| | | superfamily member 5 | |
| 51 | CD68 molecule | GP110; SCARD1; macrosialin; | CD68 |
| | | CD68 antigen; macrophage | |
| | | antigen CD68; scavenger | |
| | | receptor class D, member 1 | |
| 52 | cyclin-dependent kinase 5 | PSSALRE; cyclin-dependent | CDK5 |
| | | kinase 5 | |
| 53 | complement factor D (adipsin) | ADN, DF, PFD, C3 convertase | CFD |
| | | activator; D component of | |
| | | complement (adipsin); adipsin; | |
| | | complement factor D; | |
| | | properdin factor D | |
| 54 | CASP8 and FADD-like apoptosis | FLIP - caspase 8 inhibitor, | CFLAR |
| | regulator | CASH; FLIP; MRIT; CLARP; | |
| | | FLAME; Casper; c-FLIP; | |
| | | FLAME-1; I-FLICE; | |
| | | USURPIN; c-FLIPL; c-FLIPR; | |
| | | c-FLIPS; CASP8AP1, usurpin | |
| | | beta; FADD-like anti- | |
| | | apoptotic molecule; Inhibitor | |
| | | of FLICE; Caspase-related | |
| | | inducer of apoptosis; Caspase | |
| | | homolog; Caspase-like | |
| | | apoptosis regulatory protein | |
| 55 | Clock homolog (mouse) | clock protein; clock (mouse) | CLOCK |
| | | homolog; circadian locomoter | |
| | | output cycles kaput protein | |
| 56 | chymase 1, mast cell | chymase 1 - CYH, MCT1, | CMA1 |
| | | chymase 1 preproprotein | |
| | | transcript E; chymase 1 | |
| | | preproprotein transcript I; | |
| | | chymase, heart; chymase, mast | |
| | | cell; mast cell protease I | |
| 57 | cannabinoid receptor 1 (brain) | cannabinoid receptor 1 - | CNR1 |
| | | CANN6, CB-R, CB1, CB1A, | |
| | | CB1K5, CNR, central | |
| | | cannabinoid receptor | |
| 58 | cannabinoid receptor 2 (macrophage) | cannabmoid receptor 2 | CNR2 |
| | | (macrophage), CB2, CX5 | |
| 59 | cortistatin | CST-14; CST-17; CST-29; | CORT |
| | | cortistatin-14; cortistatin-17; | |
| | | cortistatin-29; preprocortistatin | |
| 60 | carnitine palmitoyltransferase I | CPT1; CPT1-L; L-CPT1, | CPT1A |
| | | carnitine palmitoyltransferase | |
| | | I; liver | |
| 61 | carnitine palmitoyltransferase II | CPT1, CPTASE | CPT2 |
| 62 | complement component (3b/4b) | complement receptor CR1; | CR1 |
| | receptor 1 | KN; C3BR; CD35; CD35 | |
| | | antigen; C3b/C4b receptor; | |
| | | C3-binding protein; Knops | |
| | | blood group antigen; | |
| | | complement component | |
| | | receptor 1; complement | |
| | | component (3b/4b) receptor 1, | |
| | | including Knops blood group | |
| | | system | |
| 63 | complement component (3d/Epstein | complement receptor CR2; | CR2 |
| | Barr virus) receptor 2 | C3DR; CD21 | |
| 64 | CREB binding protein (Rubinstein- | Cbp; CBP; RTS; RSTS, | CREBBP |
| | Taybi syndrome) | CREB-binding protein | |
| 65 | C-reactive protein, pentraxin-related | C-Reactive Protein, CRP, | CRP |
| | | PTX1 | |
| 66 | CREB regulated transcription | Torc2 (transcriptional | CRTC2 |
| | coactivator 2 | coactivator); transducer of | |
| | | regulated cAMP response | |
| | | element-binding protein | |
| | | (CREB) 2 | |
| 67 | colony stimulating factor 1 | M-CSF - colony stimulating | CSF1 |
| | (macrophage) | factor 1; macrophage colony | |
| | | stimulating factor | |
| 68 | cathepsin B | cathepsin B - procathepsin B, | CTSB |
| | | APPS; CPSB, APP secretase; | |
| | | amyloid precursor protein | |
| | | secretase; cathepsin B1; | |
| | | cysteine protease; | |
| | | preprocathepsin B | |
| 69 | cathepsin L | CATL, MEP, major excreted | CTSL |
| | | protein | |
| 70 | cytochrome P450, family 19, | ARO, ARO1, CPV1, CYAR, | CYP19A1 |
| | subfamily A, polypeptide 1 | CYP19, P-450AROM, | |
| | | aromatase; cytochrome P450, | |
| | | family 19; cytochrome P450, | |
| | | subfamily XIX (aromatization | |
| | | of androgens); estrogen | |
| | | synthetase; flavoprotein-linked | |
| | | monooxygenase; microsomal | |
| | | monooxygenase | |
| 71 | Dio-2, death inducer-obliterator 1 | death associated transcription | DIDO1 |
| | | factor 1; BYE1; DIO1; | |
| | | DATF1; DIDO2; DIDO3; | |
| | | DIO-1 | |
| 72 | dipeptidyl-peptidase 4 (CD26, | dipeptidylpeptidase IV - | DPP4 |
| | adenosine deaminase complexing | ADABP, ADCP2, CD26, | |
| | protein 2) | DPPIV, TP103, T-cell | |
| | | activation antigen CD26; | |
| | | adenosine deaminase | |
| | | complexing protein 2; | |
| | | dipeptidylpeptidase IV; | |
| | | dipeptidylpeptidase IV (CD26, | |
| | | adenosine deaminase | |
| | | complexing protein 2) | |
| 73 | epidermal growth factor (beta- | URG - urogastrone | EGF |
| | urogastrone) | | |
| 74 | early growth response 1 | zinc finger protein 225; | EGR1 |
| | | transcription factor ETR103; | |
| | | early growth response protein | |
| | | 1; nerve growth factor-induced | |
| | | protein A | |
| 75 | epididymal sperm binding protein 1 | E12, HE12, epididymal | ELSPBP1 |
| | | secretory protein | |
| 76 | ectonucleotide | ENPP1 - M6S1, NPP1, NPPS, | ENPP1 |
| | pyrophosphatase/phosphodiesterase 1 | PC-1, PCA1, PDNP1, Ly-41 | |
| | | antigen; alkaline | |
| | | phosphodiesterase 1; | |
| | | membrane component, | |
| | | chromosome 6, surface marker | |
| | | 1; phosphodiesterase | |
| | | I/nucleotide pyrophosphatase | |
| | | 1; plasma-cell membrane | |
| | | glycoprotein 1 | |
| 77 | E1A binding protein p300 | p300, E1A binding protein | EP300 |
| | | p300, E1A-binding protein, | |
| | | 300 kD; E1A-associated protein | |
| | | p300 | |
| 78 | coagulation factor XIII, A1 | Coagulation Factor XIII - | F13A1 |
| | polypeptide | Coagulation factor XIII A | |
| | | chain; Coagulation factor XIII, | |
| | | A polypeptide; TGase; | |
| | | (coagulation factor XIII, A1 | |
| | | polypeptide); coagulation | |
| | | factor XIII A1 subunit; factor | |
| | | XIIIa, coagulation factor XIII | |
| | | A1 subunit | |
| 79 | coagulation factor VIII, procoagulant | Factor VIII, AHF, F8 protein, | F8 |
| | component (hemophilia A) | F8B, F8C, FVIII, HEMA, | |
| | | coagulation factor VIII; | |
| | | coagulation factor VIII, | |
| | | isoform b; coagulation factor | |
| | | VIIIc; factor VIII F8B; | |
| | | procoagulant component, | |
| | | isoform b | |
| 80 | fatty acid binding protein 4, | fatty acid binding protein 4, | FABP4 |
| | adipocyte | adipocyte - A-FABP | |
| 81 | Fas (TNF receptor superfamily, | soluble Fas/APO-1 (sFas), | FAS |
| | member 6) | ALPS1A, APO-1, APT1, Apo- | |
| | | 1 Fas, CD95, FAS1, FASTM, | |
| | | TNFRSF6, APO-1 cell surface | |
| | | antigen; CD95 antigen; Fas | |
| | | antigen; apoptosis antigen 1; | |
| | | tumor necrosis factor receptor | |
| | | superfamily, member 6 | |
| 82 | Fas ligand (TNF superfamily, | Fas ligand (sFasL), APT1LG1, | FASLG |
| | member 6) | CD178, CD95L, FASL, | |
| | | TNFSF6, CD95 ligand; | |
| | | apoptosis (APO-1) antigen | |
| | | ligand 1; fas ligand; tumor | |
| | | necrosis factor (ligand) | |
| | | superfamily, member 6 | |
| 83 | free fatty acid receptor 1 | G protein-coupled receptor 40 - | FFAR1 |
| | | FFA1R, GPR40, G protein- | |
| | | coupled receptor 40 | |
| 84 | fibrinogen alpha chain | Fibrin, Fib2, fibrinogen, A | FGA |
| | | alpha polypeptide; fibrinogen, | |
| | | alpha chain, isoform alpha | |
| | | preproprotein; fibrinogen, | |
| | | alpha polypeptide | |
| 85 | forkhead box A2 | (Foxa2); HNF3B; TCF3B; | FOXA2 |
| | | hepatic nuclear factor-3-beta; | |
| | | hepatocyte nuclear factor 3, | |
| | | beta | |
| 86 | forkhead box O1A | FKH1; FKHR; FOXO1; | FOXO1A |
| | | forkhead (Drosophila) | |
| | | homolog 1 | |
| | | (rhabdomyosarcoma); | |
| | | forkhead, Drosophila, homolog | |
| | | of, in rhabdomyosarcoma | |
| 87 | ferritin | FTH; PLIF; FTHL6; PIG15; | FTH1 |
| | | apoferritin; placenta | |
| | | immunoregulatory factor; | |
| | | proliferation-inducing protein | |
| | | 15 | |
| 88 | glutamate decarboxylase 2 | glutamic acid decarboxylase | GAD2 |
| | | (GAD65) antibodies; | |
| | | Glutamate decarboxylase-2 | |
| | | (pancreas); glutamate | |
| | | decarboxylase 2 (pancreatic | |
| | | islets and brain, 65 kD) | |
| 89 | galanin | GALN; GLNN; galanin-related | GAL |
| | | peptide | |
| 90 | gastrin | gastrin - GAS | GAST |
| 91 | glucagon | glucagon-like peptide-1, GLP- | GCG |
| | | 1, GLP2, GRPP, glicentin- | |
| | | related polypeptide; glucagon- | |
| | | like peptide 1; glucagon-like | |
| | | peptide 2 | |
| 92 | glucokinase | hexokinase 4, maturity to onset | GCK |
| | | Diabetes of the young 2; GK; | |
| | | GLK; HK4; HHF3; HKIV; | |
| | | HXKP; MODY2 | |
| 93 | gamma-glutamyltransferase 1 | GGT; GTG; CD224; glutamyl | GGT1 |
| | | transpeptidase; gamma- | |
| | | glutamyl transpeptidase | |
| 94 | growth hormone 1 | growth hormone - GH, GH-N, | GH1 |
| | | GHN, hGH-N, pituitary | |
| | | growth hormone | |
| 95 | ghrelin/obestatin preprohormone | ghrelin - MTLRP, ghrelin, | GHRL |
| | | obestatin, ghrelin; ghrelin | |
| | | precursor; ghrelin, growth | |
| | | hormone secretagogue receptor | |
| | | ligand; motilin-related peptide | |
| 96 | gastric inhibitory polypeptide | glucose-dependent | GIP |
| | | insulinotropic peptide | |
| 97 | gastric inhibitory polypeptide | GIP Receptor | GIPR |
| | receptor | | |
| 98 | glucagon-like peptide 1 receptor | glucagon-like peptide 1 | GLP1R |
| | | receptor | |
| 99 | guanine nucleotide binding protein | G-protein beta-3 subunit - G | GNB3 |
| | (G protein), beta polypeptide 3 | protein, beta-3 subunit; GTP- | |
| | | binding regulatory protein | |
| | | beta-3 chain; guanine | |
| | | nucleotide-binding protein | |
| | | G(I)/G(S)/G(T) beta subunit 3; | |
| | | guanine nucleotide-binding | |
| | | protein, beta-3 subunit; | |
| | | hypertension associated | |
| | | protein; transducin beta chain 3 | |
| 100 | glutamic-pyruvate transaminase | glutamic-pyruvate | GPT |
| | (alanine aminotransferase) | transaminase (alanine | |
| | | aminotransferase), AAT1, | |
| | | ALT1, GPT1 | |
| 101 | gastrin releasing peptide (bombesin) | bombesin; BN; GRP-10; | GRP |
| | | proGRP; preproGRP; | |
| | | neuromedin C; pre-progastrin | |
| | | releasing peptide | |
| 102 | gelsolin (amyloidosis, Finnish type) | gelsolin | GSN |
| 103 | hemoglobin | CD31; alpha-1 globin; alpha-1- | HBA1 |
| | | globin; alpha-2 globin; alpha- | |
| | | 2-globin; alpha one globin; | |
| | | hemoglobin alpha 2; | |
| | | hemoglobin alpha-2; | |
| | | hemoglobin alpha-1 chain; | |
| | | hemoglobin alpha 1 globin | |
| | | chain, glycosylated | |
| | | hemoglobin, HBA1c | |
| 104 | hemoglobin, beta | HBD, beta globin | HBB |
| 105 | hypocretin (orexin) neuropeptide | orexin A; OX; PPOX | HCRT |
| | precursor | | |
| 106 | hepatocyte growth factor | Hepatocyte growth factor | HGF |
| | (hepapoietin A; scatter factor) | (HGF) - F-TCF, HGFB, | |
| | | HPTA, SF, fibroblast-derived | |
| | | tumor cytotoxic factor; | |
| | | hepatocyte growth factor; | |
| | | hepatopoietin A; lung | |
| | | fibroblast-derived mitogen; | |
| | | scatter factor | |
| 107 | hepatocyte nuclear factor 4, alpha | hepatocyte nuclear factor 4- | HNF4A |
| | | HNF4, HNF4a7, HNF4a8, | |
| | | HNF4a9, MODY, MODY1, | |
| | | NR2A1, NR2A21, TCF, | |
| | | TCF14, HNF4-alpha; hepatic | |
| | | nuclear factor 4 alpha; | |
| | | hepatocyte nuclear factor 4 | |
| | | alpha; transcription factor-14 | |
| 108 | haptoglobin | haptoglobin - hp2-alpha | HP |
| 109 | hydroxysteroid (11-beta) | Corticosteroid 11-beta- | HSD11B1 |
| | dehydrogenase 1 | dehydrogenase, isozyme 1; | |
| | | HDL; 11-DH; HSD11; | |
| | | HSD11B; HSD11L; 11-beta- | |
| | | HSD1 | |
| 110 | heat shock 70 kDa protein 1B | HSP70-2, heat shock 70 kD | HSPA1B |
| | | protein 1B | |
| 111 | islet amyloid polypeptide | Amylin - DAP, IAP, Islet | IAPP |
| | | amyloid polypeptide | |
| | | (Diabetes-associated peptide; | |
| | | amylin) | |
| 112 | intercellular adhesion molecule 1 | soluble intercellular adhesion | ICAM1 |
| | (CD54), human rhinovirus receptor | molecule-1, BB2, CD54, | |
| | | P3.58, 60 bp after segment 1; | |
| | | cell surface glycoprotein; cell | |
| | | surface glycoprotein P3.58; | |
| | | intercellular adhesion molecule 1 | |
| 113 | Intercellular adhesion molecule 3 | CD50, CDW50, ICAM-R | ICAM3 |
| | (CD50), | intercellular adhesion | |
| | | molecule-3 | |
| 114 | interferon, gamma | IFNG: IFG; IFI | IFNG |
| 115 | insulin-like growth factor 1 | IGF-1: somatomedin C. | IGF1 |
| | (somatomedin C) | insulin-like growth factor-1 | |
| 116 | insulin-like growth factor 2 | IGF-II polymorphisms | IGF2 |
| | (somatomedin A) | (somatomedin A) - C11orf43, | |
| | | INSIGF, pp9974, insulin-like | |
| | | growth factor 2; insulin-like | |
| | | growth factor II; insulin-like | |
| | | growth factor type 2; putative | |
| | | insulin-like growth factor II | |
| | | associated protein | |
| 117 | insulin-like growth factor binding | insulin-like growth factor | IGFBP1 |
| | protein 1 | binding protein-1 (IGFBP-1) - | |
| | | AFBP, IBP1, IGF-BP25, PP12, | |
| | | hIGFBP-1, IGF-binding | |
| | | protein 1; alpha-pregnancy- | |
| | | associated endometrial | |
| | | globulin; amniotic fluid | |
| | | binding protein; binding | |
| | | protein-25; binding protein-26; | |
| | | binding protein-28; growth | |
| | | hormone independent-binding | |
| | | protein; placental protein 12 | |
| 118 | insulin-like growth factor binding | insulin-like growth factor | IGFBP3 |
| | protein 3 | binding protein 3: IGF- | |
| | | binding protein 3 - BP-53, | |
| | | IBP3, IGF-binding protein 3; | |
| | | acid stable subunit of the 140 | |
| | | K IGF complex; binding | |
| | | protein 29; binding protein 53; | |
| | | growth hormone-dependent | |
| | | binding protein | |
| 119 | inhibitor of kappa light polypeptide | ikk-beta; IKK2; IKKB; | IKBKB |
| | gene enhancer in B-cells, kinase beta | NFKBIKB; IKK-beta; nuclear | |
| | | factor NF-kappa-B inhibitor | |
| | | kinase beta; inhibitor of | |
| | | nuclear factor kappa B kinase | |
| | | beta subunit | |
| 120 | interleukin 10 | IL-10, CSIF, IL-10, IL10A, | IL10 |
| | | TGIF, cytokine synthesis | |
| | | inhibitory factor | |
| 121 | interleukin 18 (interferon-gamma- | IL-18 - IGIF, IL-18, IL-1g, | IL18 |
| | inducing factor) | IL1F4, IL-1 gamma; | |
| | | interferon-gamma-inducing | |
| | | factor; interleukin 18; | |
| | | interleukin-1 gamma; | |
| | | interleukin-18 | |
| 122 | interleukin 1, alpha | IL 1 - IL-1A, IL1, IL1- | IL1A |
| | | ALPHA, IL1F1, IL1A | |
| | | (IL1F1); hematopoietin-1; | |
| | | preinterleukin 1 alpha; pro- | |
| | | interleukin-1-alpha | |
| 123 | interleukin 1, beta | interleukin-1 beta (IL-1 beta) - | IL1B |
| | | IL-1, IL1-BETA, IL1F2, | |
| | | catabolin; preinterleukin 1 | |
| | | beta; pro-interleukin-1-beta | |
| 124 | interleukin 1 receptor antagonist | interleukin-1 receptor | IL1RN |
| | | antagonist (IL-1Ra) - ICIL- | |
| | | 1RA, IL-1ra3, IL1F3, IL1RA, | |
| | | IRAP, IL1RIN (IL1F3); | |
| | | intracellular IL-1 receptor | |
| | | antagonist type II; intracellular | |
| | | interleukin-1 receptor | |
| | | antagonist (icIL-1ra); type II | |
| | | interleukin-1 receptor | |
| | | antagonist | |
| 125 | interleukin 2 | interleukin-2 (IL-2) - IL-2, | 1L2 |
| | | TCGF, lymphokine, T cell | |
| | | growth factor; aldesleukin; | |
| | | interleukin-2; involved in | |
| | | regulation of T-cell clonal | |
| | | expansion | |
| 126 | interleukin 2 receptor, alpha | Interleukin-2 receptor; IL- | IL2RA |
| | | 2RA; IL2RA; RP11-536K7.1; | |
| | | CD25; IDDM10; IL2R; | |
| | | TCGFR; interleukin 2 receptor, | |
| | | alpha chain | |
| 127 | interleukin 6 (interferon, beta 2) | Interleukin-6 (IL-6), BSF2, | IL6 |
| | | HGF, HSF, IFNB2, IL-6 | |
| 128 | interleukin 6 receptor | interleukin-6 receptor, soluble | IL6R |
| | | (sIL-6R) - CD126, IL-6R-1, | |
| | | IL-6R-alpha, IL6RA, CD126 | |
| | | antigen; interleukin 6 receptor | |
| | | alpha subunit | |
| 129 | interleukin 6 signal transducer | CD130, CDw130, GP130, | I16ST |
| | (gp130, oncostatin M receptor) | GP130-RAPS, IL6R-beta; | |
| | | CD130 antigen; IL6ST nirs | |
| | | variant 3; gp130 of the | |
| | | rheumatoid arthritis antigenic | |
| | | peptide-bearing soluble form; | |
| | | gp130 transducer chain; | |
| | | interleukin 6 signal transducer; | |
| | | interleukin receptor beta chain; | |
| | | membrane glycoprotein gp130; | |
| | | oncostatin M receptor | |
| 130 | interleukin 8 | Interleukin-8 (IL-8), 3-10C, | IL8 |
| | | AMCF-I, CXCL8, GCP-1, | |
| | | GCP1, IL-8, K60, LECT, | |
| | | LUCT, LYNAP, MDNCF, | |
| | | MONAP, NAF, NAP-1, | |
| | | NAP1, SCYB8, TSG-1, b- | |
| | | ENAP, CXC chemokine ligand | |
| | | 8; LUCT/interleukin-8; T cell | |
| | | chemotactic factor; beta- | |
| | | thromboglobulin-like protein; | |
| | | chemokine (C—X—C motif) | |
| | | ligand 8; emoctakin; | |
| | | granulocyte chemotactic | |
| | | protein 1; lymphocyte-derived | |
| | | neutrophil-activating factor; | |
| | | monocyte derived neutrophil- | |
| | | activating protein; monocyte- | |
| | | derived neutrophil chemotactic | |
| | | factor; neutrophil-activating | |
| | | factor; neutrophil-activating | |
| | | peptide 1; neutrophil-activating | |
| | | protein 1; protein 3-10C; small | |
| | | inducible cytokine subfamily | |
| | | B, member 8 | |
| 131 | inhibin, beta A (activin A, activin | activin A - EDF, FRP, Inhibin, | INHBA |
| | AB alpha polypeptide) | beta-1; inhibin beta A | |
| 132 | insulin | insulin, proinsulin | INS |
| 133 | insulin receptor | CD220, HHF5 | INSR |
| 134 | insulin promoter factor-1 | IPF-1, PDX-1 (pancreatic and | IPF1 |
| | | duodenal homeobox factor-1) | |
| 135 | insulin receptor substrate 1 | HIRS-1 | IRS1 |
| 136 | insulin receptor substrate-2 | IRS2 | IRS2 |
| 137 | potassium inwardly-rectifying | ATP gated K+ channels, Kir | KCNJ11 |
| | channel, subfamily J, member 11 | 6.2; BIR; HHF2; PHHI; | |
| | | IKATP; KIR6.2 | |
| 138 | potassium inwardly-rectifying | ATP gated K+ channels, Kir | KCNJ8 |
| | channel, subfamily J, member 8 | 6.1 | |
| 139 | klotho | klotho | KL |
| 140 | kallikrein B, plasma (Fletcher factor) 1 | kallikrein 3 - KLK3 - | KLKB1 |
| | | Kallikrein, plasma; kallikrein | |
| | | 3, plasma; kallikrein B plasma; | |
| | | kininogenin; plasma kallikrein | |
| | | B1 | |
| 141 | leptin (obesity homolog, mouse) | leptin - OB, OBS, leptin; leptin | LEP |
| | | (murine obesity homolog); | |
| | | obesity; obesity (murine | |
| | | homolog, leptin) | |
| 142 | leptin receptor | leptin receptor, soluble - | LEPR |
| | | CD295, OBR, OB receptor | |
| 143 | legumain | putative cysteine protease 1 - | LGMN |
| | | AEP, LGMN1, PRSC1, | |
| | | asparaginyl endopeptidase; | |
| | | cysteine protease 1; protease, | |
| | | cysteine, 1 (legumain) | |
| 144 | lipoprotein, Lp(a) | lipoprotein (a) [Lp(a)], AK38, | LPA |
| | | APOA, LP, Apolipoprotein | |
| | | Lp(a); antiangiogenic AK38 | |
| | | protein; apolipoprotein(a) | |
| 145 | lipoprotein lipase | LPL - LIPD | LPL |
| 146 | v-maf musculoaponeurotic | MafA (transcription factor) - | MAFA |
| | fibrosarcoma oncogene homolog A | RIPE3b1, hMafA, v-maf | |
| | (avian) | musculoaponeurotic | |
| | | fibrosarcoma oncogene | |
| | | homolog A | |
| 147 | mitogen-activated protein kinase 8 | IB1, JIP-1, JIP1, PRKM8IP, | MAPK8IP1 |
| | interacting protein 1 | JNK-interacting protein 1; | |
| | | PRKM8 interacting protein; | |
| | | islet-brain 1 | |
| 148 | mannose-binding lectin (protein C) | COLEC1, HSMBPC, MBL, | MBL2 |
| | 2, soluble (opsonic defect) | MBP, MBP1, Mannose- | |
| | | binding lectin 2, soluble | |
| | | (opsonic defect); mannan- | |
| | | binding lectin; mannan-binding | |
| | | protein; mannose binding | |
| | | protein; mannose-binding | |
| | | protein C; soluble mannose- | |
| | | binding lectin | |
| 149 | melanocortin 4 receptor | G protein coupled receptor | MC4R |
| | | MC4 | |
| 150 | melanin-concentrating hormone | G Protein-Coupled Receptor | MCHR1 |
| | receptor 1 | 24 - GPR24, MCH1R, SLC1, | |
| | | G protein-coupled receptor 24; | |
| | | G-protein coupled receptor 24 | |
| | | isoform 1, GPCR24 | |
| 151 | matrix metallopeptidase 12 | Matrix Metalloproteinases | MMP12 |
| | (macrophage elastase) | (MMP), HME, MME, | |
| | | macrophage elastase; | |
| | | macrophage metalloelastase; | |
| | | matrix metalloproteinase 12; | |
| | | matrix metalloproteinase 12 | |
| | | (macrophage elastase) | |
| 152 | matrix metallopeptidase 14 | Matrix Metalloproteinases | MMP14 |
| | (membrane-inserted) | (MMP), MMP-X1, MT1- | |
| | | MMP, MTMMP1, matrix | |
| | | metalloproteinase 14; matrix | |
| | | metalloproteinase 14 | |
| | | (membrane-inserted); | |
| | | membrane type 1 | |
| | | metalloprotease; membrane- | |
| | | type matrix metalloproteinase | |
| | | 1; membrane-type-1 matrix | |
| | | metalloproteinase | |
| 153 | matrix metallopeptidase 2 (gelatinase | Matrix Metalloproteinases | MMP2 |
| | A, 72 kDa gelatinase, 72 kDa type IV | (MMP), MMP-2, CLG4, | |
| | collagenase) | CLG4A, MMP-II, MONA, | |
| | | TBE-1, 72 kD type IV | |
| | | collagenase; collagenase type | |
| | | IV-A; matrix metalloproteinase | |
| | | 2; matrix metalloproteinase 2 | |
| | | (gelatinase A, 72 kD gelatinase, | |
| | | 72 kD type IV collagenase); | |
| | | matrix metalloproteinase 2 | |
| | | (gelatinase A, 72 kDa | |
| | | gelatinase, 72 kDa type IV | |
| | | collagenase); matrix | |
| | | metalloproteinase-II; | |
| | | neutrophil gelatinase | |
| 154 | matrix metallopeptidase 9 (gelatinase | Matrix Metalloproteinases | MMP9 |
| | B, 92 kDa gelatinase, 92 kDa type IV | (MMP), MMP-9, CLG4B, | |
| | collagenase) | GELB, 92 kD type IV | |
| | | collagenase; gelatinase B; | |
| | | macrophage gelatinase; matrix | |
| | | metalloproteinase 9; matrix | |
| | | metalloproteinase 9 (gelatinase | |
| | | B, 92 kD gelatinase, 92 kD type | |
| | | IV collagenase); matrix | |
| | | metalloproteinase 9 (gelatinase | |
| | | B, 92 kDa gelatinase, 92 kDa | |
| | | type IV collagenase); type V | |
| | | collagenase | |
| 155 | nuclear receptor co-repressor 1 | NCoR; thyroid hormone- and | NCOR1 |
| | | retinoic acid receptor- | |
| | | associated corepressor 1 | |
| 156 | neurogenic differentiation 1 | neuroD (transcription factor) - | NEUROD1 |
| | | BETA2, BHF-1, NEUROD | |
| 157 | nuclear factor of kappa light | nuclear factor, kappa B | NFKB1 |
| | polypeptide gene enhancer in B-cells | (NFKB); DNA binding factor | |
| | 1(p105) | KBF1; nuclear factor NF- | |
| | | kappa-B p50 subunit; nuclear | |
| | | factor kappa-B DNA binding | |
| | | subunit | |
| 158 | nerve growth factor, beta | B-type neurotrophic growth | NGFB |
| | polypeptide | factor (BNGF) - beta-nerve | |
| | | growth factor; nerve growth | |
| | | factor, beta subunit | |
| 159 | non-insulin-dependent Diabetes | NIDDM1 | NIDDM1 |
| | Mellitus (common, type 2) 1 | | |
| 160 | non-insulin-dependent Diabetes | NIDDM2 | NIDDM2 |
| | Mellitus (common, type 2) 2 | | |
| 161 | Noninsulin-dependent Diabetes | NIDDM3 | NIDDM3 |
| | Mellitus 3 | | |
| 162 | nischarin (imidazoline receptor) | imidazoline receptor; IRAS; I- | NISCH |
| | | 1 receptor candidate protein; | |
| | | imidazoline receptor candidate; | |
| | | imidazoline receptor antisera | |
| | | selected | |
| 163 | NF-kappaB repressing factor | NRF; ITBA4 gene; | NKRF |
| | | transcription factor NRF; NF- | |
| | | kappa B repressing factor; | |
| | | NF-kappa B-repressing factor | |
| 164 | neuronatin | Peg5 | NNAT |
| 165 | nitric oxide synthase 2A | NOS, type II; nitric oxide | NOS2A |
| | | synthase, macrophage | |
| 166 | Niemann-Pick disease, type C2 | epididymal secreting protein 1 - | NPC2 |
| | | HE1, NP-C2, epididymal | |
| | | secretory protein; epididymal | |
| | | secretory protein E1; tissue- | |
| | | specific secretory protein | |
| 167 | natriuretic peptide precursor B | B-type Natriuretic Peptide | NPPB |
| | | (BNP), BNP, brain type | |
| | | natriuretic peptide, pro-BNP?, | |
| | | NPPB | |
| 168 | nuclear receptor subfamily 1, group | Human Nuclear Receptor | NR1D1 |
| | D, member 1 | NR1D1 - EAR1, THRA1, | |
| | | THRAL, ear-1, hRev, Rev-erb- | |
| | | alpha; thyroid hormone | |
| | | receptor, alpha-like | |
| 169 | nuclear respiratory factor 1 | NRF1; ALPHA-PAL; alpha | NRF1 |
| | | palindromic-binding protein | |
| 170 | oxytocin, prepro-(neurophysin I) | oxytocin - OT, OT-NPI, | OXT |
| | | oxytocin-neurophysin I; | |
| | | oxytocin-neurophysin I, | |
| | | preproprotein | |
| 171 | purinergic receptor P2Y, G-protein | G Protein Coupled Receptor | P2RY10 |
| | coupled, 10 | P2Y10 - P2Y10, G-protein | |
| | | coupled purinergic receptor | |
| | | P2Y10; P2Y purinoceptor 10; | |
| | | P2Y-like receptor | |
| 172 | purinergic receptor P2Y, G-protein | G Protein-Coupled Receptor | P2RY12 |
| | coupled, 12 | P2Y12 - ADPG-R, HORK3, | |
| | | P2T(AC), P2Y(AC), | |
| | | P2Y(ADP), P2Y(cyc), P2Y12, | |
| | | SP1999, ADP-glucose | |
| | | receptor; G-protein coupled | |
| | | receptor SP1999; Gi-coupled | |
| | | ADP receptor HORK3; P2Y | |
| | | purinoceptor 12; platelet ADP | |
| | | receptor; purinergic receptor | |
| | | P2RY12; purinergic receptor | |
| | | P2Y, G-protein coupled 12; | |
| | | purinergic receptor P2Y12; | |
| | | putative G-protein coupled | |
| | | receptor | |
| 173 | purinergic receptor P2Y, G-protein | Purinoceptor 2 Type Y (P2Y2) - | P2RY2 |
| | coupled, 2 | HP2U, P2RU1, P2U, P2U1, | |
| | | P2UR, P2Y2, P2Y2R, ATP | |
| | | receptor; P2U nucleotide | |
| | | receptor; P2U purinoceptor 1; | |
| | | P2Y purinoceptor 2; purinergic | |
| | | receptor P2Y2; purinoceptor | |
| | | P2Y2 | |
| 174 | progestagen-associated endometrial | glycodelin-A; glycodelin- | PAEP |
| | protein (placental protein 14, | F; glycodelin- | |
| | pregnancy-associated endometrial | S; progesterone-associated | |
| | alpha-2-globulin, alpha uterine | endometrial protein | |
| | protein) | | |
| 175 | paired box gene 4 | Pax4 (transcription factor) - | PAX4 |
| | | paired domain gene 4 | |
| 176 | pre-B-cell colony enhancing factor 1 | visfatin; nicotinamide | PBEF1 |
| | | phosphoribosyltransferase | |
| 177 | phosphoenolpyruvate carboxykinase | PEPCK1; PEP carboxykinase; | PCK1 |
| | 1 (PEPCK1) | phosphopyruvate carboxylase; | |
| | | phosphoenolpyruvate | |
| | | carboxylase | |
| 178 | proprotein convertase | proprotein convertase 1 (PC1, | PCSK1 |
| | subtilisin/kexin type 1 | PC3, PCSK1, cleaves pro- | |
| | | insulin) | |
| 179 | placental growth factor, vascular | placental growth factor - | PGF |
| | endothelial growth factor-related | PLGF, PIGF-2 | |
| | protein | | |
| 180 | phosphoinositide-3-kinase, catalytic, | PI3K, p110-alpha, PI3-kinase | PIK3CA |
| | alpha polypeptide | p110 subunit alpha; PtdIns-3- | |
| | | kinase p110; | |
| | | phosphatidylinositol 3-kinase, | |
| | | catalytic, 110-KD, alpha; | |
| | | phosphatidylinositol 3-kinase, | |
| | | catalytic, alpha polypeptide; | |
| | | phosphatidylinositol-4,5- | |
| | | bisphosphate 3-kinase catalytic | |
| | | subunit, alpha isoform | |
| 181 | phosphoinositide-3-kinase, | phophatidylinositol 3-kinase; | PIK3R1 |
| | regulatory subunit 1 (p85 alpha) | phosphatidylinositol 3-kinase, | |
| | | regulatory, 1; | |
| | | phosphatidylinositol 3-kinase- | |
| | | associated p-85 alpha; | |
| | | phosphoinositide-3-kinase, | |
| | | regulatory subunit, polypeptide | |
| | | 1 (p85 alpha); | |
| | | phosphatidylinositol 3-kinase, | |
| | | regulatory subunit, polypeptide | |
| | | 1 (p85 alpha) | |
| 182 | phospholipase A2, group XIIA | PLA2G12, group XII secreted | PLA2G12A |
| | | phospholipase A2; group XIIA | |
| | | secreted phospholipase A2 | |
| 183 | phospholipase A2, group IID | phospholipase A2, secretory - | PLA2G2D |
| | | SPLASH, sPLA2S, secretory | |
| | | phospholipase A2s | |
| 184 | plasminogen activator, tissue | tissue Plasminogen Activator | PLAT |
| | | (tPA), T-PA, TPA, alteplase; | |
| | | plasminogen activator, tissue | |
| | | type; reteplase; t-plasminogen | |
| | | activator; tissue plasminogen | |
| | | activator (t-PA) | |
| 185 | patatin-like phospholipase domain | Adipose tissue lipase, ATGL - | PNPLA2 |
| | containing 2 | ATGL, TTS-2.2, adipose | |
| | | triglyceride lipase; desnutrin; | |
| | | transport-secretion protein 2.2; | |
| | | triglyceride hydrolase | |
| 186 | proopiomelanocortin | proopiomelanocortin - beta- | POMC |
| | (adrenocorticotropin/beta-lipotropin/ | LPH; beta-MSH; alpha-MSH; | |
| | alpha-melanocyte stimulating | gamma-LPH; gamma-MSH; | |
| | hormone/beta-melanocyte | corticotropin; beta-endorphin; | |
| | stimulating hormone/beta- | met-enkephalin; lipotropin | |
| | endorphin) | beta; lipotropin gamma; | |
| | | melanotropin beta; N-terminal | |
| | | peptide; melanotropin alpha; | |
| | | melanotropin gamma; pro- | |
| | | ACTH-endorphin; | |
| | | adrenocorticotropin; pro- | |
| | | opiomelanocortin; | |
| | | corticotropin-lipotrophin; | |
| | | adrenocorticotropic hormone; | |
| | | alpha-melanocyte-stimulating | |
| | | hormone; corticotropin-like | |
| | | intermediary peptide | |
| 187 | paraoxonase 1 ESA, PON, | paraoxonase - ESA, PON, | PON1 |
| | Paraoxonase | Paraoxonase | |
| 188 | peroxisome proliferative activated | Peroxisome proliferator- | PPARA |
| | receptor, alpha | activated receptor (PPAR), | |
| | | NR1C1, PPAR, hPPAR, PPAR | |
| | | alpha | |
| 189 | peroxisome proliferative activated | Peroxisome proliferator- | PPARD |
| | receptor, delta | activated receptor (PPAR), | |
| | | FAAR, NR1C2, NUC1, NUCI, | |
| | | NUCII, PPAR-beta, PPARB, | |
| | | nuclear hormone receptor 1, | |
| | | PPAR Delta | |
| 190 | peroxisome proliferative activated | Peroxisome proliferator- | PPARG |
| | receptor, gamma | activated receptor (PPAR), | |
| | | HUMPPARG, NR1C3, | |
| | | PPARG1, PPARG2, PPAR | |
| | | gamma; peroxisome | |
| | | proliferative activated receptor | |
| | | gamma; peroxisome | |
| | | proliferator activated-receptor | |
| | | gamma; peroxisome | |
| | | proliferator-activated receptor | |
| | | gamma 1; ppar gamma2 | |
| 191 | peroxisome proliferative activated | Pgc1 alpha; PPAR gamma | PPARGC1A |
| | receptor, gamma, coactivator 1 | coactivator-1; ligand effect | |
| | | modulator-6; PPAR gamma | |
| | | coactivator variant form3 | |
| 192 | protein phosphatase 1, regulatory | PP1G, PPP1R3, protein | PPP1R3A |
| | (inhibitor) subunit 3A (glycogen and | phosphatase 1 glycogen- | |
| | sarcoplasmic reticulum binding | associated regulatory subunit; | |
| | subunit, skeletal muscle) | protein phosphatase 1 | |
| | | glycogen-binding regulatory | |
| | | subunit 3; protein phosphatase | |
| | | type-1 glycogen targeting | |
| | | subunit; serine/threonine | |
| | | specific protein phosphatase; | |
| | | type-1 protein phosphatase | |
| | | skeletal muscle glycogen | |
| | | targeting subunit | |
| 193 | protein phosphatase 2A, regulatory | protein phosphatase 2A - | PPP2R4 |
| | subunit B' (PR 53) | PP2A, PR53, PTPA, PP2A, | |
| | | subunit B'; phosphotyrosyl | |
| | | phosphatase activator; protein | |
| | | phosphatase 2A, regulatory | |
| | | subunit B' | |
| 194 | protein kinase, AMP-activated, beta | on list as adenosine | PRKAB1 |
| | 1 non-catalytic subunit | monophosphate kinase? - | |
| | | AMPK, HAMPKb, 5′-AMP- | |
| | | activated protein kinase beta-1 | |
| | | subunit; AMP-activated | |
| | | protein kinase beta 1 non- | |
| | | catalytic subunit; AMP- | |
| | | activated protein kinase beta | |
| | | subunit; AMPK beta-1 chain; | |
| | | AMPK beta 1; protein kinase, | |
| | | AMP-activated, noncatalytic, | |
| | | beta-1 | |
| 195 | protein kinase, cAMP-dependent, | PKA (kinase) - PKACA, PKA | PRKACA |
| | catalytic, alpha | C-alpha; cAMP-dependent | |
| | | protein kinase catalytic subunit | |
| | | alpha; cAMP-dependent | |
| | | protein kinase catalytic subunit | |
| | | alpha, isoform 1; protein | |
| | | kinase A catalytic subunit | |
| 196 | protein kinase C, epsilon | PKC-epsilon - PKCE, nPKC- | PRKCE |
| | | epsilon | |
| 197 | proteasome (prosome, macropain) | Bridge-1; homolog of rat | PSMD9 |
| | 26S subunit, non-ATPase, 9 (Bridge- | Bridge 1; 26S proteasome | |
| | 1) | regulatory subunit p27; | |
| | | proteasome 26S non-ATPase | |
| | | regulatory subunit 9 | |
| 198 | prostaglandin E synthase | mPGES - MGST-IV, MGST1- | PTGES |
| | | L1, MGST1L1, PGES, PIG12, | |
| | | PP102, PP1294, TP5I12 | |
| | | Other Designations: MGST1- | |
| | | like 1; glutathione S- | |
| | | transferase 1-like 1; | |
| | | microsomal glutathione S- | |
| | | transferase 1-like 1; p53- | |
| | | induced apoptosis protein 12; | |
| | | p53-induced gene 12; tumor | |
| | | protein p53 inducible protein | |
| | | 12 | |
| 199 | prostaglandin-endoperoxide synthase | Cyclo-oxygenase-2 (COX-2) - | PTGS2 |
| | 2 (prostaglandin G/H synthase and | COX-2, COX2, PGG/HS, | |
| | cyclooxygenase) | PGHS-2, PHS-2, hCox-2, | |
| | | cyclooxygenase 2b; | |
| | | prostaglandin G/H synthase | |
| | | and cyclooxygenase; | |
| | | prostaglandin-endoperoxide | |
| | | synthase 2 | |
| 200 | protein tyrosine phosphatase, | PTPMT1 - PLIP, PNAS-129, | PTPMT1 |
| | mitochondrial 1 | NB4 apoptosis/differentiation | |
| | | related protein; PTEN-like | |
| | | phosphatase | |
| 201 | Peptide YY | PYY1 | PYY |
| 202 | retinol binding protein 4, plasma | RBP4; retinol-binding protein | RBP4 |
| | (RBP4) | 4, plasma; retinol-binding | |
| | | protein 4, interstitial | |
| 203 | regenerating islet-derived 1 alpha | regenerating gene product | REG1A |
| | (pancreatic stone protein, pancreatic | (Reg); protein-X; lithostathine | |
| | thread protein) | 1 alpha; pancreatic thread | |
| | | protein; regenerating protein I | |
| | | alpha; islet cells regeneration | |
| | | factor; pancreatic stone | |
| | | protein, secretory; islet of | |
| | | langerhans regenerating | |
| | | potein | |
| 204 | resistin | resistin - ADSF, FIZZ3, | RETN |
| | | RETN1, RSTN, XCP1, | |
| | | C/EBP-epsilon regulated | |
| | | myeloid-specific secreted | |
| | | cysteine-rich protein precursor | |
| | | 1; found in inflammatory zone 3 | |
| 205 | ribosomal protein S6 kinase, 90 kDa, | S6-kinase 1 - HU-1, RSK, | RPS6KA1 |
| | polypeptide 1 | RSK1, S6K-alpha 1, | |
| | | (ribosomal protein S6 kinase, | |
| | | 90 kD, polypeptide 1); p90- | |
| | | RSK 1; ribosomal protein S6 | |
| | | kinase alpha 1; ribosomal | |
| | | protein S6 kinase, 90 kD, 1; | |
| | | ribosomal protein S6 kinase, | |
| | | 90 kD, polypeptide 1 | |
| 206 | Ras-related associated with Diabetes | RAD, RAD1, REM3, RAS | RRAD |
| | | (RAD and GEM) like GTP | |
| | | binding 3 | |
| 207 | serum amyloid A1 | Serum Amyloid A (SAA), | SAA1 |
| | | PIG4, SAA, TP53I4, tumor | |
| | | protein p53 inducible protein 4 | |
| 208 | selectin E (endothelial adhesion | E-selectin, CD62E, ELAM, | SELE |
| | molecule 1) | ELAM1, ESEL, LECAM2, | |
| | | leukocyte endothelial cell | |
| | | adhesion molecule 2; selectin | |
| | | E, endothelial adhesion | |
| | | molecule 1 | |
| 209 | selectin P (granule membrane | CD62, CD62P, FLJ45155, | SELP |
| | protein 140 kDa, antigen CD62) | GMP140, GRMP, PADGEM, | |
| | | PSEL; antigen CD62; | |
| | | granulocyte membrane protein; | |
| | | selectin P; selectin P (granule | |
| | | membrane protein 140 kD, | |
| | | antigen CD62) | |
| 210 | serpin peptidase inhibitor, clade A | corticosteroid-binding | SERPINA6 |
| | (alpha-1 antiproteinase, antitrypsin), | globulin; transcortin; | |
| | member 6 | corticosteroid binding | |
| | | globulin; serine (or cysteine) | |
| | | proteinase inhibitor, clade A | |
| | | (alpha-1 antiproteinase, | |
| | | antitrypsin), member 6 | |
| 211 | serpin peptidase inhibitor, clade E | plasminogen activator | SERPINE1 |
| | (nexin, plasminogen activator | inhibitor-1 - PAI, PAI-I, PAI1, | |
| | inhibitor type 1), member 1 | PLANH1, plasminogen | |
| | | activator inhibitor, type I; | |
| | | plasminogen activator | |
| | | inhibitor-1; serine (or cysteine) | |
| | | proteinase inhibitor, clade E | |
| | | (nexin, plasminogen activator | |
| | | inhibitor type 1), member 1 | |
| 212 | serum/glucocorticoid regulated | Serum/Glucocorticoid | SGK |
| | kinase | Regulated Kinase 1 - SGK1, | |
| | | serine/threonine protein kinase | |
| | | SGK; serum and | |
| | | glucocorticoid regulated kinase | |
| 213 | sex hormone-binding globulin | sex hormone-binding globulin | SHBG |
| | | (SHBG) - ABP, Sex hormone- | |
| | | binding globulin (androgen | |
| | | binding protein) | |
| 214 | thioredoxin interacting protein | Sirt1; SIR2alpha; sir2-like 1; | SIRT1 |
| | | sirtuin type 1; sirtuin (silent | |
| | | mating type information | |
| | | regulation 2, S. cerevisiae, | |
| | | homolog) 1 | |
| 215 | solute carrier family 2, member 10 | glucose transporter 10 | SLC2A10 |
| | | (GLUT10); ATS | |
| 216 | solute carrier family 2, member 2 | glucose transporter 2 | SLC2A2 |
| | | (GLUT2) | |
| 217 | solute carrier family 2, member 4 | glucose transporter 4 | SLC2A4 |
| | | (GLUT4) | |
| 218 | solute carrier family 7 (cationic | ERR - ATRC1, CAT-1, ERR, | SLC7A1 |
| | amino acid transporter, y+ system), | HCAT1, REC1L, amino acid | |
| | member 1(ERR) | transporter, cationic 1; | |
| | | ecotropic retroviral receptor | |
| 219 | SNF1-like kinase 2 | Sik2; salt-inducible kinase 2; | SNF1LK2 |
| | | salt-inducible serine/threonine | |
| | | kinase 2 | |
| 220 | suppressor of cytokine signaling 3 | CIS3, Cish3, SOCS-3, SSI-3, | SOCS3 |
| | | SSI3, STAT induced STAT | |
| | | inhibitor 3; cytokine-induced | |
| | | SH2 protein 3 | |
| 221 | v-src sarcoma (Schmidt-Ruppin A-2) | ASV, SRC1, c-SRC, p60-Src, | SRC |
| | viral oncogene homolog (avian) | proto-oncogene tyrosine- | |
| | | protein kinase SRC; | |
| | | protooncogene SRC, Rous | |
| | | sarcoma; tyrosine kinase | |
| | | pp60c-src; tyrosine-protein | |
| | | kinase SRC-1 | |
| 222 | sterol regulatory element binding | sterol regulatory element- | SREBF1 |
| | transcription factor 1 | binding protein 1c (SREBP-1c) | |
| 223 | solute carrier family 2, member 4 | SMST, somatostatin-14, | SST |
| | | somatostatin-28 | |
| 224 | somatostatin receptor 2 | somatostatin receptor subtype 2 | SSTR2 |
| 225 | somatostatin receptor 5 | somatostatin receptor 5 - | SSTR5 |
| | | somatostatin receptor subtype 5 | |
| 226 | transcription factor 1, hepatic; LF- | HNF1α; albumin proximal | TCF1 |
| | B1, hepatic nuclear factor (HNF1) | factor; hepatic nuclear factor 1; | |
| | | maturity onset Diabetes of the | |
| | | young 3; Interferon production | |
| | | regulator factor (HNF1) | |
| 227 | transcription factor 2, hepatic; LF- | hepatocyte nuclear factor 2 - | TCF2 |
| | B3; variant hepatic nuclear factor | FJHN, HNF1B, HNF1beta, | |
| | | HNF2, LFB3, MODY5, | |
| | | VHNF1, transcription factor 2 | |
| 228 | transcription factor 7-like 2 (T-cell | TCF7L2 - TCF-4, TCF4 | TCF7L2 |
| | specific, HMG-box) | | |
| 229 | transforming growth factor, beta 1 | TGF-beta: TGF-beta 1 protein; | TGFB1 |
| | (Camurati-Engelmann disease) | diaphyseal dysplasia 1, | |
| | | progressive; transforming | |
| | | growth factor beta 1; | |
| | | transforming growth factor, | |
| | | beta 1; transforming growth | |
| | | factor-beta 1, CED, DPD1, | |
| | | TGFB | |
| 230 | transglutaminase 2 (C polypeptide, | TG2, TGC, C polypeptide; | TGM2 |
| | protein-glutamine-gamma- | TGase C; TGase-H; protein- | |
| | glutamyltransferase) | glutamine-gamma- | |
| | | glutamyltransferase; tissue | |
| | | transglutaminase; | |
| | | transglutaminase 2; | |
| | | transglutaminase C | |
| 231 | thrombospondin 1 | thrombospondin - THBS, TSP, | THBS1 |
| | | TSP1, thrombospondin-1p180 | |
| 232 | thrombospondin, type I, domain | TMTSP, UNQ3010, | THSD1 |
| | containing 1 | thrombospondin type I | |
| | | domain-containing 1; | |
| | | thrombospondin, type I, | |
| | | domain 1; transmembrane | |
| | | molecule with thrombospondin | |
| | | module | |
| 233 | TIMP metallopeptidase inhibitor | CSC-21K; tissue inhibitor of | TIMP2 |
| | | metalloproteinase 2; tissue | |
| | | inhibitor of metalloproteinase | |
| | | 2 precursor; tissue inhibitor of | |
| | | metalloproteinases 2 | |
| 234 | tumor necrosis factor (TNF | TNF-alpha (tumour necrosis | TNF |
| | superfamily, member 2) | factor-alpha) - DIF, TNF- | |
| | | alpha, TNFA, TNFSF2, APC1 | |
| | | protein; TNF superfamily, | |
| | | member 2; TNF, macrophage- | |
| | | derived; TNF, monocyte- | |
| | | derived; cachectin; tumor | |
| | | necrosis factor alpha | |
| 235 | tumor necrosis factor receptor | MGC29565, OCIF, OPG, TR1; | TNFRSF11B |
| | superfamily, member 11b | osteoclastogenesis inhibitory | |
| | (osteoprotegerin) | factor; osteoprotegerin | |
| 236 | tumor necrosis factor receptor | tumor necrosis factor receptor | TNFRSF1A |
| | superfamily, member 1A | 1 gene R92Q polymorphism - | |
| | | CD120a, FPF, TBP1, TNF-R, | |
| | | TNF-R-I, TNF-R55, TNFAR, | |
| | | TNFR1, TNFR55, TNFR60, | |
| | | p55, p55-R, p60, tumor | |
| | | necrosis factor binding protein | |
| | | 1; tumor necrosis factor | |
| | | receptor 1; tumor necrosis | |
| | | factor receptor type 1; tumor | |
| | | necrosis factor-alpha receptor | |
| 237 | tumor necrosis factor receptor | soluble necrosis factor receptor - | TNFRSF1B |
| | superfamily, member 1B | CD120b, TBPII, TNF-R-II, | |
| | | TNF-R75, TNFBR, TNFR2, | |
| | | TNFR80, p75, p75TNFR, p75 | |
| | | TNF receptor; tumor necrosis | |
| | | factor beta receptor; tumor | |
| | | necrosis factor binding protein | |
| | | 2; tumor necrosis factor | |
| | | receptor 2 | |
| 238 | tryptophan hydroxylase 2 | enzyme synthesizing serotonin; | TPH2 |
| | | neuronal tryptophan | |
| | | hydroxylase, NTPH | |
| 239 | thyrotropin-releasing hormone | thyrotropin-releasing hormone | TRH |
| 240 | transient receptor potential cation | vanilloid receptor 1 - VR1, | TRPV1 |
| | channel, subfamily V, member 1 | capsaicin receptor; transient | |
| | | receptor potential vanilloid 1a; | |
| | | transient receptor potential | |
| | | vanilloid 1b; vanilloid receptor | |
| | | subtype 1, capsaicin receptor; | |
| | | transient receptor potential | |
| | | vanilloid subfamily 1 (TRPV1) | |
| 241 | thioredoxin interacting protein | thioredoxin binding protein 2; | TXNIP |
| | | upregulated by 1,25- | |
| | | dihydroxyvitamin D-3 | |
| 242 | thioredoxin reductase 2 | TR; TR3; SELZ; TRXR2; TR- | TXNRD2 |
| | | BETA; selenoprotein Z; | |
| | | thioredoxin reductase 3; | |
| | | thioredoxin reductase beta | |
| 243 | urocortin 3 (stresscopin) | archipelin, urocortin III, SCP, | UCN3 |
| | | SPC, UGNIII, stresscopin; | |
| | | urocortin 3 | |
| 244 | uncoupling protein 2 (mitochondrial, | UCPH, uncoupling protein 2; | UCP2 |
| | proton carrier) | uncoupling protein-2 | |
| 245 | upstream transcription factor 1 | major late transcription factor 1 | USF1 |
| 246 | urotensin 2 | PRO1068, U-II, UCN2, UII | UTS2 |
| 247 | vascular cell adhesion molecule 1 | (soluble) vascular cell | VCAM1 |
| | | adhesion molecule-1, CD106, | |
| | | INCAM-100, CD106 antigen, | |
| | | VCAM-1 | |
| 248 | vascular endothelial growth factor | VEGF - VEGFA, VPF, | VEGF |
| | | vascular endothelial growth | |
| | | factor A; vascular permeability | |
| | | factor | |
| 249 | vimentin | vimentin | VIM |
| 250 | vasoactive intestinal peptide | vasoactive intestinal peptide - | VIP |
| | | PHM27 | |
| 251 | vasoactive intestinal peptide receptor 1 | vasoactive intestinal peptide | VIPR1 |
| | | receptor 1 - HVR1, II, | |
| | | PACAP-R-2, RCD1, RDC1, | |
| | | VIPR, VIRG, VPAC1, PACAP | |
| | | type II receptor; VIP receptor, | |
| | | type I; pituitary adenylate | |
| | | cyclase activating polypeptide | |
| | | receptor, type II | |
| 252 | vasoactive intestinal peptide receptor 2 | Vasoactive Intestinal Peptide | VIPR2 |
| | | Receptor 2 - VPAC2 | |
| 253 | von Willebrand factor | von Willebrand factor, | VWF |
| | | F8VWF, VWD, coagulation | |
| | | factor VIII VWF | |
| 254 | Wolfram syndrome 1 (wolframin) | DFNA14, DFNA38, DFNA6, | WFS1 |
| | | DIDMOAD, WFRS, WFS, | |
| | | WOLFRAMIN | |
| 255 | X-ray repair complementing | Ku autoantigen, 70 kDa; Ku | XRCC6 |
| | defective repair in Chinese hamster | autoantigen p70 subunit; | |
| | cells 6 | thyroid-lupus autoantigen p70; | |
| | | CTC box binding factor 75 kDa | |
| | | subunit; thyroid | |
| | | autoantigen 70 kD (Ku | |
| | | antigen); thyroid autoantigen | |
| | | 70 kDa (Ku antigen); ATP- | |
| | | dependent DNA helicase II, 70 kDa | |
| | | subunit | |
| 256 | c-peptide | c-peptide, soluble c-peptide | |
| 257 | cortisol | cortisol - hydrocortisone is the | |
| | | synthetic form | |
| 258 | vitamin D3 | vitamin D3 | |
| 259 | estrogen | estrogen | |
| 260 | estradiol | estradiol | |
| 261 | digitalis-like factor | digitalis-like factor | |
| 262 | oxyntomodulin | oxyntomodulin | |
| 263 | dehydroepiandrosterone sulfate | dehydroepiandrosterone sulfate | |
| | (DHEAS) | (DHEAS) | |
| 264 | serotonin (5-hydroxytryptamine) | serotonin (5- | |
| | | hydroxytryptamine) | |
| 265 | anti-CD38 autoantibodies | anti-CD38 autoantibodies | |
| 266 | gad65 autoantibody | gad65 autoantibody epitopes | |

One skilled in the art will note that the above listed T2DMARKERS come from a diverse set of physiological and biological pathways, including many which are not commonly accepted to be related to Diabetes. These groupings of different T2DMARKERS, even within those high significance segments, may presage differing signals of the stage or rate of the progression of the disease. Such distinct groupings of T2DMARKERS may allow a more biologically detailed and clinically useful signal from the T2DMARKERS as well as opportunities for pattern recognition within the T2MARKER algorithms combining the multiple T2DMARKER signals.

The present invention concerns, in one aspect, a subset of T2DMARKERS; other T2DMARKERS and even biomarkers which are not listed in the above Table 1, but related to these physiological and biological pathways, may prove to be useful given the signal and information provided from these studies. To the extent that other biomarker pathway participants (i.e., other biomarker participants in common pathways with those biomarkers contained within the list of T2DMARKERS in the above Table 1) are also relevant pathway participants in pre-Diabetes, Diabetes, or a pre-diabetic condition, they may be functional equivalents to the biomarkers thus far disclosed in Table 1. These other pathway participants are also considered T2DMARKERS in the context of the present invention, provided they additionally share certain defined characteristics of a good biomarker, which would include both involvement in the herein disclosed biological processes and also analytically important characteristics such as the bioavailability of said biomarkers at a useful signal to noise ratio, and in a useful sample matrix such as blood serum. Such requirements typically limit the diagnostic usefulness of many members of a biological pathway, and frequently occurs only in pathway members that constitute secretory substances, those accessible on the plasma membranes of cells, as well as those that are released into the serum upon cell death, due to apoptosis or for other reasons such as endothelial remodeling or other cell turnover or cell necrotic processes, whether or not they are related to the disease progression of pre-Diabetes, a pre-diabetic condition, and Diabetes. However, the remaining and future biomarkers that meet this high standard for T2DMARKERS are likely to be quite valuable. Furthermore, other unlisted biomarkers will be very highly correlated with the biomarkers listed as T2DMARKERS in Table 1 (for the purpose of this application, any two variables will be considered to be “very highly correlated” when they have a Coefficient of Determination (R²) of 0.5 or greater). The present invention encompasses such functional and statistical equivalents to the aforementioned T2DMARKERS. Furthermore, the statistical utility of such additional T2DMARKERS is substantially dependent on the cross-correlation between multiple biomarkers and any new biomarkers will often be required to operate within a panel in order to elaborate the meaning of the underlying biology.

One or more, preferably two or more of the listed T2DMARKERS can be detected in the practice of the present invention. For example, two (2), three (3), four (4), five (5), ten (10), fifteen (15), twenty (20), forty (40), fifty (50), seventy-five (75), one hundred (100), one hundred and twenty five (125), one hundred and fifty (150), one hundred and seventy-five (175), two hundred (200), two hundred and ten (210), two hundred and twenty (220), two hundred and thirty (230), two hundred and forty (240), two hundred and fifty (250), two hundred and sixty (260) or more T2DMARKERS can be detected. In some aspects, all 266 T2DMARKERS listed herein can be detected. Preferred ranges from which the number of T2DMARKERS can be detected include ranges bounded by any minimum selected from between one and 266, particularly two, five, ten, twenty, fifty, seventy-five, one hundred, one hundred and twenty five, one hundred and fifty, one hundred and seventy-five, two hundred, two hundred and ten, two hundred and twenty, two hundred and thirty, two hundred and forty, two hundred and fifty, paired with any maximum up to the total known T2DMARKERS, particularly five, ten, twenty, fifty, and seventy-five. Particularly preferred ranges include two to five (2-5), two to ten (2-10), two to fifty (2-50), two to seventy-five (2-75), two to one hundred (2-100), five to ten (5-10), five to twenty (5-20), five to fifty (5-50), five to seventy-five (5-75), five to one hundred (5-100), ten to twenty (10-20), ten to fifty (10-50), ten to seventy-five (10-75), ten to one hundred (10-100), twenty to fifty (20-50), twenty to seventy-five (20-75), twenty to one hundred (20-100), fifty to seventy-five (50-75), fifty to one hundred (50-100), one hundred to one hundred and twenty-five (100-125), one hundred and twenty-five to one hundred and fifty (125-150), one hundred and fifty to one hundred and seventy five (150-175), one hundred and seventy-five to two hundred (175-200), two hundred to two hundred and ten (200-210), two hundred and ten to two hundred and twenty (210-220), two hundred and twenty to two hundred and thirty (220-230), two hundred and thirty to two hundred and forty (230-240), two hundred and forty to two hundred and fifty (240-250), two hundred and fifty to two hundred and sixty (250-260), and two hundred and sixty to more than two hundred and sixty (260+).

Construction of T2DMARKER Panels

Groupings of T2DMARKERS can be included in “panels.” A “panel” within the context of the present invention means a group of biomarkers (whether they are T2DMARKERS, clinical parameters, or traditional laboratory risk factors) that includes more than one T2DMARKER. A panel can also comprise additional biomarkers, e.g., clinical parameters, traditional laboratory risk factors, known to be present or associated with Diabetes, in combination with a selected group of the T2DMARKERS listed in Table 1.

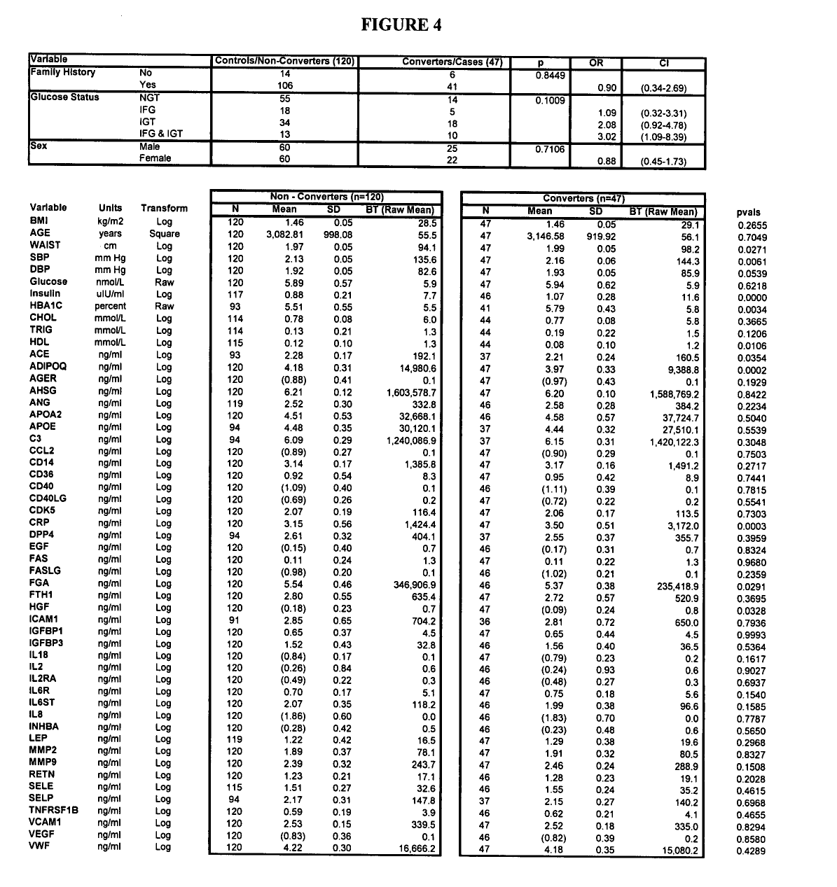

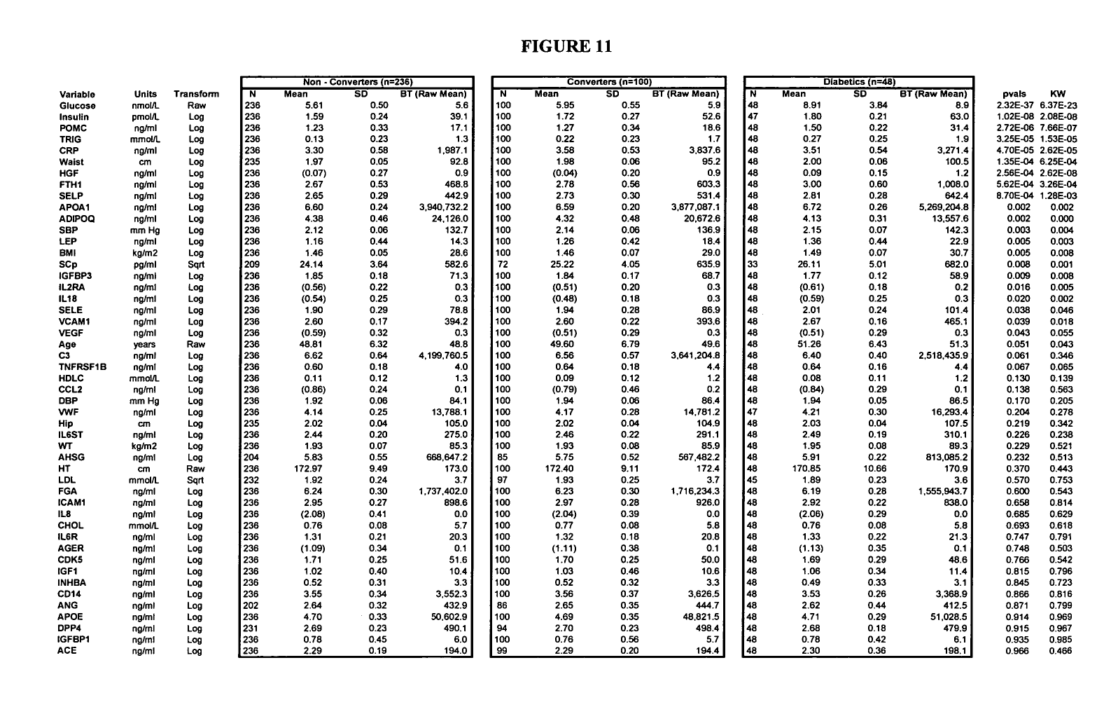

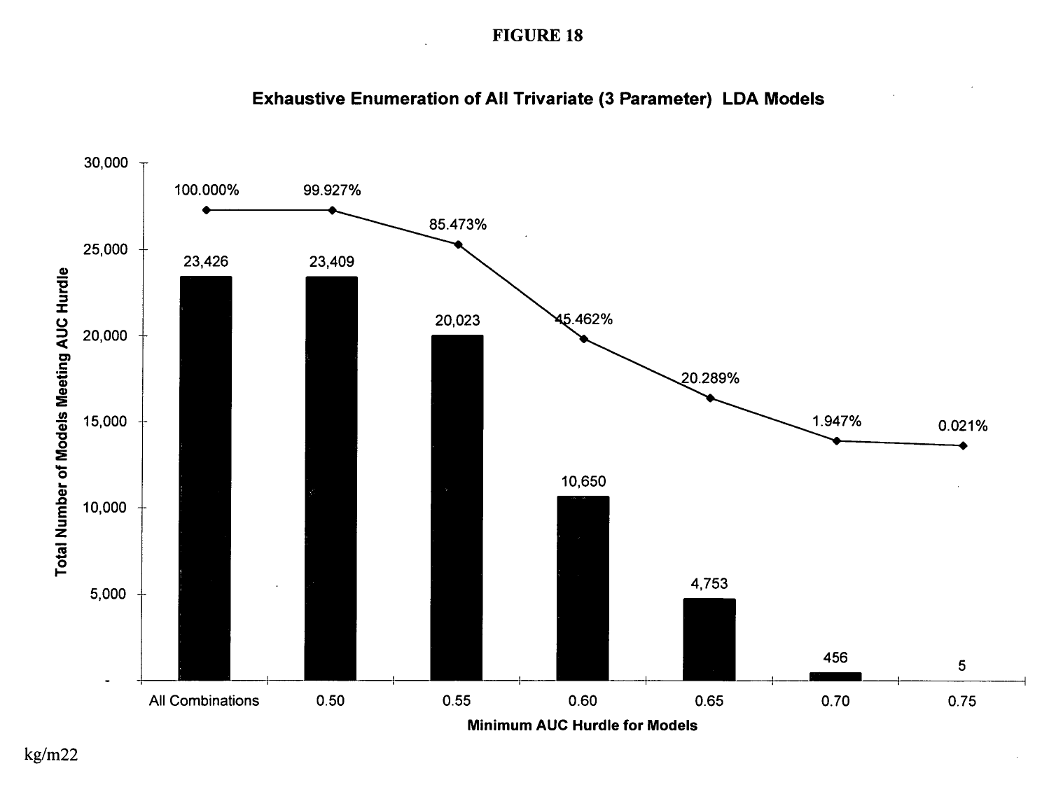

As noted above, many of the individual T2DMARKERS, clinical parameters, and traditional laboratory risk factors listed, when used alone and not as a member of a multi-biomarker panel of T2DMARKERS, have little or no clinical use in reliably distinguishing individual normal (or “normoglycemic”), pre-Diabetes, and Diabetes subjects from each other in a selected general population, and thus cannot reliably be used alone in classifying any patient between those three states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed. As discussed above, in the study populations of the below Examples, none of the individual T2DMARKERS demonstrated a very high degree of diagnostic accuracy when used by itself for the diagnosis of pre-Diabetes, even though many showed statistically significant differences between the three subject populations (as seen in FIG. 4 and FIG. 11 in the relevant Example 1 and 2 populations). However, when each T2DMARKER is taken individually to assess the individual subjects of the population, such T2DMARKERS are of limited use in the intended risk indications for the invention (as is shown in FIG. 17 and 18). The few exceptions to this were generally in their use distinguishing frank Diabetes from normal, where several of the biomarkers (for example, glucose, insulin, HBA1c) are part of the clinical definition and symptomatic pathology of Diabetes itself.

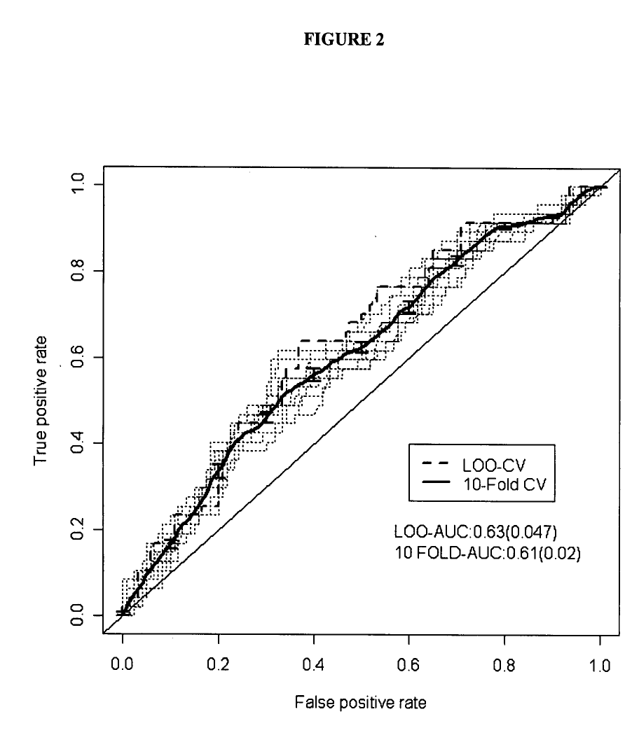

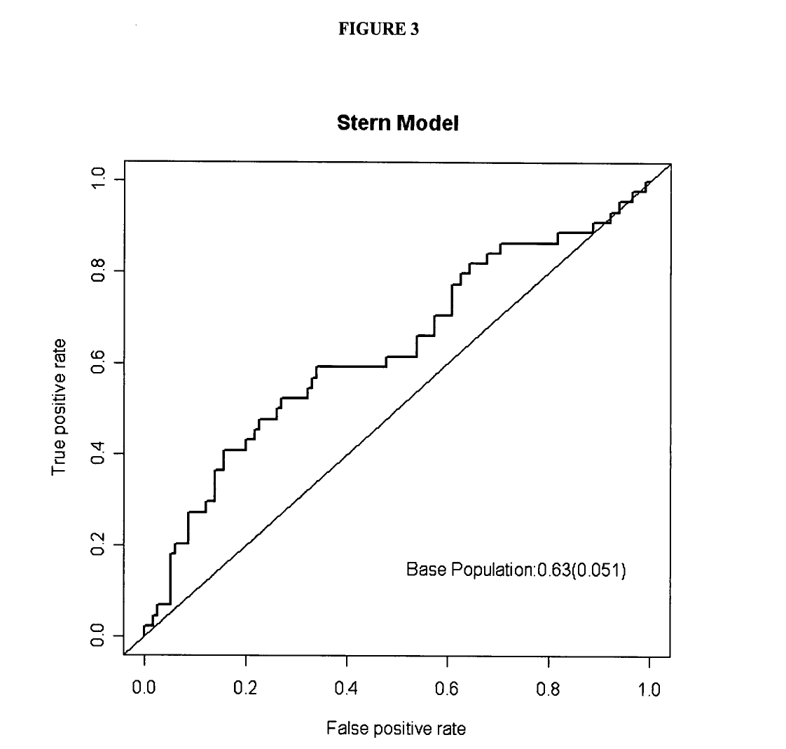

Combinations of multiple clinical parameters used singly alone or together in formulas is another approach, but also generally has difficulty in reliably achieving a high degree of diagnostic accuracy for individual subjects when tested across multiple study populations except when the blood-borne biomarkers are included (by way of example, FIG. 2 demonstrates this in the Base population of Example 1). Even when individual traditional laboratory risk factors that are blood-borne biomarkers are added to clinical parameters, as with glucose and HDLC within the Diabetes risk index of Stern (2002), it is difficult to reliably achieve a high degree of diagnostic accuracy for individual subjects when tested across multiple study populations (by way of example, FIG. 3 demonstrates this in the Base population of Example 1). Used herein, for a formula or biomarker (including T2DMARKERS, clinical parameters, and traditional laboratory risk factors) to “reliably achieve” a given level of diagnostic accuracy measnt to achieve this metric under cross-validation (such as LOO-CV or 10-Fold CV within the original population) or in more than one population (e.g., demonstrate it beyond the original population in which the formula or biomarker was originally measured and trained). It is recognized that biological variability is such that it is unlikely that any given formula or biomarker will achieve the same level of diagnostic accuracy in every individual population in which it can be measured, and that substantial similarity between such training and validation populations is assumed and, indeed, required.

Despite this individual T2DMARKER performance, and the general performance of formulas combining only the traditional clinical parameters and few traditional laboratory risk factors, the present inventors have noted that certain specific combinations of two or more T2DMARKERS can also be used as multi-biomarker panels comprising combinations of T2DMARKERS that are known to be involved in one or more physiological or biological pathways, and that such information can be combined and made clinically useful through the use of various formulae, including statistical classification algorithms and others, combining and in many cases extending the performance characteristics of the combination beyond that of the individual T2DMARKERS. These specific combinations show an acceptable level of diagnostic accuracy, and, when sufficient information from multiple T2DMARKERS is combined in a trained formula, often reliably achieve a high level of diagnostic accuracy transportable from one population to another.

The general concept of how two less specific or lower performing T2DMARKERS are combined into novel and more useful combinations for theintended indications, is a key aspect of the invention. Multiple biomarkers can often yield better performance than the individual components when proper mathematical and clinical algorithms are used; this is often evident in both sensitivity and specificity, and results in a greater AUC. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

Several statistical and modeling algorithms known in the art can be used to both assist in T2DMARKER selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the T2DMARKERS can be advantageously used. While such grouping may or may not give direct insight into the biology and desired informational content targets for ideal pre-Diabetes formula, it is the result of a method of factor analysis intended to group collections of T2DMARKERS with similar information content (see Examples below for more statistical techniques commonly employed). Pathway informed seeding of such statistical classification techniques also may be employed, as may rational approaches based on the selection of individual T2DMARKERS based on their participation across in particular pathways or physiological functions.

Ultimately, formula such as statistical classification algorithms can be directly used to both select T2DMARKERS and to generate and train the optimal formula necessary to combine the results from multiple T2DMARKERS into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of T2DMARKERS used. The position of the individual T2DMARKER on a forward or backwards selected panel can be closely related to its provision of incremental information content for the algorithm, so the order of contribution is highly dependent on the other constituent T2DMARKERS in the panel.

The inventors have observed that certain T2DMARKERS are frequently selected across many different formulas and model types for biomarker selection and model formula construction. One aspect of the present invention relates to selected key biomarkers that are categorized based on the frequency of the presence of the T2DMARKERS and in the best fit models of given types taken across multiple population studies, such as those shown in Examples 1 and 2 herein.

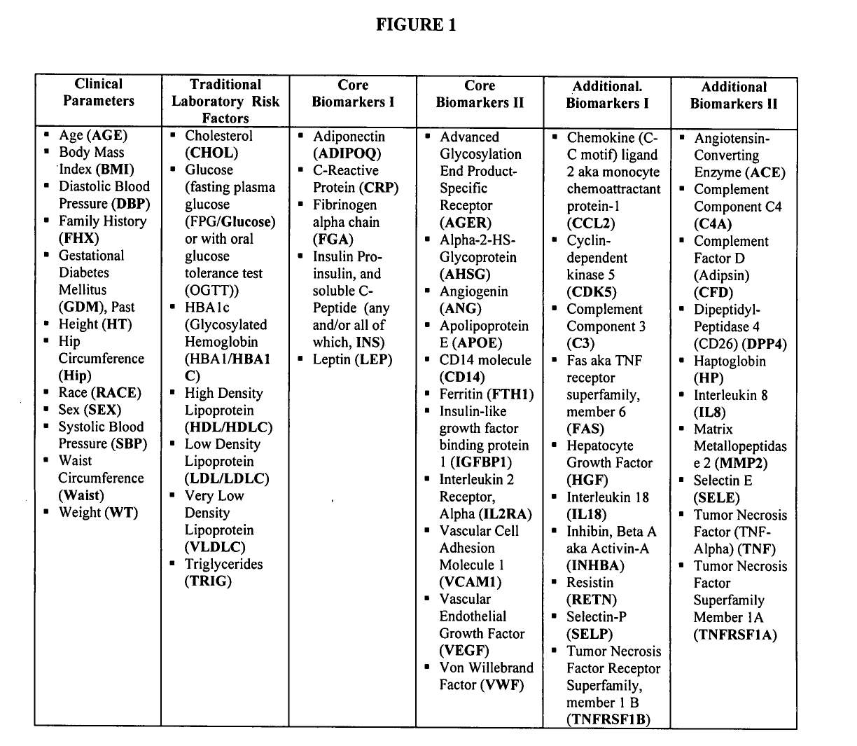

One such grouping of several classes of T2DMARKERS is presented below in Table 2 and again in FIG. 1.

TABLE 2
T2DMARKER Categories Preferred in Panel Constructions

| | Traditional | | | | |
|---|---|---|---|---|---|
| Clinical | Laboratory | Core | Core | Additional. | Additional |
| Parameters | Risk Factors | Biomarkers I | Biomarkers II | Biomarkers I | Biomarkers II |
| Age (AGE) | Cholesterol | Adiponectin | Advanced | Chemokine | Angiotensin- |
| Body Mass | (CHOL) | (ADIPOQ) | Glycosylation | (C—C motif) | Converting |
| Index | Glucose | C-Reactive | End Product- | ligand 2 aka | Enzyme |
| (BMI) | (fasting | Protein | Specific | monocyte | (ACE) |
| Diastolic | plasma | (CRP) | Receptor | chemoattract | Complement |
| Blood | glucose | Fibrinogen | (AGER) | ant protein-1 | Component |
| Pressure | (FPG/Glucose) | alpha chain | Alpha-2-HS- | (CCL2) | C4 (C4A) |
| (DBP) | or with oral | (FGA) | Glycoprotein | Cyclin- | Complement |
| Family | glucose | Insulin, | (AHSG) | dependent | Factor D |
| History | tolerance test | Pro-insulin, | Angiogenin | kinase 5 | (Adipsin) |
| (FHX) | (OGTT)) | and soluble | (ANG) | (CDK5) | (CFD) |
| Gestational | HBA1c | C-Peptide | Apolipoprotein | Complement | Dipeptidyl- |
| Diabetes | (Glycosylated | (any and/or | E (APOE) | Component | Peptidase 4 |
| Mellitus | Hemoglobin | all of | CD14 | 3 (C3) | (CD26) |
| (GDM), | (HBA1/HBA1C) | which, INS) | molecule | Fas aka TNF | (DPP4) |
| Past | High Density | Leptin | (CD14) | receptor | Haptoglobin |
| Height | Lipoprotein | (LEP) | Ferritin | superfamily, | (HP) |
| (HT) | (HDL/HDLC) | | (FTH1) | member 6 | Interleukin 8 |
| Hip | Low Density | | Insulin-like | (FAS) | (IL8) |
| Circumference | Lipoprotein | | growth factor | Hepatocyte | Matrix |
| (Hip) | (LDL/LDLC) | | binding | Growth | Metallopeptidase 2 |
| Race | Very Low | | protein 1 | Factor | (MMP2) |
| (RACE) | Density | | (IGFBP1) | (HGF) | Selectin E |
| Sex (SEX) | Lipoprotein | | Interleukin 2 | Interleukin | (SELE) |
| Systolic | (VLDLC) | | Receptor, | 18 (IL18) | Tumor |
| Blood | Triglycerides | | Alpha | Inhibin, Beta | Necrosis |
| Pressure | (TRIG) | | (IL2RA) | A aka | Factor (TNF- |
| (SBP) | | | Vascular Cell | Activin-A | Alpha) (TNF) |
| Waist | | | Adhesion | (INHBA) | Tumor |
| Circumference | | | Molecule 1 | Resistin | Necrosis |
| (Waist) | | | (VCAM1) | (RETN) | Factor |
| Weight | | | Vascular | Selectin-P | Superfamily |
| (WT) | | | Endothelial | (SELP) | Member 1A |
| | | | Growth Factor | Tumor | (TNFRSF1A) |
| | | | (VEGF) | Necrosis | |
| | | | Von | Factor | |
| | | | Willebrand | Receptor | |
| | | | Factor (VWF) | Superfamily, | |
| | | | | member 1 B | |
| | | | | (TNFRSF1B) | |

In the context of the present invention, and without limitation of the foregoing, Table 2 above may be used to construct a T2DMARKER panel comprising a series of individual T2DMARKERS. The table, derived using the above statistical and pathway informed classification techniques, is intended to assist in the construction of preferred embodiments of the invention by choosing individual T2DMARKERS from selected categories of multiple T2DMARKERS. Preferably, at least two biomarkers from one or more of the above lists of Clinical Parameters, Traditional Laboratory Risk Factors, Core Biomarkers I and II, and Additional Biomarkers I and II are selected, however, the invention also concerns selection of at least two, at least three, at least four, at least five, at least six, at least seven, at least eight, at least nine, at least ten, at least eleven, and at least twelve of these biomarkers, and larger panels up to the entire set of biomarkers listed herein. For example, at least two, at least three, at least four, at least five, at least six, at least seven, at least eight, at least nine, at least ten, at least eleven, or at least twelve biomarkers can be selected from Core Biomarkers I and II, or from Additional Biomarkers I and II.

Using the categories presented above and without intending to limit the practice of the invention, several panel selection approaches can be used independently or, when larger panels are desired, in combination in order to achieve improvements in the diagnostic accuracy of a T2DMARKER panel over the individual T2DMARKERS. A preferred approach involves first choosing one or more T2DMARKERS from the column labeled Core Biomarkers I, which represents those T2DMARKERS most frequently chosen using the various selection formula. While biomarker substitutions are possible with this approach, several biomarker selection formulas, across multiple studies and populations, have demonstrated and confirmed the importance of those T2DMARKERS listed in the Core Biomarkers I column shown above for the discrimination of subjects likely to convert to Diabetes (pre-Diabetics) from those who are not likely to do so. In general, for smaller panels, the higher performing T2DMARKER panels generally contain T2DMARKERS chosen first from the list in the Core Biomarker I column, with the highest levels of performance when several T2DMARKERS are chosen from this category. T2DMARKERS in the Core Biomarker II column can also be chosen first, and, in sufficiently large panels may also achieve high degrees of accuracy, but generally are most useful in combination with the T2DMARKERS in the Core Biomarker I column shown above.

Panels of T2DMARKERS chosen in the above fashion may also be supplemented with one or more T2DMARKERS chosen from either or both of the columns labeled Additional Biomarkers I and Additional Biomarkers II or from the columns labeled “Traditional Laboratory Risk Factors” and “Clinical Parameters.” Of the Traditional Laboratory Risk Factors, preference is given to Glucose and HBA1c. Of the Clinical Parameters, preference is given to measures of blood pressure (SBP and DBP) and of waist or hip circumference. Such Additional Biomarkers can be added to panels constructed from one or more T2DMARKERS from the Core Biomarker I and/or Core Biomarker II columns.

Finally, such Additional Biomarkers can also be used individually as initial seeds in construction of several panels together with other T2DMARKERS. The T2DMARKERS identified in the Additional Biomarkers I and Additional Biomarkers II column are identified as common substitution strategies for Core Biomarkers particularly in larger panels, and panels so constructive often still arrive at acceptable diagnostic accuracy and overall T2DMARKER panel performance. In fact, as a group, some substitutions of Core Biomarkers for Additional Biomarkers are beneficial for panels over a certain size, and can result in different models and selected sets of T2DMARKERS in the panels selected using forward versus stepwise (looking back and testing each previous T2DMARKER's individual contribution with each new T2DMARKER addition to a panel) selection formula. Multiple biomarker substitutes for individual Core Biomarkers may also be derived from substitution analysis (presenting only a constrained set of biomarkers, without the relevant Core Biomarker, to the selection formula used, and comparing the before and after panels constructed) and replacement analysis (replacing the relevant Core Biomarker with every other potential biomarker parameter, reoptimizing the formula coefficients or weights appropriately, and ranking the best replacements by a performance criteria).

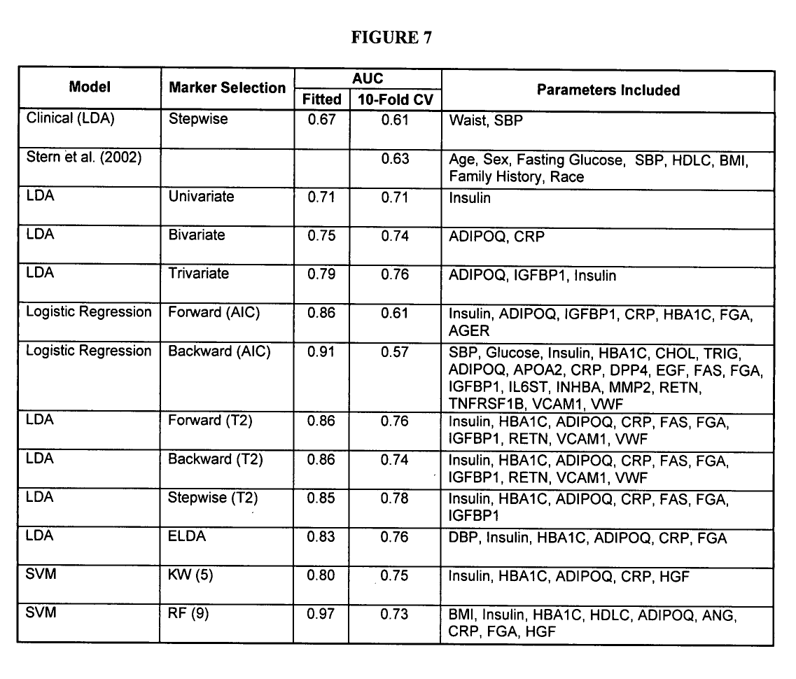

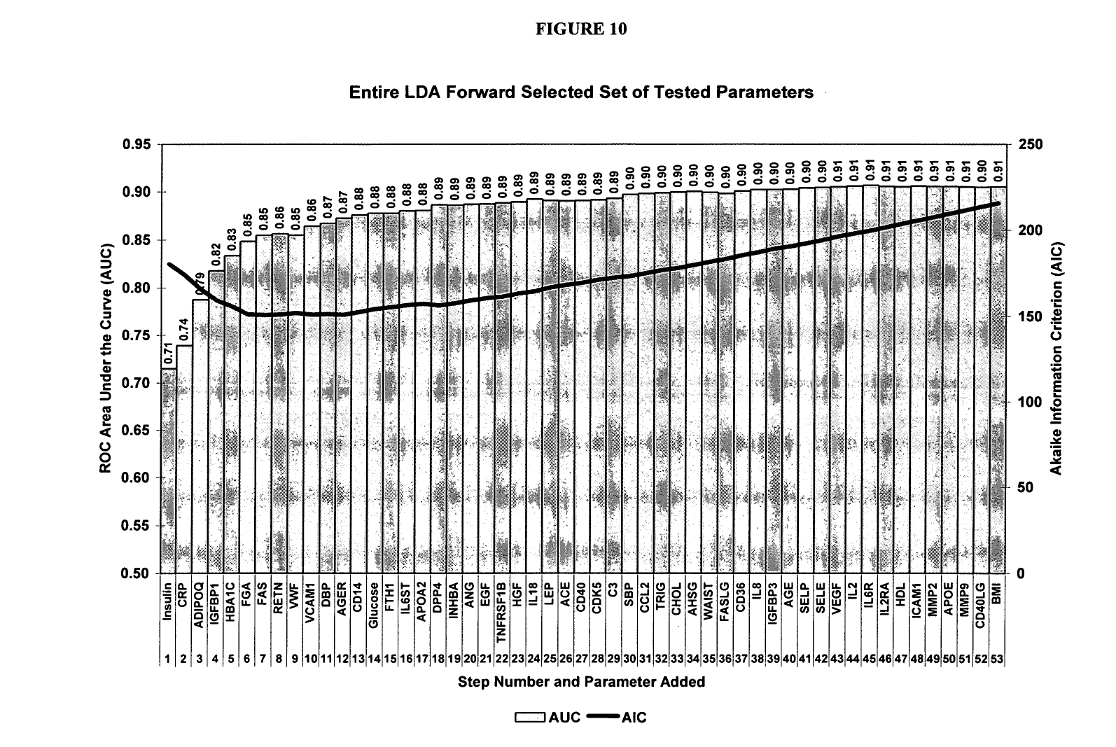

As implied above, in all such panel construction techniques, initial and subsequent Core or Additional Biomarkers, or Traditional Laboratory Risk Factors or Clinical Parameters, may also be deliberately selected from a field of many potential T2DMARKERS by T2DMARKER selection formula, including the actual performance of each derived statistical classifier algorithm itself in a training subject population, in order to maximize the improvement in performance at each incremental addition of a T2DMARKER. In this manner, many acceptably performing panels can be constructed using any number of T2DMARKERS up to the total set measured in one's individual practice of the invention (as summarized in FIG. 7, and in detail in FIGS. 10, 13, and 14 for the relevant Example populations). This technique is also of great use when the number of potential T2DMARKERS is constrained for other reasons of practicality or economics, as the order of T2DMARKER selection is demonstrated in the Examples to vary upon the total T2DMARKERS available to the formula used in selection. It is a feature of the invention that the order and identity of the specific T2DMARKERS selected under any given formula may vary based on both the starting list of potential biomarker parameters presented to the formula (the total pool from which biomarkers may be selected to form panels) as well as due to the training population characteristics and level of diversity, as shown in the Examples below.

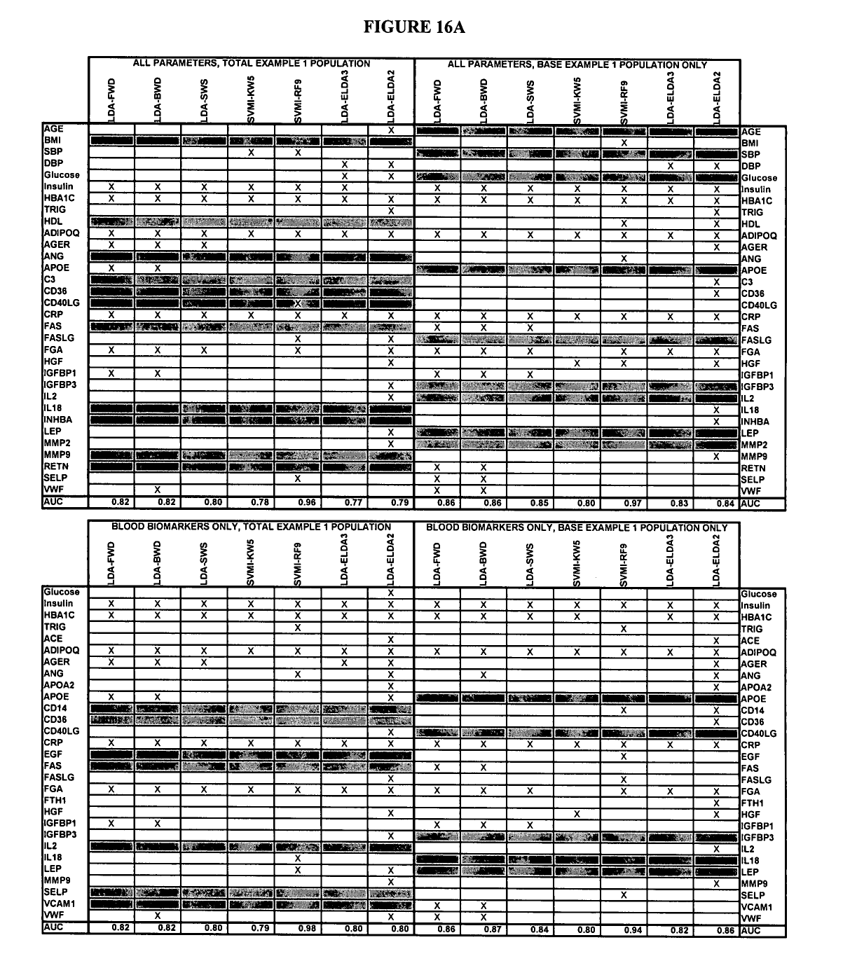

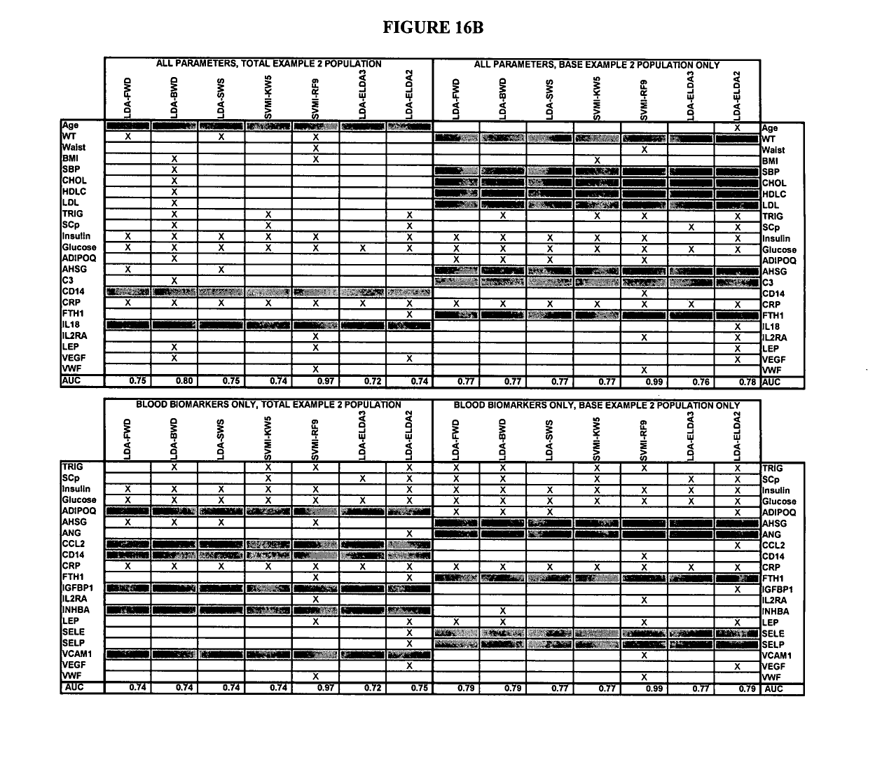

Examples of specific T2DMARKER panel construction derived using the above general techniques are also disclosed herein in the Examples, without limitation of the foregoing, our techniques of biomarker panel construction, or the applicability of alternative T2DMARKERS or biomarkers from functionally equivalent classes which are also involved in the same constituent physiological and biological pathways. Of particular note are the panels summarized in FIG. 7 for Example 1, and FIGS. 16A and 16B, which include T2DMARKERS shown in the above Tables 1 and 2 together with Traditional Laboratory Risk Factors and Clinical Parameters, and describe their AUC performance in fitted formulas within the relevant identified population and biomarker sets.

Construction of Clinical Algorithms

Any formula may be used to combine T2DMARKER results into indices useful in the practice of the invention. As indicated above, and without limitation, such indices may indicate, among the various other indications, the probability, likelihood, absolute or relative risk, time to or rate of conversion from one to another disease states, or make predictions of future biomarkers measurements of Diabetes such as Glucose or HBA1c used for Diabetes in the diagnosis of the frank disease. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from T2DMARKER results in the relevant training population. Amongst other uses, such formula may be intended to map the feature space derived from one or more T2DMARKER inputs to a set of subject classes (e.g. useful in predicting class membership of subjects as normal, pre-Diabetes, Diabetes), to derive an estimation of a probability function of risk using a Bayesian approach (e.g. the risk of Diabetes), or to estimate the class-conditional probabilities, then use Bayes' rule to produce the class probability function as in the previous case.

Prefered formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (R F, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual T2DMARKER measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art (as shown in FIG. 4 and 11, and described in Example 1, such transformation and normalization of individual biomarker concentrations may commonly be performed in the practice of the invention). Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables (much as BMI is a calculation using Height and Weight) which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves; and Vasan, R. S., 2006 regarding biomarkers of cardiovascular disease.

Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derivied using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

Modifications For Therapeutic Intervention Panels

A T2DMARKER panel can be constructed and formula derived specifically to enhance performance for use also in subjects undergoing therapeutic interventions, or a separate panel and formula may alternatively be used solely in such patient populations. An aspect of the invention is the use of sprecific known characteristics of T2DMARKERS and their changes in such subjects for such panel construction and formula derivation. Such modifications may enhance the performance of various indications noted above in Diabetes prevention, and diagnosis, therapy, monitoring, and prognosis of Diabetes and pre-Diabetes.

Several of the T2DMARKERS disclosed herein are known to those skilled in the art to vary predictably under therapeutic intervention, whether lifestyle (e.g. diet and exercise), surgical (e.g. bariatric surgery) or pharmaceutical (e.g, one of the various classes of drugs mentioned herein or known to modify common risk factors or risk of diabetes) intervention. For example, a PubMed search using the terms “Adiponectin drug,” will return over 700 references, many with respect to the changes or non-changes in the levels of adiponectin (ADIPOQ) in subjects treated with various individual Diabetes-modulating agents. Similar evidence of variance under therapeutic intervention is widely available for many of the biomarkers listed in Table 2, such as CRP, FGA, INS, LEP, among others. Certain of the biomarkers listed, most particularly the Clinical Parameters and the Traditional Laboratory Risk Factors (including such biomarkers as SBP, DBP, CHOL, HDL, and HBA1c), are traditionally used as surrogate or primary endpoint markers of efficacy for entire classes of Diabetes-modulating agents, thus most certainly changing in a statistically significant way.

Still others, including genetic biomarkers, such as those polymorphisms known in the PPARG and INSR (and generally all genetic biomarkers absent somatic mutation), are similarly known not to vary in their measurement under particular therapeutic interventions. Such variation may or may not impact the general validity of a given panel, but will often impact the index values reported, and may require different marker selection, the formula to be re-optimized or other changes to the practice of the invention. Alternative model calibrations may also be practiced in order to adjust the normally reported results under a therapeutic intervention, including the use of manual table lookups and adjustment factors.

Such properties of the individual T2DMARKERS can thus be anticipated and exploited to select, guide, and monitor therapeutic interventions. For example, specific T2DMARKERS may be added to, or subtracted from, the set under consideration in the construction of the T2DMARKER PANELS, based on whether they are known to vary, or not to vary, under therapeutic intervention. Alternatively, such T2DMARKERS may be individually normalized or formula recalibrated to adjust for such effects according to the above and other means well known to those skilled in the art.

Combination with Clinical Parameters

Any of the aforementioned Clinical Parameters may be used in the practice of the invention as a T2DMARKER input to a formula or as a pre-selection criteria defining a relevant population to be measured using a particular T2DMARKER panel and formula. As noted above, Clinical Parameters may also be useful in the biomarker normalization and pre-processing, or in T2DMARKER selection, panel construction, formula type selection and derivation, and formula result post-processing.

Measurement of T2DMARKERS

Biomarkers may be measured in using several techniques designed to achieve more predictable subject and analytical variability. On subject variability, many of the above T2DMARKERS are commonly measured in a fasting state, and most commonly in the morning, providing a reduced level of subject variability due to both food consumption and metabolism and diurnal variation. The invention hereby claims all fasting and temporal-based sampling procedures using the T2DMARKERS described herein. Pre-processing adjustments of T2DMARKER results may also be intended to reduce this effect.

The actual measurement of levels of the T2DMARKERS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, levels of T2DMARKERS can be measured using reverse-transcription-based PCR assays (RT-PCR), e.g., using primers specific for the differentially expressed sequence of genes. Levels of T2DMARKERS can also be determined at the protein level, e.g., by measuring the levels of peptides encoded by the gene products described herein, or activities thereof. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the biomarker genes according to the activity of each protein analyzed.

The T2DMARKER proteins, polypeptides, mutations, and polymorphisms thereof can be detected in any suitable manner, but is typically detected by contacting a sample from the subject with an antibody which binds the T2DMARKER protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (e.g., anti-T2DMARKER protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays include, but are not limited to oligonucleotides, immunoblotting, immunoprecipitation, immunofluorescence methods, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogeneous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Antibodies can also be useful for detecting post-translational modifications of T2DMARKER proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51).

For T2DMARKER proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant K_{M} using known algorithms, such as the Hill plot, Michaelis-Menten equation, linear regression plots such as Lineweaver-Burk analysis, and Scatchard plot.

Using sequence information provided by the database entries for the T2DMARKER sequences, expression of the T2DMARKER sequences can be detected (if present) and measured using techniques well known to one of ordinary skill in the art. For example, sequences within the sequence database entries corresponding to T2DMARKER sequences, or within the sequences disclosed herein, can be used to construct probes for detecting T2DMARKER RNA sequences in, e.g., Northern blot hybridization analyses or methods which specifically, and, preferably, quantitatively amplify specific nucleic acid sequences. As another example, the sequences can be used to construct primers for specifically amplifying the T2DMARKER sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

Alternatively, T2DMARKER protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in their entireties) In this regard, other T2DMARKER analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other T2DMARKER metabolites can be similarly detected using reagents that specifically designed or tailored to detect such metabolites.

Kits

The invention also includes a T2DMARKER-detection reagent, e.g., nucleic acids that specifically identify one or more T2DMARKER nucleic acids by having homologous nucleic acid sequences, such as oligonucleotide sequences or aptamers, complementary to a portion of the T2DMARKER nucleic acids or antibodies to proteins encoded by the T2DMARKER nucleic acids packaged together in the form of a kit. The oligonucleotides can be fragments of the T2DMARKER genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, T2DMARKER detection reagents can be immobilized on a solid matrix such as a porous strip to form at least one T2DMARKER detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of T2DMARKERS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by T2DMARKERS 1-266. In various embodiments, the expression of 2, 3, 4, 5, 6, 7, 8, 9, 10, 15, 20, 25, 40, 50, 100, 125, 150, 175, 200, 210, 220, 230, 240, 250, 260 or more of the sequences represented by T2DMARKERS 1-266 can be identified by virtue of binding to the array. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

Suitable sources for antibodies for the detection of T2DMARKERS include commercially available sources such as, for example, Abazyme, Abnova, Affinity Biologicals, AntibodyShop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, nucleic acid probes, e.g., oligonucleotides, aptamers, siRNAs, antisense oligonucleotides, against any of the T2DMARKERS in Table 1.

### EXAMPLES

Materials and Methods

Source Reagents: A large and diverse array of vendors that were used to source immunoreagents as a starting point for assay development, such as, but not limited to, Abazyme, Abnova, Affinity Biologicals, AntibodyShop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. A search for capture antibodies, detection antibodies, and analytes was performed to configure a working sandwich immunoassay. The reagents were ordered and received into inventory.

Immunoassays were developed in three steps: Prototyping, Validation, and Kit Release. Prototyping was conducted using standard ELISA formats when the two antibodies used in the assay were from different host species. Using standard conditions, anti-host secondary antibodies conjugated with horse radish peroxidase were evaluated in a standard curve. If a good standard curve was detected, the assay proceeded to the next step. Assays that had the same host antibodies went directly to the next step (e.g., mouse monoclonal sandwich assays).

Validation of working assays was performed using the Zeptosense detection platform from Singulex, Inc. (St. Louis, Mo.). The detection antibody was first conjugated to the fluorescent dye Alexa 647. The conjugations used standard NHS ester chemistry, for example, according to the manufacturer. Once the antibody was labeled, the assay was tested in a sandwich assay format using standard conditions. Each assay well was solubilized in a denaturing buffer, and the material was read on the Zeptosense platform.

Once a working Zeptosense standard curve was demonstrated, assays were typically applied to 24-96 serum samples to determine the normal distribution of the target analyte across clinical samples. The amount of serum required to measure the biomarker within the linear dynamic range of the assay was determined, and the assay proceeded to kit release. For the initial validated assays, 0.004 microliters were used per well on average.

Each component of the kit including manufacturer, catalog numbers, lot numbers, stock and working concentrations, standard curve, and serum requirements were compiled into a standard operating procedures for each biomarker assay. This kit was then released for use to test clinical samples.

### Example 1

Example 1 presents the practice of the invention in a risk matched (age, sex, BMI, among others) case-control study design. Subjects which converted to Diabetes were initially selected and risk matched based on baseline characteristic with subjects who did not convert to Diabetes, drawing from a larger longitudinal general population study. For purposes of formula discovery, subjects were selected from the larger study with the following characteristics:
Converters (C): conversion to Diabetes must have been within 5 years
Non-Converters (NC): must have had at least 8 years of follow-up with no documentation of conversion to Diabetes.

Both the “Total Population” of all such subjects and a selected “Base Population” sub-population were analyzed. The Base Population was comprised of all subjects within the Total Population who additionally met the inclusion criteria of AGE equal to or greater than 39 years and BMI equal to or greater than 25 kg/m².

Descriptive statistics summarizing each of the Example 1 study population arms are presented below in Table 3.

TABLE 3
Baseline characteristics of converters and non-converters in Cohort A

| | | Example 1 | | | |
|---|---|---|---|---|---|
| | | Total Population | | Base Population | |
| | | C | NC | C | NC |
| Variables | Levels | (n = 60) | (n = 177) | (n = 47) | (n = 120) |
| Glucose | NGT | 20 | 91 | 14 | 55 |
| tolerance status | IFG | 6 | 22 | 5 | 18 |
| baseline | IGT | 21 | 47 | 18 | 34 |
| | IFG-IGT | 13 | 17 | 10 | 13 |
| Sex | female | 28 | 84 | 22 | 60 |
| | male | 32 | 93 | 25 | 60 |
| Family HX DD | No | 8 | 21 | 6 | 14 |
| (parents and | Yes | 52 | 156 | 41 | 106 |
| sibs) | | | | | |
| Waist | Mean | 96.98 | 92.8 | 98.73 | 94.7 |
| | SD | 11.725 | 11.679 | 10.37 | 10.865 |
| | Median | 97.5 | 92.5 | 100 | 94 |
| | Min | 72 | 67.5 | 73 | 75 |
| | Max | 127 | 138 | 127 | 138 |
| | N | 60 | 177 | 47 | 120 |
| Age | Mean | 52.11 | 50.85 | 55.5 | 54.8 |
| | SD | 11.826 | 11.957 | 8.214 | 8.981 |
| | Median | 51.99 | 51.11 | 56.83 | 55.32 |
| | Min | 14.1 | 17.87 | 41.37 | 39.26 |
| | Max | 72.47 | 74.72 | 72.47 | 74.72 |
| | N | 60 | 177 | 47 | 120 |
| BMI | Mean | 28.84 | 27.76 | 29.32 | 28.71 |
| | SD | 3.889 | 4.108 | 3.557 | 3.348 |
| | Median | 28.12 | 27.17 | 28.55 | 27.72 |
| | Min | 21.98 | 19.94 | 25.14 | 25.03 |
| | Max | 43.71 | 44.55 | 43.71 | 44.55 |
| | N | 60 | 177 | 47 | 120 |
| SBP | Mean | 142.76 | 132.53 | 145.78 | 136.64 |
| | SD | 22.819 | 16.886 | 21.471 | 16.863 |
| | Median | 139.5 | 132 | 141 | 136.25 |
| | Min | 105 | 99 | 105 | 99 |
| | Max | 199 | 185 | 196 | 185 |
| | N | 60 | 177 | 47 | 120 |
| DBP | Mean | 84.78 | 81.25 | 86.47 | 83.17 |
| | SD | 10.506 | 9.653 | 10.017 | 9.422 |
| | Median | 85 | 80 | 88 | 82 |
| | Min | 62 | 56 | 67 | 60 |
| | Max | 109 | 110 | 109 | 110 |
| | N | 60 | 177 | 47 | 120 |
| CHOL | Mean | 5.9 | 5.92 | 5.94 | 6.13 |
| | SD | 1.177 | 1.245 | 1.163 | 1.253 |
| | Median | 5.67 | 5.81 | 5.71 | 6.02 |
| | Min | 4.08 | 3.39 | 4.08 | 3.77 |
| | Max | 10.04 | 12.51 | 10.04 | 12.51 |
| | N | 57 | 168 | 44 | 114 |
| HDLC | Mean | 1.28 | 1.36 | 1.22 | 1.36 |
| | SD | 0.319 | 0.31 | 0.281 | 0.33 |
| | Median | 1.25 | 1.34 | 1.16 | 1.34 |
| | Min | 0.724 | 0.776 | 0.724 | 0.776 |
| | Max | 1.959 | 2.109 | 1.893 | 2.109 |
| | N | 56 | 167 | 44 | 115 |
| TRIG | Mean | 1.7 | 1.49 | 1.75 | 1.51 |
| | SD | 1.113 | 0.88 | 0.959 | 0.79 |
| | Median | 1.58 | 1.21 | 1.62 | 1.27 |
| | Min | 0.61 | 0.508 | 0.63 | 0.587 |
| | Max | 6.57 | 6.78 | 5.56 | 3.90 |
| | N | 57 | 168 | 44 | 114 |
| Insulin | Mean | 13.09 | 8.45 | 14.04 | 8.61 |
| | SD | 8.684 | 4.553 | 9.217 | 4.393 |
| | Median | 10.5 | 7.05 | 12.92 | 7.46 |
| | Min | 2.58 | 2.72 | 2.58 | 2.90 |
| | Max | 55.50 | 27.42 | 55.50 | 24.69 |
| | N | 59 | 171 | 46 | 117 |
| Glucose | Mean | 5.94 | 5.84 | 5.94 | 5.89 |
| | SD | 0.601 | 0.572 | 0.616 | 0.569 |
| | Median | 5.94 | 5.82 | 6.05 | 5.93 |
| | Min | 4.24 | 4.63 | 4.24 | 4.63 |
| | Max | 6.89 | 6.89 | 6.89 | 6.89 |
| | N | 60 | 177 | 47 | 120 |
| Glucose 120 min | Mean | 7.92 | 6.82 | 8.05 | 6.92 |
| | SD | 2.121 | 1.541 | 2.186 | 1.437 |
| | Median | 7.95 | 6.78 | 8.14 | 7.01 |
| | Min | 4.52 | 2.60 | 4.52 | 3.62 |
| | Max | 15.82 | 10.396 | 15.82 | 10.396 |
| | N | 60 | 177 | 47 | 120 |
| HBA1C | Mean | 5.75 | 5.44 | 5.79 | 5.51 |
| | SD | 0.443 | 0.511 | 0.427 | 0.55 |
| | Median | 5.7 | 5.4 | 5.8 | 5.5 |
| | Min | 4.80 | 3.90 | 5.10 | 3.90 |
| | Max | 7.14 | 7.05 | 7.14 | 7.05 |
| | N | 53 | 138 | 41 | 93 |
| HOMA | Mean | 3.5 | 2.22 | 3.75 | 2.28 |
| | SD | 2.46 | 1.26 | 2.615 | 1.232 |
| | Median | 2.86 | 1.85 | 3.49 | 1.91 |
| | Min | 0.59 | 0.62 | 0.59 | 0.70 |
| | Max | 16.30 | 7.37 | 16.30 | 7.13 |
| | N | 59 | 171 | 46 | 117 |

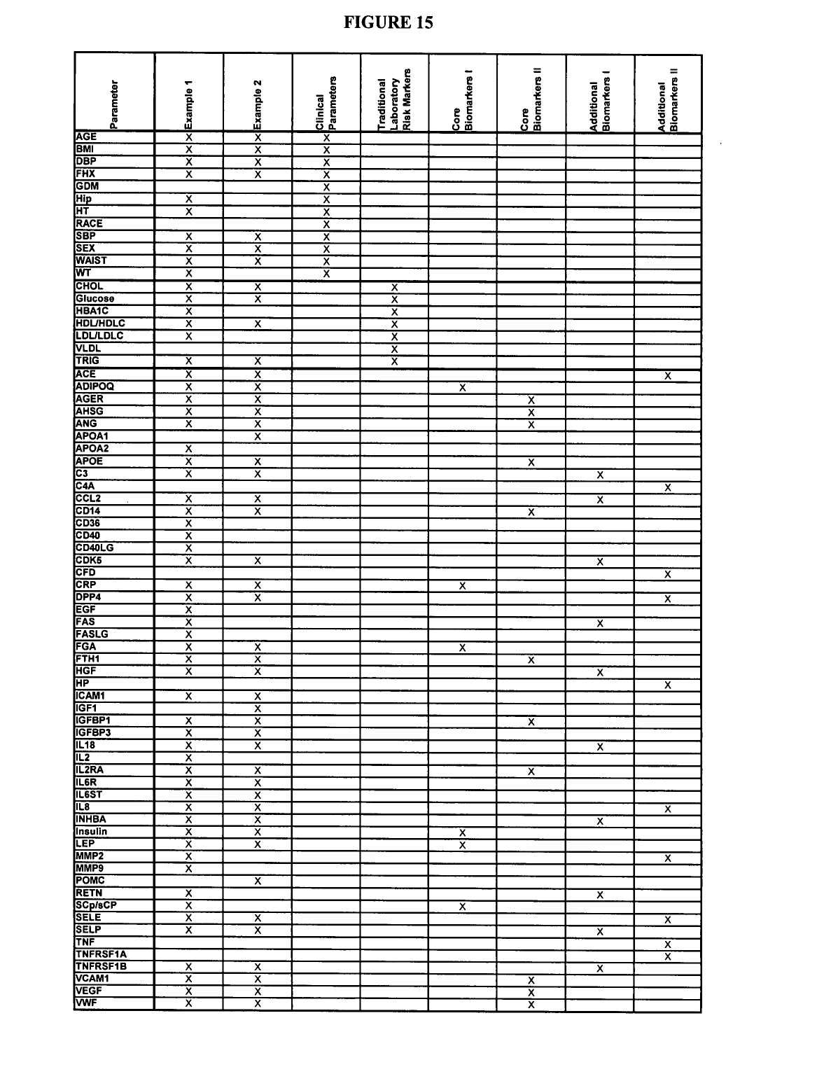

Baseline (at study entry) samples were tested. The total T2DMARKERS measured in this population are presented in FIG. 15 in the Example 1 column.

Data Analysis

Prior to statistical methods being applied, each T2DMARKER assay plate was reviewed for pass/fail criteria. Parameters taken into consideration included number of samples within range of the standard curve, serum control within the range of the standard curve, CVs of samples and dynamic range of assay.

A best fit Clinical Parameter only model was calculated in order to have a baseline to measure improvement from the incorporation of analyte-based T2DMARKERS into the potential formulas. FIG. 2 depicts a ROC curve of an LDA classification model derived only from the Clinical Parameters as measured and calculated for the Base Population of Example 1. FIG. 2 also contains the AUC as well as LOO and 10-Fold cross-validation methods. No blood-borne biomarkers were measured in this analysis.

Baseline comparison was also calculated using a common literature global Diabetes risk index encompassing selected Clinical Parameter plus selected common Traditional Risk Factors. FIG. 3 is a graphical representation of a clinical global risk assessment index according to the Stern model of Diabetes risk, measured and calculated for the Base Population of Example 1.

Prior to formula analysis, T2DMARKER parameters were transformed, according to the methodologies shown for each T2DMARKER in FIG. 4, and missing results were imputed. If the amount of missing data was greater than 1%, various imputation techniques were employed to evaluate the effect on the results, otherwise the k-nearest neighbor method (library EMV, R Project) was used using correlation as the distance metric and 6 nearest neighbors to estimate the missing values.

Excessive covariation, multicolinearity, between variables were evaluated graphically and by computing pairwise correlation coefficients. When the correlation coefficients exceeded 0.75, a strong lack of independence between biomarkers was indicated, suggesting that they should be evaluated separately. Univariate summary statistics including means, standard deviations, and odds ratios were computed using logistic regression.

FIG. 4 is a table that summarizes the results of univariate analysis of parameters variances, biomarker transformations, and biomarker mean back-transformed concentration values measured for both Converter and Non-Converter arms within Base Population of Example 1.

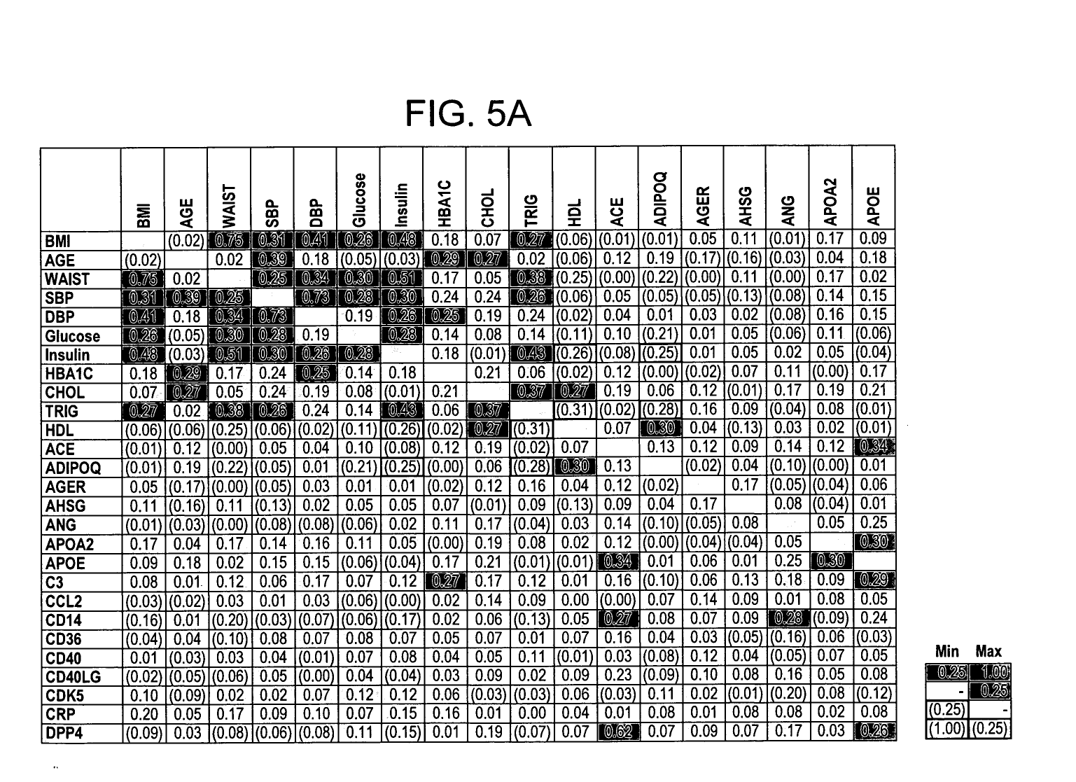

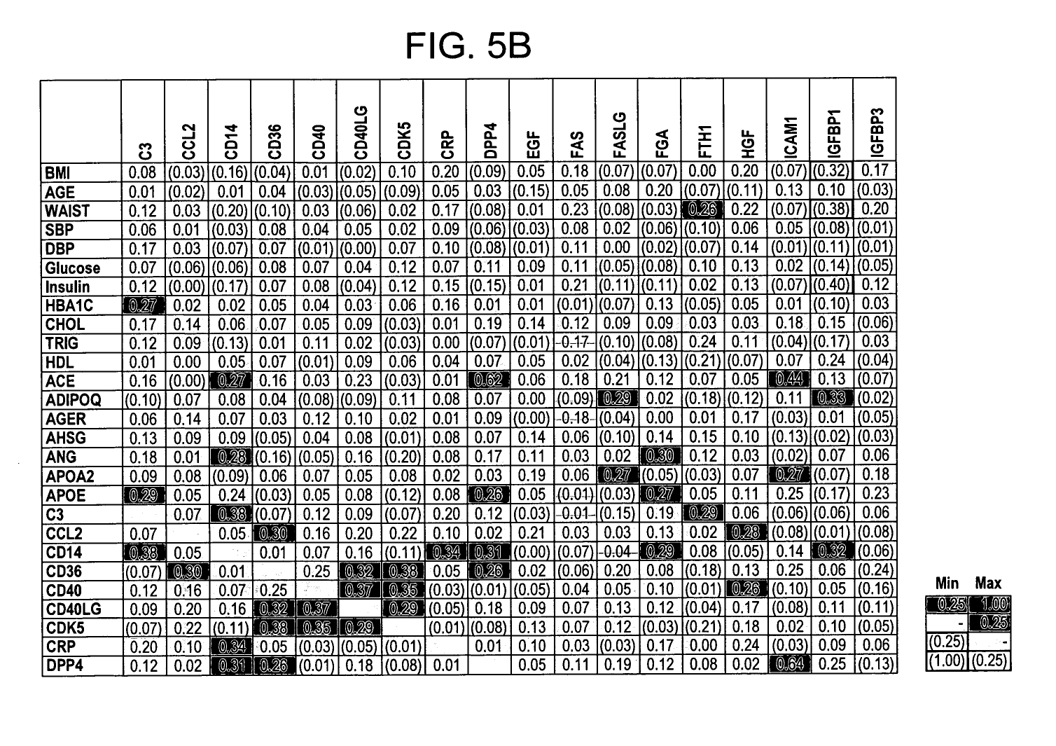

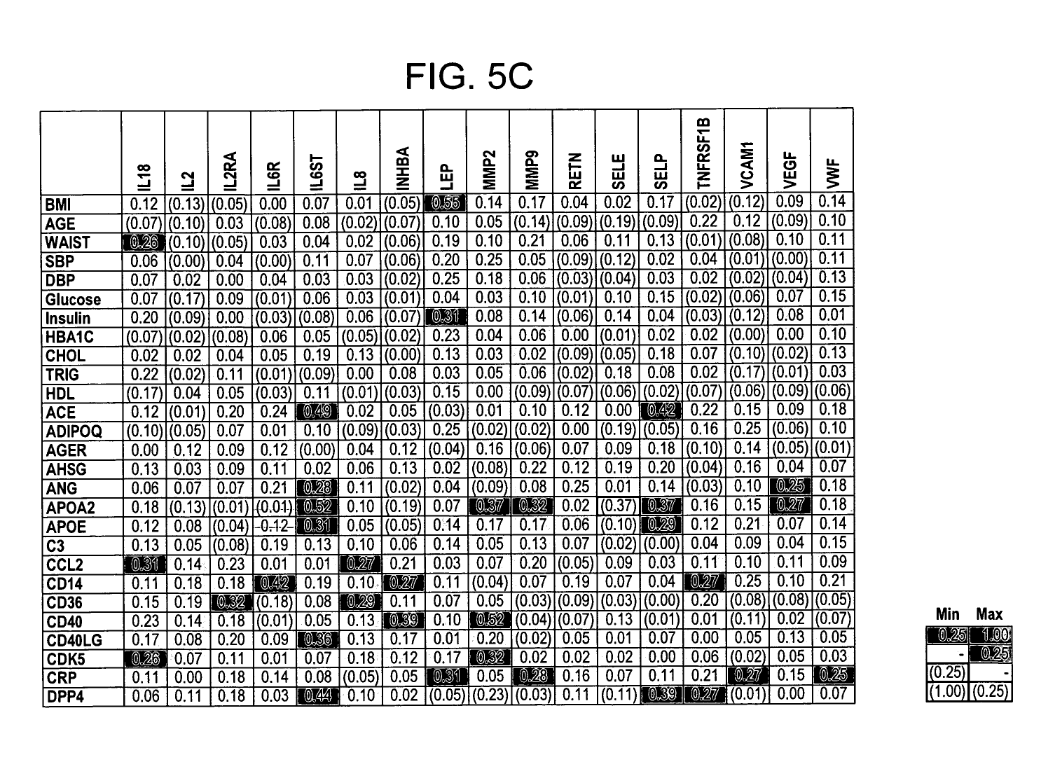

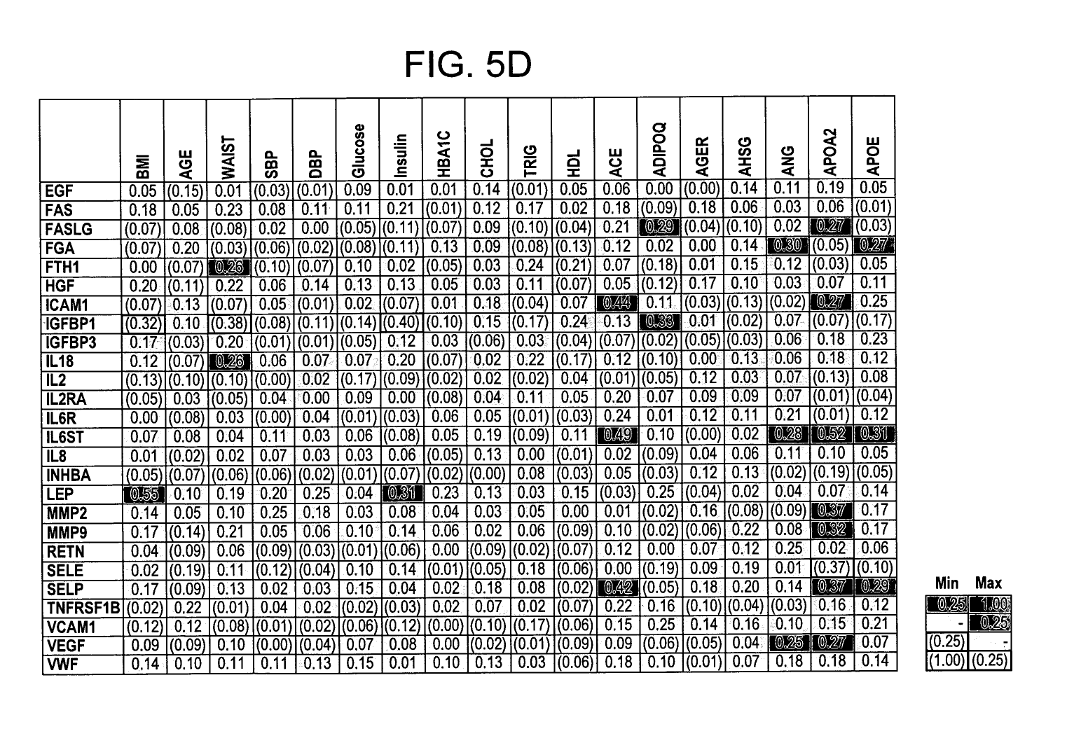

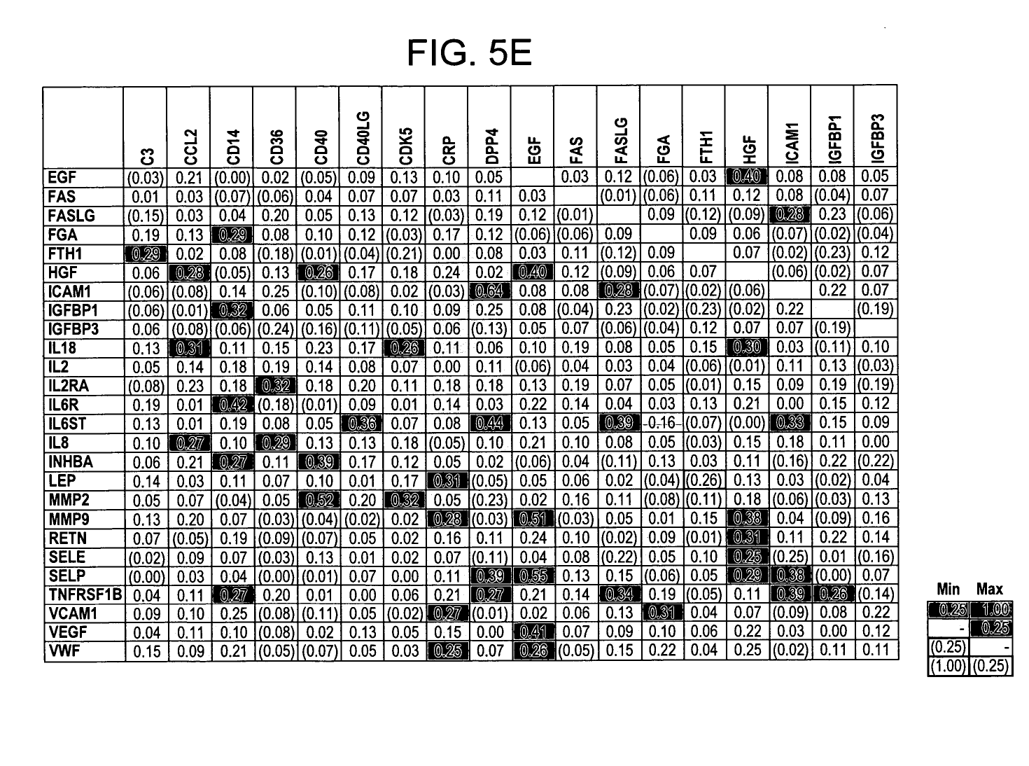

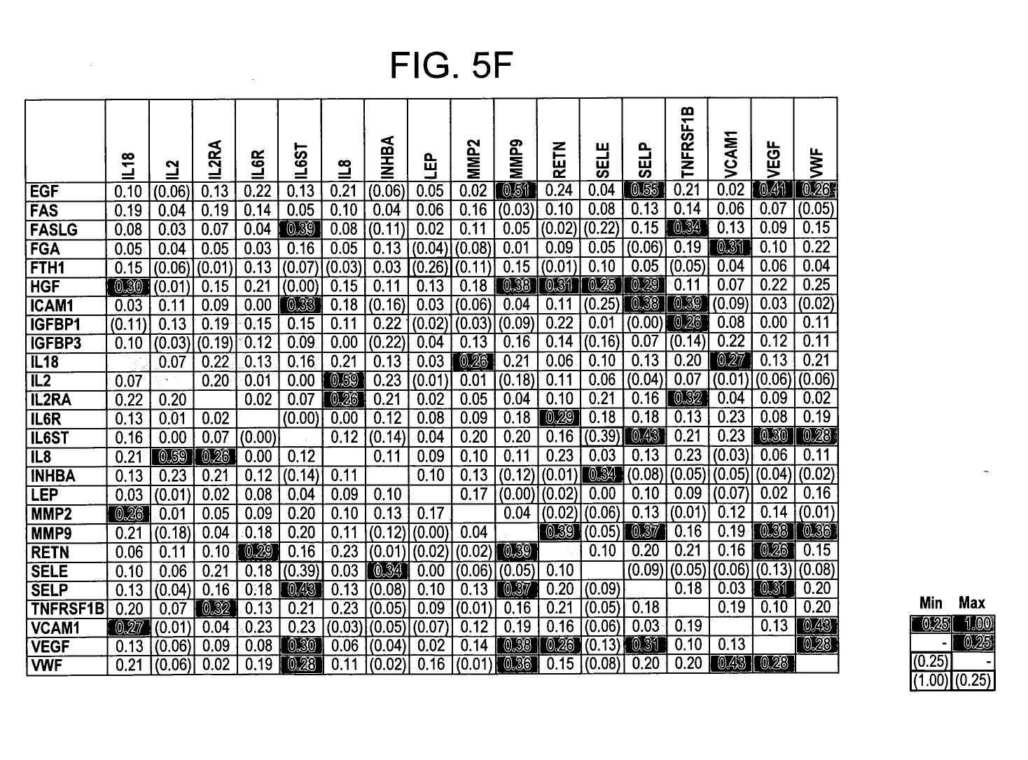

FIG. 5 presents a table summarizing a cross-correlation analysis of clinical parameters and biomarkers as disclosed herein, as measured in the Base Population of Example 1.

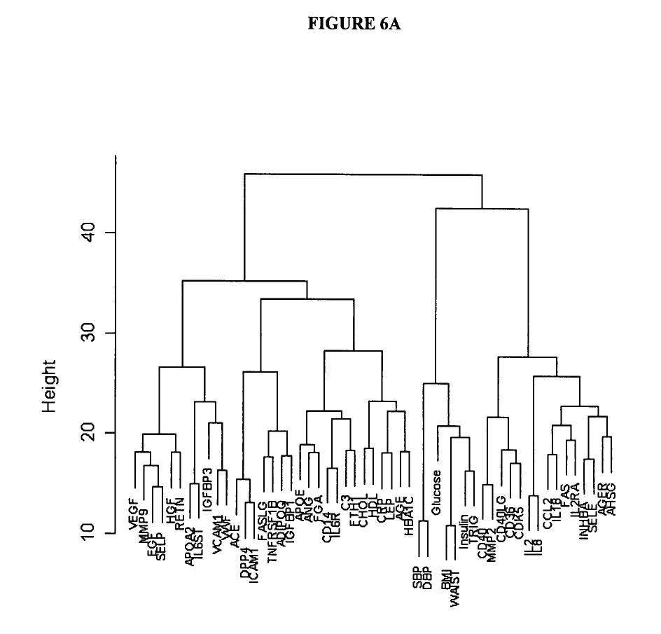

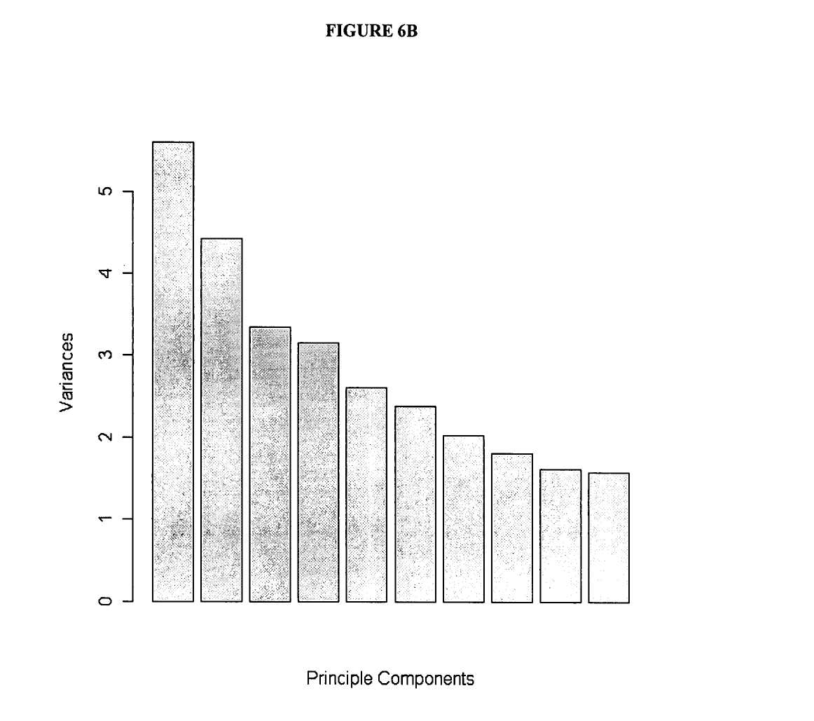

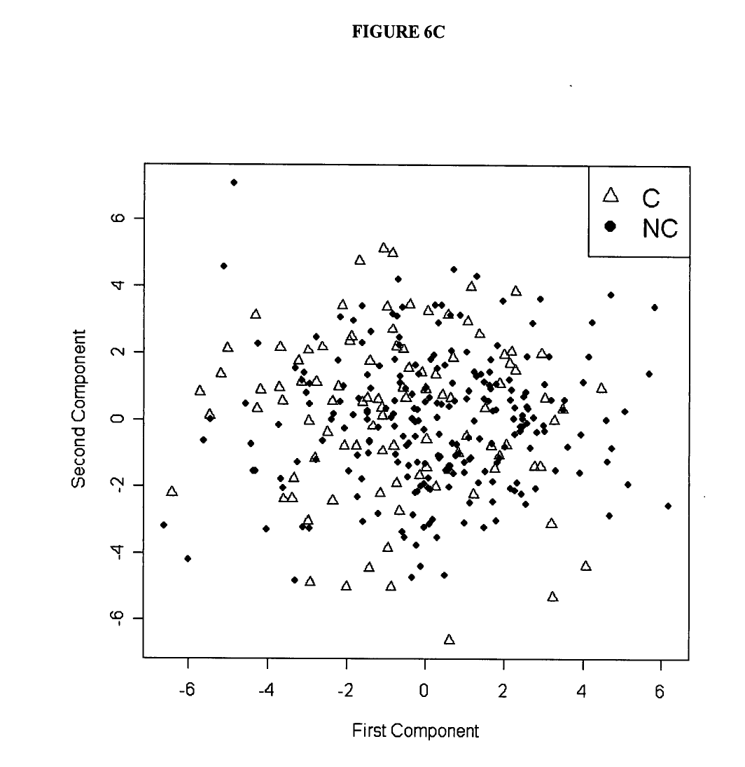

FIGS. 6A through 6C depict various graphical representations of the results of hierarchical clustering and Principal Component Analysis (PCA) of clinical parameters and biomarkers of the invention, as measured in the Base Population of Example 1.

Biomarker Selection and Model Building

Characteristics of the Base Population of Example 1 were considered in various predictive models, model types, and model parameters, and the AUC results of these formula are summarized in FIG. 7. In general, Linear Discriminant Analysis (LDA) formula maintained the most predictable performance under cross-validation.

As an example LDA model, the below coefficients represent the terms of the linear discriminant (LD) of the respective LDA models, given in the form of:
LD=coefficient1*biomarker1+coefficient2*biomarker2+coefficient3*biomarker3+

The terms “biomarker1,” “biomarker2,” “biomarker3”. . . represent the transformed values of the respective parameter as presented above in FIG. 4, with concentrations generally being log transformed, DBP being transformed using the square root function, and HBA1C value being used raw. Transformations were performed to correct the biomarkers for violations of univariate normality.

For a given subject, the posterior probability of conversion to Type 2 Diabetes Mellitus within a five year horizon under the relevant LDA is approximated by 1/(1+EXP(−1*LD). If the solution is >0.5, the subject was classified by the model as a converter.

Table 4 shows the results of ELDA and LDA SWS analysis on a selected set of T2DMARKERS and Traditional Blood Risk Factors in Cohort A Samples

TABLE 4

| | |
|---|---|
| ELDA | LDA SWS |

| | | | |
|---|---|---|---|
| DBP | −0.28145 | Insulin | −2.78863 |
| Insulin | −1.71376 | HBA1C | −0.76414 |
| HBA1C | −0.73139 | ADIPOQ | 1.818677 |
| ADIPOQ | 1.640633 | CRP | −0.83886 |
| CRP | −0.92502 | FAS | 1.041641 |
| FGA | 0.955317 | FGA | 0.827067 |
| | | IGFBP1 | −1.2481 |

Model Validation

To validate both the biomarker selection process and the underlying predictive algorithm, extensive cross-validation incorporating both feature selection and algorithm estimation was used. Two common cross-validation schemes to determine model performance were used. A leave-one-out CV is known to produce nearly unbiased prediction error estimates, but the estimate is often criticized to be highly variable. A 10-fold cross-validation, on the other hand, reduces the variability, but can introduce bias in the error estimates (Braga-Neto and Dougherty, 2004). To reduce the bias in this estimate the 10-fold cross validation was repeated 10 times such that the training samples were randomly divided 100 times into training groups consisting of 90% of the samples and test groups consisting of the remaining 10% of the samples. Such repeated 10-fold CV estimator has been recommended as an overall error estimator of choice in terms of reduced variance (Kohavi, 1995). The model performance characteristics were then averaged over all 10 of the cross validations.

Biomarker importance was estimated by ranking the features by their appearance frequencies in all the CV steps, because biomarker selection was carried out within the CV loops. Model quality was evaluated based on the model with the largest area under the ROC curve as well as sensitivity and specificity at the limit of the region of the ROC curve with the greatest area (i.e. the inflection point of the sensitivity plots).

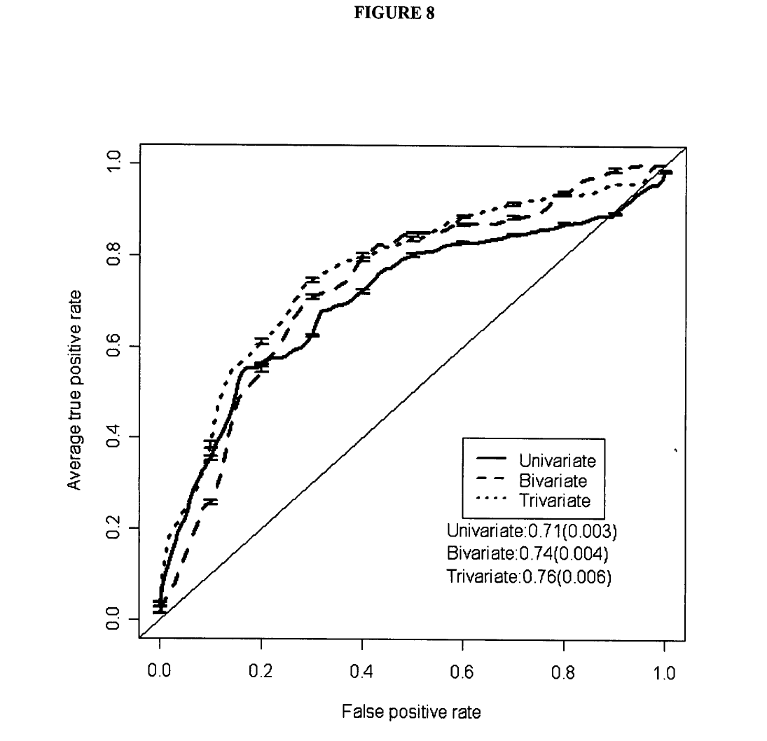

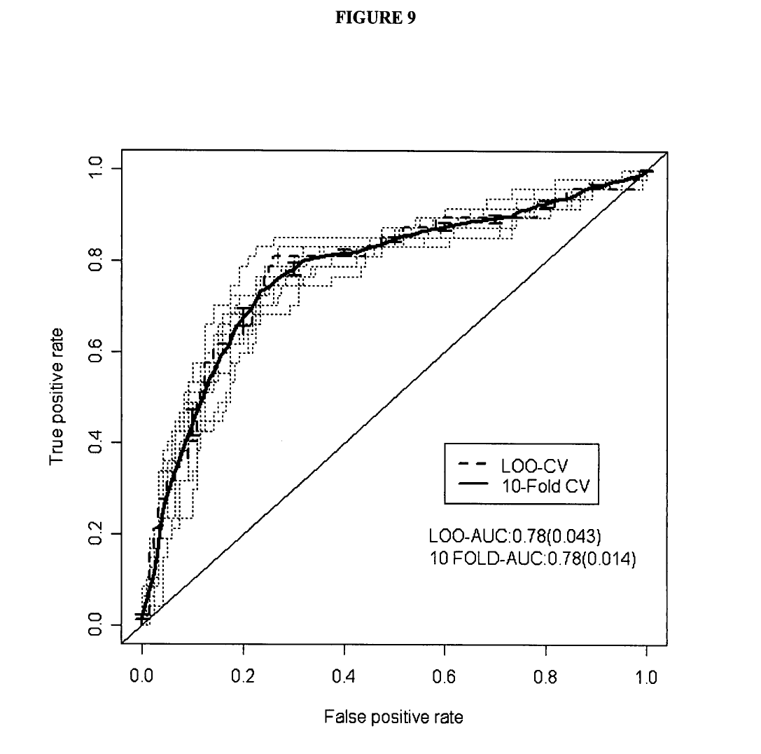

FIG. 8 is a graph showing the ROC curves for the leading univariate, bivariate, and trivariate LDA models by AUC, as measured and calculated in the Base Population of Example 1, whereas FIG. 9 graphically shows ROC curves for the LDA stepwise selection model, also as measured and calculated in the Base Population of Example 1. The entire LDA forward-selected set of all tested parameters with model AUC and Akaike Information Criterion (AIC) statistics at each biomarker addition step is shown in the graph of FIG. 10, as measured and calculated in the Base Population of Example 1.

### Example 2

Example 2 demonstrates the practice of the invention in a separate general longitudinal population-based study, with a comparably selected Base sub-population and a frank Diabetes sub-analysis.

As in Example 1, for purposes of model discovery, subjects were selected from the sample sets with the following characteristics:
Converters (C): conversion to Diabetes must have been within 5 years
Non-Converters (NC): must have had at least 8 years of follow-up with no documentation of Diabetes.

As in Example 1, both the “Total Population” of all such subjects and a selected “Base Population” sub-population were analyzed. The Base Population was comprised of all subjects within the Total Population who additionally met the inclusion criteria of AGE equal to or greater than 39 years and BMI equal to or greater than 25 kg/m².

Descriptive statistics summarizing each of the Example 2 study population arms are presented below in Table 5.

TABLE 5
Baseline Characteristics of Cohort B and Subsets

| | | Example 2 | | | | |
|---|---|---|---|---|---|---|
| | | Total Population | | Base Population | | |
| | | | NC | C | NC | Diabetic |
| Variables | Levels | C (n = 100) | (n = 236) | (n = 83) | (n = 236) | (n = 48) |
| HeartThrombosis | No | 95 | 225 | 78 | 225 | 45 |
| | Yes | 0 | 1 | 0 | 1 | 1 |
| PhysicalActivity | Active | 12 | 32 | 12 | 32 | 4 |
| | Athelete | 0 | 3 | 0 | 3 | 1 |
| | Sit | 26 | 50 | 24 | 50 | 21 |
| | Walk | 60 | 146 | 45 | 146 | 21 |
| Familial History | No | 94 | 211 | 78 | 211 | 45 |
| of CVD | Yes | 6 | 25 | 5 | 25 | 3 |
| Glucose tolerance | NGT | 21 | 163 | 14 | 163 | 0 |
| status baseline | IFG | 18 | 39 | 15 | 39 | 0 |
| | IGT | 59 | 27 | 52 | 27 | 0 |
| | SDM | 0 | 0 | 0 | 0 | 27 |
| | KDM | 0 | 0 | 0 | 0 | 21 |
| Diet | average | 57 | 160 | 46 | head | 27 |
| | healthy | 13 | 34 | 13 | 34 | 9 |
| | unhealthy | 23 | 31 | 18 | 31 | 9 |
| Sex | female | 39 | 91 | 31 | 91 | 19 |
| | male | 61 | 145 | 52 | 145 | 29 |
| Family HX DD | No | 71 | 182 | 57 | 182 | 32 |
| (parents and sibs) | Yes | 29 | 54 | 26 | 54 | 16 |
| Family HX DB | No | 97 | 236 | 81 | 236 | 47 |
| (children) | Yes | 3 | 0 | 2 | 0 | 1 |
| High Risk | No | 9 | 79 | 5 | 79 | 0 |
| | Yes | 91 | 157 | 78 | 157 | 48 |
| Smoking | Not Offered | 59 | 90 | 53 | 90 | 39 |
| Intervention | Declined | 21 | 43 | 16 | 43 | 6 |
| | Accepted | 11 | 24 | 9 | 24 | 3 |
| Diet and Exercise | Not Offered | 14 | 62 | 9 | 62 | 12 |
| Intervention | Declined | 22 | 36 | 19 | 36 | 11 |
| | Accepted | 55 | 59 | 50 | 59 | 25 |
| Height | Mean | 172.4 | 172.97 | 172.43 | 172.97 | 170.85 |
| | SD | 9.112 | 9.486 | 9.445 | 9.486 | 10.664 |
| | Median | 172 | 173 | 172 | 173 | 170.5 |
| | Min | 148 | 151 | 148 | 151 | 149 |
| | Max | 192 | 195 | 192 | 195 | 194 |
| | N | 100 | 236 | 83 | 236 | 48 |
| Weight | Mean | 87.44 | 86.35 | 90.61 | 86.35 | 90.98 |
| | SD | 16.398 | 14.457 | 14.968 | 14.457 | 18.396 |
| | Median | 84.5 | 84.45 | 88 | 84.45 | 86.3 |
| | Min | 49.8 | 57 | 67.2 | 57 | 64.3 |
| | Max | 126 | 183 | 126 | 183 | 141.2 |
| | N | 100 | 236 | 83 | 236 | 48 |
| Waist | Mean | 96.05 | 93.39 | 98.49 | 93.39 | 101.31 |
| | SD | 12.567 | 11.05 | 11.651 | 11.05 | 13.246 |
| | Median | 94.5 | 93 | 96 | 93 | 99 |
| | Min | 66 | 68 | 72 | 68 | 79 |
| | Max | 125 | 165 | 125 | 165 | 136 |
| | N | 100 | 235 | 83 | 235 | 48 |
| Hip | Mean | 105.34 | 105.37 | 106.72 | 105.37 | 108.02 |
| | SD | 9.47 | 9.774 | 9.021 | 9.774 | 11.412 |
| | Median | 105.5 | 104 | 107 | 104 | 105.5 |
| | Min | 81 | 88 | 81 | 88 | 91 |
| | Max | 135 | 165 | 135 | 165 | 151 |
| | N | 100 | 235 | 83 | 235 | 48 |
| Age | Mean | 49.6 | 48.81 | 50.07 | 48.81 | 51.26 |
| | SD | 6.786 | 6.325 | 6.325 | 6.325 | 6.426 |
| | Median | 50 | 49.8 | 50 | 49.8 | 50.15 |
| | Min | 34.7 | 39.7 | 39.8 | 39.7 | 39.8 |
| | Max | 60.5 | 60.3 | 60.5 | 60.3 | 60.8 |
| | N | 100 | 236 | 83 | 236 | 48 |
| BMI | Mean | 29.36 | 28.82 | 30.42 | 28.82 | 31.13 |
| | SD | 4.656 | 4.115 | 4.051 | 4.115 | 5.472 |
| | Median | 28.7 | 27.65 | 29.7 | 27.65 | 29.8 |
| | Min | 18.7 | 25 | 25 | 25 | 25 |
| | Max | 45.2 | 55.7 | 45.2 | 55.7 | 48.9 |
| | N | 100 | 236 | 83 | 236 | 48 |
| Units of alcohol | Mean | 12.61 | 13.68 | 12.3 | 13.68 | 15.55 |
| intake per week | SD | 13.561 | 28.03 | 13.419 | 28.03 | 22.115 |
| | Median | 6 | 8 | 6 | 8 | 6.5 |
| | Min | 0 | 0 | 0 | 0 | 0 |
| | Max | 59 | 330 | 59 | 330 | 102 |
| | N | 95 | 219 | 79 | 219 | 44 |
| SBP | Mean | 138.07 | 133.91 | 139.18 | 133.91 | 144.15 |
| | SD | 18.265 | 18.508 | 15.798 | 18.508 | 23.448 |
| | Median | 140 | 130 | 140 | 130 | 140 |
| | Min | 104 | 100 | 110 | 100 | 100 |
| | Max | 195 | 198 | 180 | 198 | 212 |
| | N | 100 | 236 | 83 | 236 | 48 |
| DBP | Mean | 87.28 | 84.91 | 87.61 | 84.91 | 87.1 |
| | SD | 12.874 | 11.708 | 12.151 | 11.708 | 10.446 |
| | Median | 85 | 85 | 85 | 85 | 87 |
| | Min | 58 | 60 | 66 | 60 | 60 |
| | Max | 140 | 128 | 140 | 128 | 110 |
| | N | 100 | 236 | 83 | 236 | 48 |
| CHOL | Mean | 5.92 | 5.81 | 5.95 | 5.81 | 5.85 |
| | SD | 1.092 | 1.033 | 1.033 | 1.033 | 1.015 |
| | Median | 5.8 | 5.7 | 5.8 | 5.7 | 5.9 |
| | Min | 3.4 | 3.5 | 3.6 | 3.5 | 4.1 |
| | Max | 9.2 | 9 | 8.5 | 9 | 7.7 |
| | N | 100 | 236 | 83 | 236 | 48 |
| HDLC | Mean | 1.29 | 1.35 | 1.26 | 1.35 | 1.25 |
| | SD | 0.352 | 0.388 | 0.343 | 0.388 | 0.35 |
| | Median | 1.23 | 1.29 | 1.21 | 1.29 | 1.21 |
| | Min | 0.66 | 0.6 | 0.66 | 0.6 | 0.74 |
| | Max | 2.19 | 3.37 | 2.19 | 3.37 | 2.6 |
| | N | 100 | 236 | 83 | 236 | 48 |
| LDL | Mean | 3.8 | 3.75 | 3.83 | 3.75 | 3.62 |
| | SD | 0.992 | 0.912 | 0.952 | 0.912 | 0.843 |
| | Median | 3.7 | 3.7 | 3.72 | 3.7 | 3.6 |
| | Min | 1.61 | 1.2 | 2.1 | 1.2 | 1.6 |
| | Max | 6.62 | 6.86 | 6.62 | 6.86 | 5.4 |
| | N | 97 | 232 | 80 | 232 | 45 |
| TRIG | Mean | 1.92 | 1.6 | 2 | 1.6 | 2.2 |
| | SD | 1.107 | 1.454 | 1.143 | 1.454 | 1.444 |
| | Median | 1.6 | 1.3 | 1.6 | 1.3 | 1.9 |
| | Min | 0.5 | 0.4 | 0.6 | 0.4 | 0.6 |
| | Max | 5.6 | 15.2 | 5.6 | 15.2 | 7 |
| | N | 100 | 236 | 83 | 236 | 48 |
| SCp0 | Mean | 652.08 | 595.81 | 670.23 | 595.81 | 706.33 |
| | SD | 197.944 | 177.582 | 197.384 | 177.582 | 195.637 |
| | Median | 659.5 | 564 | 706.5 | 564 | 727 |
| | Min | 280 | 273 | 280 | 273 | 10 |
| | Max | 972 | 988 | 972 | 988 | 996 |
| | N | 72 | 209 | 56 | 209 | 33 |
| Insulin | Mean | 63.14 | 45.85 | 67.24 | 45.85 | 71.26 |
| | SD | 39.01 | 28.065 | 40.203 | 28.065 | 38.414 |
| | Median | 53.5 | 37 | 57 | 37 | 62 |
| | Min | 12 | 10 | 12 | 10 | 26 |
| | Max | 210 | 164 | 210 | 164 | 217 |
| | N | 100 | 236 | 83 | 236 | 47 |
| Ins120 | Mean | 382.89 | 213.13 | 401.88 | 213.13 | 464.34 |
| | SD | 231.912 | 157.625 | 227.478 | 157.625 | 295.239 |
| | Median | 323.5 | 181 | 351.5 | 181 | 441 |
| | Min | 55 | 11 | 55 | 11 | 53 |
| | Max | 958 | 913 | 958 | 913 | 990 |
| | N | 90 | 224 | 74 | 224 | 32 |
| Glucose | Mean | 5.95 | 5.61 | 6 | 5.61 | 8.91 |
| | SD | 0.55 | 0.504 | 0.528 | 0.504 | 3.843 |
| | Median | 6 | 5.6 | 6 | 5.6 | 7.3 |
| | Min | 4.7 | 4.1 | 4.7 | 4.1 | 4.9 |
| | Max | 6.8 | 6.9 | 6.8 | 6.9 | 21 |
| | N | 100 | 236 | 83 | 236 | 48 |
| Glucose 120 min | Mean | 8.07 | 6.08 | 8.22 | 6.08 | 12.5 |
| | SD | 1.876 | 1.543 | 1.791 | 1.543 | 4.349 |
| | Median | 8.5 | 6 | 8.6 | 6 | 12.5 |
| | Min | 4 | 2.4 | 4 | 2.4 | 4.2 |
| | Max | 11 | 10.7 | 11 | 10.7 | 25.6 |
| | N | 98 | 229 | 81 | 229 | 36 |

T2DMARKER biomarkers were run on baseline samples in the same manner as described for the samples derived from Example 2.

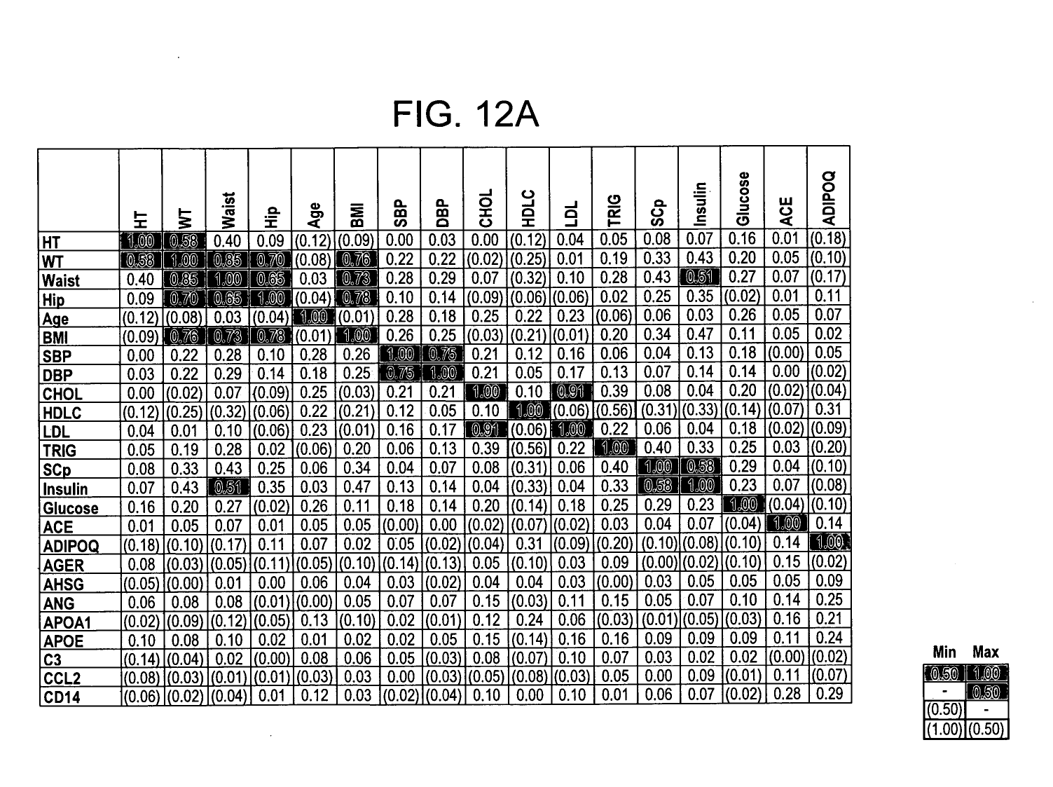

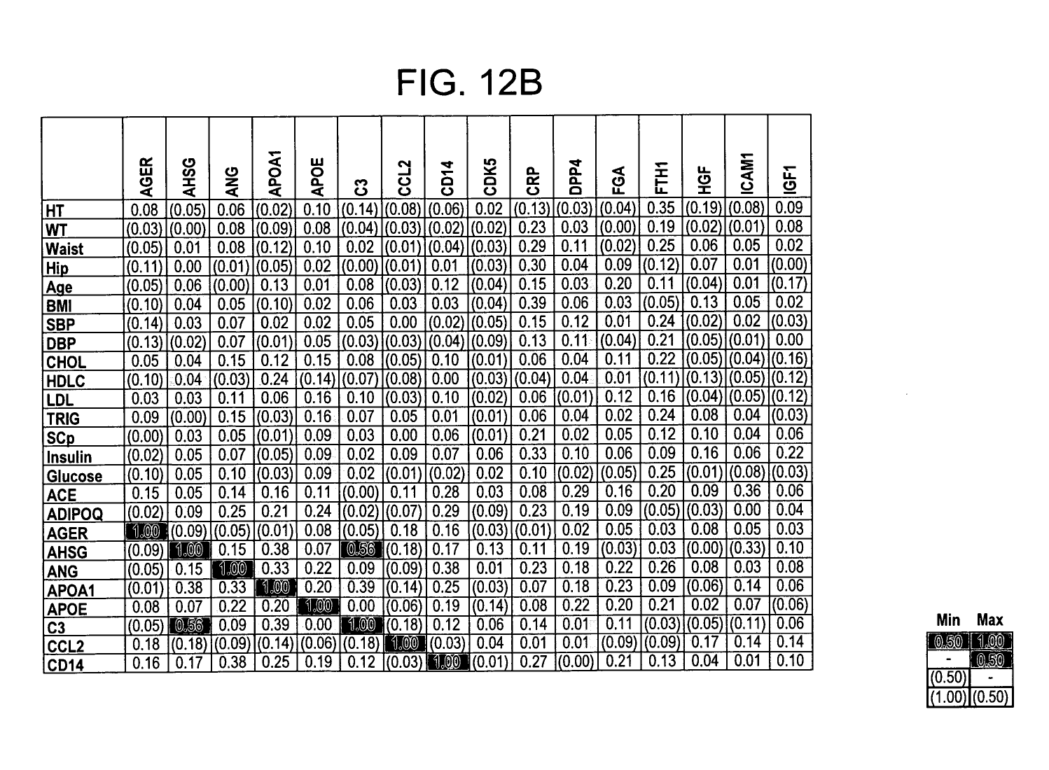

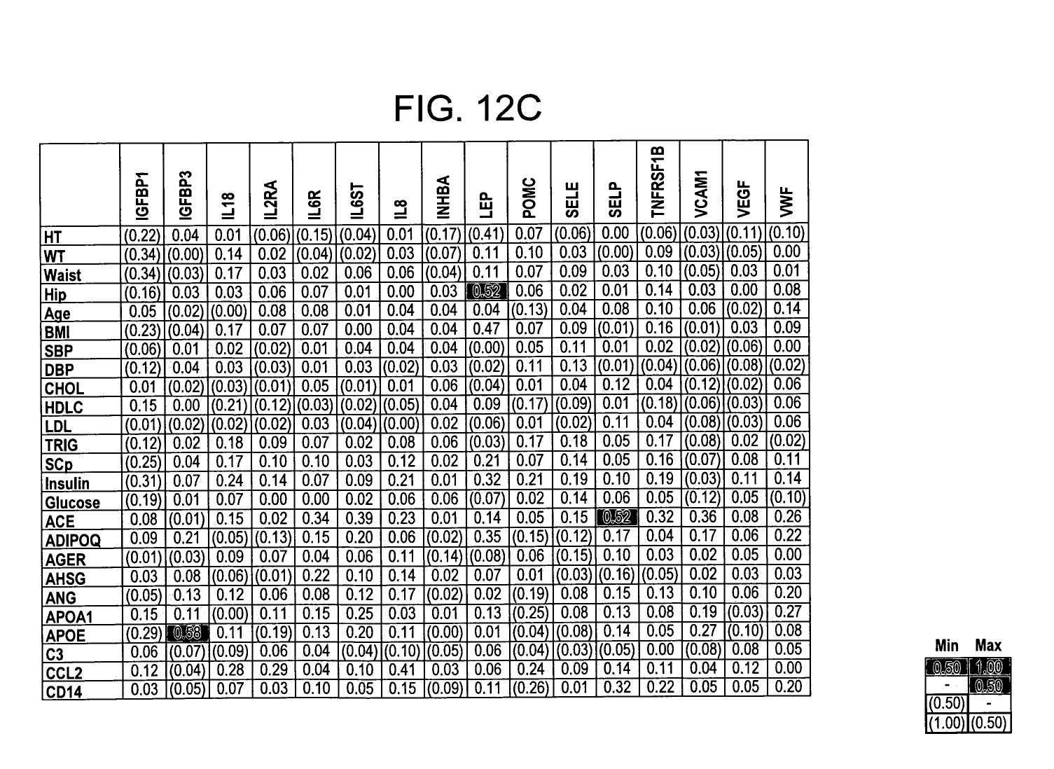

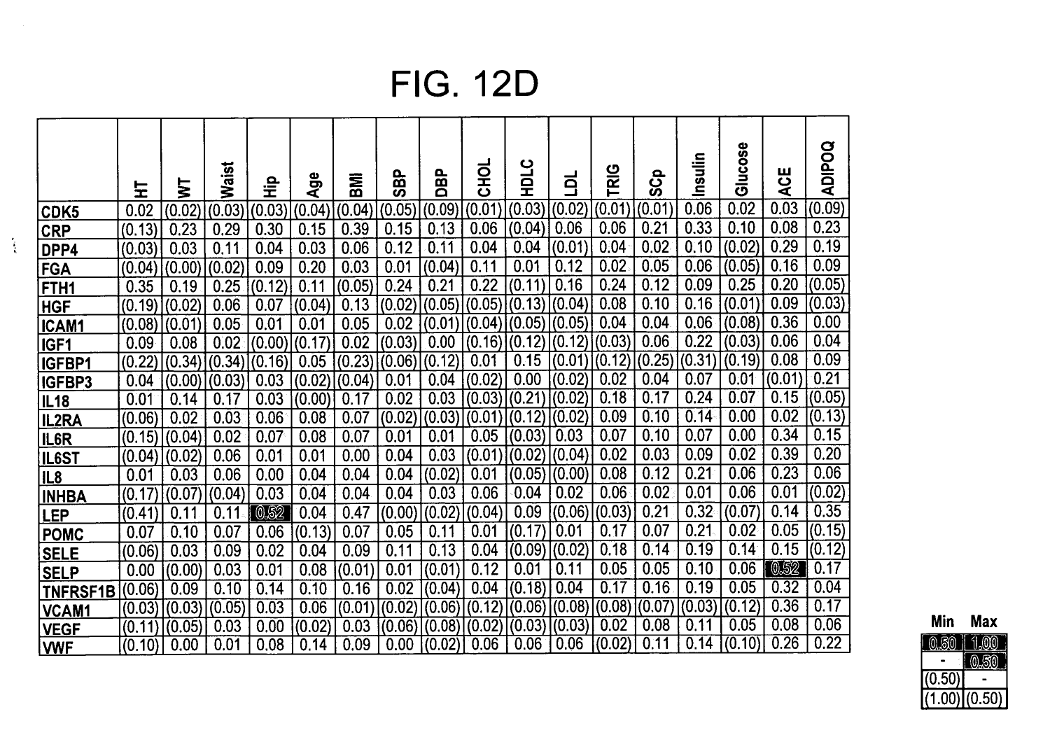

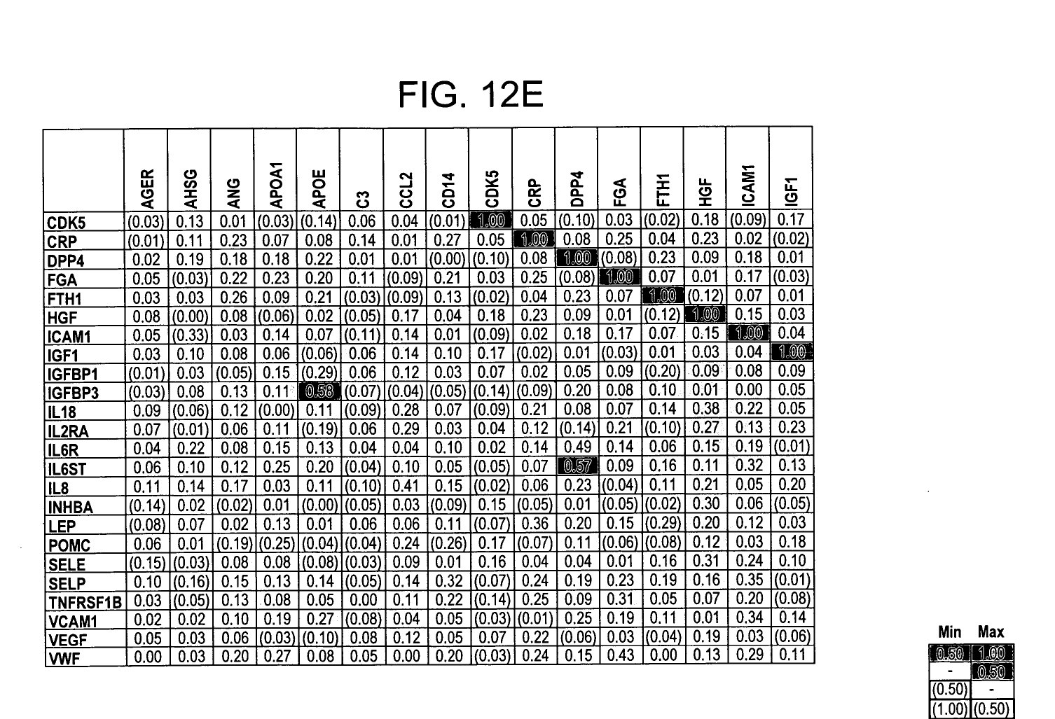

FIG. 11 shows tables that summarize univariate ANOVA analyses of parameter variances, including biomarker transformation and biomarker mean back-transformed concentration values across non-converters, converters, and diabetic populations, as measured and calculated at baseline in the Total Population of Example 2. Cross-correlation of clinical parameters and selected biomarkers are shown in FIG. 12, which was measured in the Total Populations of Example 2.

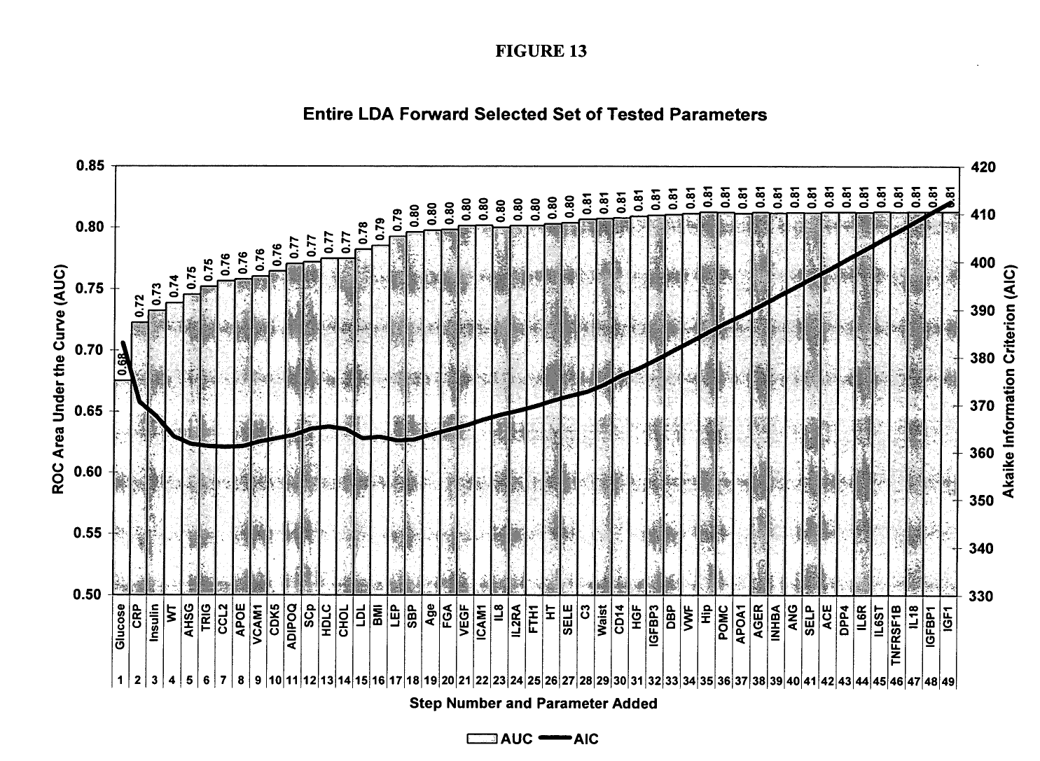

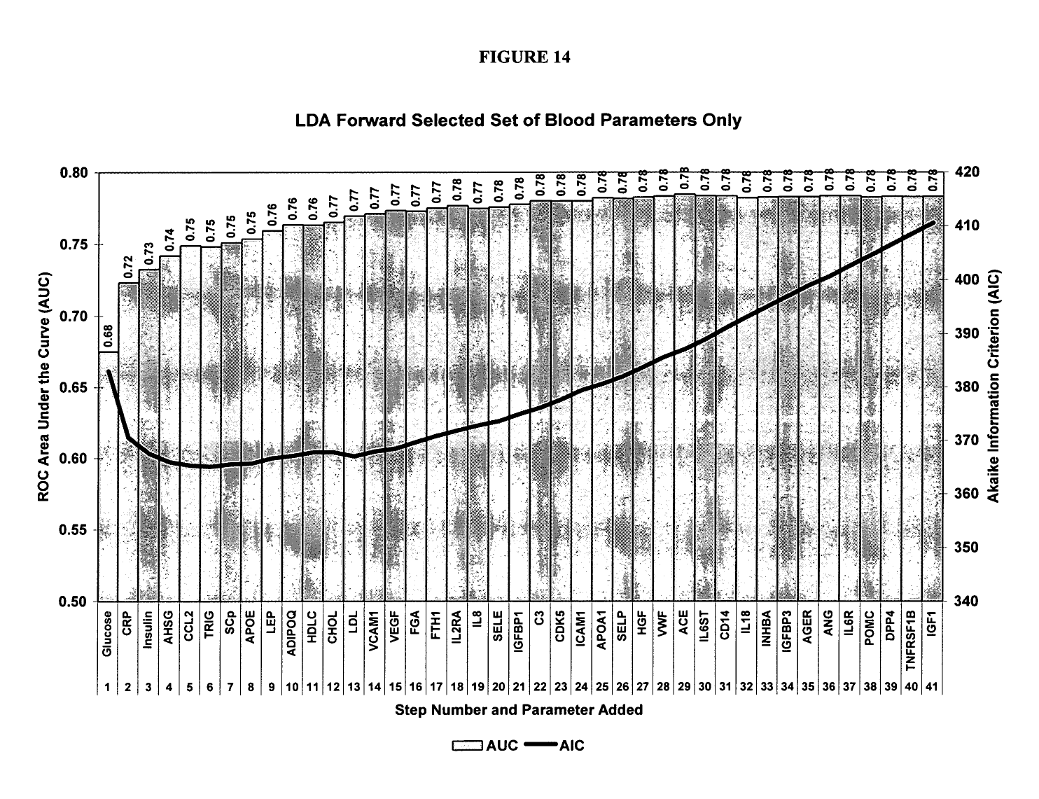

FIG. 13 is a graphical representation of the entire LDA forward-selected set of tested parameters with model AUC and AIC statistics at each biomarker addition step, as measured and calculated in the Total Population of Example 2, while FIG. 14 graphically shows an LDA forward-selected set of blood-borne biomarkers (excluding clinical parameters) alone with model characteristics at each biomarker addition step as described herein in the same population.

### Example 3

Example 3 is a study of the differences and similiarities between the results obtained in the two previous Examples.

FIG. 15 is a tabular representation of all parameters tested in Example 1 and Example 2, according to the T2DMARKER biomarker categories disclosed herein.

Tables summarizing T2DMARKER biomarker selection under various scenarios of classification model types and base and total populations of Examples 1 and 2 are shown in FIGS. 16A and 16B, respectively.

FIG. 17 further summarizes the complete enumeration of fitted LDA models for all potential univariate, bivariate, and trivariate combinations as measured and calculated for both Total and Base Populations of Examples 1 and 2, and encompassing all 53 and 49 T2DMARKER parameters recorded, respectively, for each study as potential model parameters. A graphical representation of the data presented in FIG. 17 is shown in FIG. 18, which shows the number and percentage of the total univariate, bivariate, and trivariate models that meet various AUC hurdles using the Total Population of Example 1.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Aalto, K., L. Korhonen, et al. (2002). “Nerve growth factor in serum of children with systemic lupus erythematosus is correlated with disease activity.” Cytokine 20(3): 136-9.
Abate, N., M. Chandalia, et al. (2005). “ENPP1/PC-1 K121Q polymorphism and genetic susceptibility to type 2 Diabetes.” Diabetes 54(4): 1207-13.
Adam, T. C., J. Jocken, et al. (2005). “Decreased glucagon-like peptide 1 release after weight loss in overweight/obese subjects.” Obes Res 13(4): 710-6.
Aguilar-Bryan, L., C. G. Nichols, et al. (1995). “Cloning of the beta cell high-affinity sulfonylurea receptor: a regulator of insulin secretion.” Science 268(5209): 423-6.
Al-Khalili, L., M. Forsgren, et al. (2005). “Enhanced insulin-stimulated glycogen synthesis in response to insulin, metformin or rosiglitazone is associated with increased mRNA expression of GLUT4 and peroxisomal proliferator activator receptor gamma co-activator 1.” Diabetologia 48(6): 1173-9.
Arosio, M., S. Porretti, et al. (2003). “Elevated circulating somatostatin levels in acromegaly.” J Endocrinol Invest 26(6): 499-502.
Astaneie, F., M. Afshari, et al. (2005). “Total antioxidant capacity and levels of epidermal growth factor and nitric oxide in blood and saliva of insulin-dependent diabetic patients.” Arch Med Res 36(4): 376-81.
Authier, F., M. Kouach, et al. (2005). “Endosomal proteolysis of insulin-like growth factor-I at its C-terminal D-domain by cathepsin B.” FEBS Lett 579(20): 4309-16.
Baranowska, B., E. Wolinska-Witort, et al. (2005). “Plasma orexin A, orexin B, leptin, neuropeptide Y (NPY) and insulin in obese women.” Neuro Endocrinol Lett 26(4): 293-6.
Baratta, R., R. Di Paola, et al. (2003). “Evidence for genetic epistasis in human insulin resistance: the combined effect of PC-1 (K121Q) and PPARgamma2 (P12A) polymorphisms.” J Mol Med 81(11): 718-23.
Beale, E. G., R. E. Hammer, et al. (2004). “Disregulated glyceroneogenesis: PCK1 as a candidate Diabetes and obesity gene.” Trends Endocrinol Metab 15(3): 129-35.
Bell, C. G., D. Meyre, et al. (2005). “Association of melanin-concentrating hormone receptor 1 5′ polymorphism with early-onset extreme obesity.” Diabetes 54(10): 3049-55.
Beranek, M., K. Kankova, et al. (2002). “Polymorphisms in the von Willebrand factor gene are not associated with proliferative retinopathy in non-insulin-dependent Diabetes Mellitus.” Ophthalmic Res 34(5): 327-30.
Bernassola, F., M. Federici, et al. (2002). “Role of transglutaminase 2 in glucose tolerance: knockout mice studies and a putative mutation in a MODY patient.” Faseb J 16(11): 1371-8.
Bertherat, J., F. Tenenbaum, et al. (2003). “Somatostatin receptors 2 and 5 are the major somatostatin receptors in insulinomas: an in vivo and in vitro study.” J Clin Endocrinol Metab 88(11): 5353-60.
Bicknell, R, Vallee, B. L. (1989). “Angiogenin stimulates endothelial cell prostacyclin secretion by activation of phospholipase A2.” Proc Natl Acad Sci USA. 86(5): 1573-7.
Bluher, M., R. Unger, et al. (2002). “Relation between glycaemic control, hyperinsulinaemia and plasma concentrations of soluble adhesion molecules in patients with impaired glucose tolerance or Type II Diabetes.” Diabetologia 45(2): 210-6.
Boucher, J., B. Masri, et al. (2005). “Apelin, a newly identified adipokine up-regulated by insulin and obesity.” Endocrinology 146(4): 1764-71.
Boutin, P., C. Dina, et al. (2003). “GAD2 on chromosome 10p12 is a candidate gene for human obesity.” PLoS Biol 1(3): E68.

Brackenridge, A., E. R. Pearson, et al. (2006). “Contrasting insulin sensitivity of endogenous glucose production rate in subjects with hepatocyte nuclear factor-1beta and -1alpha mutations.” Diabetes 55(2): 405 -11.
Breiman, L. (2001), Random Forests, Machine Learning 45(1), 5-32.
Breiman, Friedman, Olshen, and Stone. (1984) Classification and Regression Trees. Wadsworth Press.
Bruun, J. M., C. Verdich, et al. (2003). “Association between measures of insulin sensitivity and circulating levels of interleukin-8, interleukin-6 and tumor necrosis factor-alpha. Effect of weight loss in obese men.” Eur J Endocrinol 148(5): 535-42.
Bullmann, C., J. Kotzka, et al. (2002). “Identification of a novel mutation in the arginine vasopressin-neurophysin II gene in familial central Diabetes insipidus.” Exp Clin Endocrinol Diabetes 110(3): 134-7.
Burgmann, H., Hollenstein, U., Maca, T., Zedwitz-Liebenstein, K., Thalhammer, F., Koppensteiner, R., Ehringer, H., Graninger, W. J. (1996) “Increased serum laminin and angiogenin concentrations in patients with peripheral arterial occlusive disease.” Clin Pathol. 49(6): 508-10.
Carroll, J. S., E. W. Seely, et al. (2001). “Digitalis-like factor response to hyperinsulinemia accompanying a euglycemic hyperinsulinemic clamp or oral glucose tolerance test.” Life Sci 69(7): 829-37.
Chang, F. Y, Shaio, M. F. (1995) “Decreased cell-mediated immunity in patients with non-insulin-dependent diabetes mellitus.” Diabetes Res Clin Pract. 28(2):137-46.
Chen, M. B., A. J. McAinch, et al. (2005). “Impaired activation of AMP-kinase and fatty acid oxidation by globular adiponectin in cultured human skeletal muscle of obese type 2 diabetics.” J Clin Endocrinol Metab 90(6): 3665-72.
Chen, M. P., F. M. Chung, et al. (2006). “Elevated plasma level of visfatin/pre-B cell colony-enhancing factor in patients with type 2 Diabetes Mellitus.” J Clin Endocrinol Metab 91(1): 295-9.
Cho, Y. M., M. D. Ritchie, et al. (2004). “Multifactor-dimensionality reduction shows a two-locus interaction associated with Type 2 Diabetes Mellitus.” Diabetologia 47(3): 549-54.
Chouchane, L., J. Danguir, et al. (2001). “Genetic variation in the stress protein hsp70-2 gene is highly associated with obesity.” Int J Obes Relat Metab Disord 25(4): 462-6.
Cristianini, N. and Shawe-Taylor, J (2000) An Introduction to Support Vector Machines and other kernel-based learning methods. Cambridge University Press
Clemmons, D. R., M. Sleevi, et al. (2005). “Recombinant, nonglycosylated human insulin-like growth factor-binding protein-3 (IGFBP-3) is degraded preferentially after administration to type II diabetics, resulting in increased endogenous glycosylated IGFBP-3.” J Clin Endocrinol Metab 90(12): 6561-8.
Collin, G. B., J. D. Marshall, et al. (2002). “Mutations in ALMS 1 cause obesity, type 2 Diabetes and neurosensory degeneration in Alstrom syndrome.” Nat Genet 31(1): 74-8.
Cook, N. S. (2007) “Use and misuse of the receiver operating characteristic curve in risk prediction.” Circulation 115(7): 928-35.
Cosson, E., A. F. Bringuier, et al. (2005). “Fas/Fas-Ligand pathway is impaired in patients with type 2 Diabetes. Influence of hypertension and insulin resistance.” Diabetes Metab 31(1): 47-54.
Cryns, K., T. A. Sivakumaran, et al. (2003). “Mutational spectrum of the WFS1 gene in Wolfram syndrome, nonsyndromic hearing impairment, Diabetes Mellitus, and psychiatric disease.” Hum Mutat 22(4): 275-87.
Cummings, D. E., J. Q. Purnell, et al. (2001). “A preprandial rise in plasma ghrelin levels suggests a role in meal initiation in humans.” Diabetes 50(8): 1714-9.
Dantz, D., J. Bewersdorf, et al. (2002). “Vascular endothelial growth factor: a novel endocrine defensive response to hypoglycemia.” J Clin Endocrinol Metab 87(2): 835-40.
Delplanque, J., F. Vasseur, et al. (2002). “Mutation screening of the urocortin gene: identification of new single nucleotide polymorphisms and association studies with obesity in French Caucasians.” J Clin Endocrinol Metab 87(2): 867-9.
Dempfle, A., A. Hinney, et al. (2004). “Large quantitative effect of melanocortin-4 receptor gene mutations on body mass index.” J Med Genet 41(10): 795-800.
Du, K., S. Herzig, et al. (2003). “TRB3: a tribbles homolog that inhibits Akt/PKB activation by insulin in liver.” Science 300(5625): 1574-7.
Dzienis-Straczkowska, S., M. Straczkowski, et al. (2003). “Soluble tumor necrosis factor-alpha receptors in young obese subjects with normal and impaired glucose tolerance.” Diabetes Care 26(3): 875-80.
Esposito, K., R. Marfella, et al. (2004). “Plasma interleukin-18 concentrations are elevated in type 2 Diabetes.” Diabetes Care 27(1): 272.
Faraj, M., P. J. Havel, et al. (2003). “Plasma acylation-stimulating protein, adiponectin, leptin, and ghrelin before and after weight loss induced by gastric bypass surgery in morbidly obese subjects.” J Clin Endocrinol Metab 88(4): 1594-602.
Feng, X., Z. Guo, et al. (2002). “Identification of a negative response element in the human inducible nitric-oxide synthase (hiNOS) promoter: The role of NF-kappa B-repressing factor (NRF) in basal repression of the hiNOS gene.” Proc Natl Acad Sci USA 99(22): 14212-7.
Feng, Y., Q. Wang, et al. (2005). “SGK1-mediated fibronectin formation in diabetic nephropathy.” Cell Physiol Biochem 16(4-6): 237-44.
Fernandez-Real, J. M., M. Broch, et al. (2003). “CD14 monocyte receptor, involved in the inflammatory cascade, and insulin sensitivity.” J Clin Endocrinol Metab 88(4): 1780-4.
Fernandez-Real, J. M., M. Pugeat, et al. (2002). “Serum corticosteroid-binding globulin concentration and insulin resistance syndrome: a population study.” J Clin Endocrinol Metab 87(10): 4686-90.
Fontaine, C., G. Dubois, et al. (2003). “The orphan nuclear receptor Rev-Erbalpha is a peroxisome proliferator-activated receptor (PPAR) gamma target gene and promotes PPARgamma-induced adipocyte differentiation.” J Biol Chem 278(39): 37672-80.
Forbes, J. M., S. R. Thorpe, et al. (2005). “Modulation of soluble receptor for advanced glycation end products by angiotensin-converting enzyme-1 inhibition in diabetic nephropathy.” J Am Soc Nephrol 16(8): 2363-72.
Fukui, M., Y. Kitagawa, et al. (2005). “Association between alcohol consumption and serum dehydroepiandrosterone sulphate concentration in men with Type 2 Diabetes: a link to decreased cardiovascular risk.” Diabet Med 22(10): 1446-50.
Gavrila, A., J. L. Chan, et al. (2005). “Circulating melanin-concentrating hormone, agouti-related protein, and alpha-melanocyte-stimulating hormone levels in relation to body composition: alterations in response to food deprivation and recombinant human leptin administration.” J Clin Endocrinol Metab 90(2): 1047-54.
George, S., J. J. Rochford, et al. (2004). “A family with severe insulin resistance and Diabetes due to a mutation in AKT2.” Science 304(5675): 1325-8.
Ghosh, S., E. R. Hauser, et al. (1998). “A large sample of finnish diabetic sib-pairs reveals no evidence for a non-insulin-dependent Diabetes Mellitus susceptibility locus at 2qter.” J Clin Invest 102(4): 704-9.
Ghosh, S., R. M. Watanabe, et al. (1999). “Type 2 Diabetes: evidence for linkage on chromosome 20 in 716 Finnish affected sib pairs.” Proc Natl Acad Sci USA 96(5): 2198-203.
Gonzalez, M., C. Flores, et al. (2004). “Cell signalling-mediating insulin increase of mRNA expression for cationic amino acid transporters-1 and -2 and membrane hyperpolarization in human umbilical vein endothelial cells.” Pflugers Arch 448(4): 383-94.
Gouni-Berthold, I., E. Giannakidou, et al. (2004). “Association between the PPARalpha L162V polymorphism, plasma lipoprotein levels, and atherosclerotic disease in patients with Diabetes Mellitus type 2 and in nondiabetic controls.” Am Heart J 147(6): 1117-24.
Grant, S. F., G. Thorleifsson, et al. (2006). “Variant of transcription factor 7-like 2 (TCF7L2) gene confers risk of type 2 Diabetes.” Nat Genet 38(3): 320-3.
Gunton, J. E., R. N. Kulkarni, et al. (2005). “Loss of ARNT/HIF1 beta mediates altered gene expression and pancreatic-islet dysfunction in human type 2 Diabetes.” Cell 122(3): 337-49.
Gylvin, T., R. Nolsoe, et al. (2004). “Mutation analysis of suppressor of cytokine signalling 3, a candidate gene in Type 1 Diabetes and insulin sensitivity.” Diabetologia 47(7): 1273-7.
Haap, M., C. Thamer, et al. (2002). “Metabolic characterization of a woman homozygous for the Ser113Leu missense mutation in carnitine palmitoyl transferase II.” J Clin Endocrinol Metab 87(5): 2139-43.
Hamid, Y. H., S. A. Urhammer, et al. (2004). “Variation in the interleukin-6 receptor gene associates with type 2 Diabetes in Danish whites.” Diabetes 53(12): 3342-5.
Hansen, S. K., C. S. Rose, et al. (2005). “Variation near the hepatocyte nuclear factor (HNF)-4alpha gene associates with type 2 Diabetes in the Danish population.” Diabetologia 48(3): 452-8.
Hara, K., M. Horikoshi, et al. (2005). “Absence of an association between the polymorphisms in the genes encoding adiponectin receptors and type 2 Diabetes.” Diabetologia 48(7): 1307-14.
Hardin, J. and Hilbe, J. (2001). Generalized Linear Models and Extensions. Stata Press. Texas.
Hart, L. M., R. P. Stolk, et al. (1999). “Prevalence of variants in candidate genes for type 2 Diabetes Mellitus in The Netherlands: the Rotterdam study and the Hoorn study.” J Clin Endocrinol Metab 84(3): 1002-6.
Hasegawa, G., M. Ohta, et al. (2005). “Increased serum resistin levels in patients with type 2 Diabetes are not linked with markers of insulin resistance and adiposity.” Acta Diabetol 42(2): 104-9.
Hauache, O. M., A. F. Reis, et al. (2005). “Estimation of Diabetes risk in Brazilian population by typing for polymorphisms in HLA-DR-DQ, INS and CTLA-4 genes.” Dis Markers 21(3): 139-45.
He, Z. H. and L. H. Ma (2005). “The aerobic fitness (V02 peak) and alpha-fibrinogen genetic polymorphism in obese and non-obese Chinese boys.” Int J Sports Med 26(4): 253-7.
Herlihy, O. M., B. A. Barrow, et al. (2002). “Hyperglycaemic siblings of Type II (non-insulin-dependent) diabetic patients have increased PAI-1, central obesity and insulin resistance compared with their paired normoglycaemic sibling.” Diabetologia 45(5): 635-41.
Hernandez, C., E. Carrasco, et al. (2005). “Somatostatin molecular variants in the vitreous fluid: a comparative study between diabetic patients with proliferative diabetic retinopathy and nondiabetic control subjects.” Diabetes Care 28(8): 1941-7.
Hirai, S., M. Yamanaka, et al. (2005). “Activin A inhibits differentiation of 3T3-L1 preadipocyte.” Mol Cell Endocrinol 232(1-2): 21-6.
Horike, N., H. Takemori, et al. (2003). “Adipose-specific expression, phosphorylation of Ser794 in insulin receptor substrate-1, and activation in diabetic animals of salt-inducible kinase-2.” J Biol Chem 278(20): 18440-7.
Hotamisligil, G. S. and B. M. Spiegelman (1994). “Tumor necrosis factor alpha: a key component of the obesity-Diabetes link.” Diabetes 43(11): 1271-8.
Hristova, M. and L. Aloe (2006). “Metabolic syndrome—neurotrophic hypothesis.” Med Hypotheses 66(3): 545-9.
Humpert, P. M., S. Kopf, et al. (2006). “Plasma sRAGE is independently associated with urinary albumin excretion in type 2 Diabetes.” Diabetes Care 29(5): 1111-3.
Ide, A., E. Kawasaki, et al. (2002). “Genetic association between interleukin-10 gene promoter region polymorphisms and type 1 Diabetes age-at-onset.” Hum Immunol 63(8): 690-5.
Illig, T., F. Bongardt, et al. (2004). “Significant association of the interleukin-6 gene polymorphisms C-174G and A-598G with type 2 Diabetes.” J Clin Endocrinol Metab 89(10): 5053-8.
Itoh, Y., Y. Kawamata, et al. (2003). “Free fatty acids regulate insulin secretion from pancreatic beta cells through GPR40.” Nature 422(6928): 173-6.
Jayagopal, V., E. S. Kilpatrick, et al. (2004). “The biological variation of sex hormone-binding globulin in type 2 Diabetes: implications for sex hormone-binding globulin as a surrogate marker of insulin resistance.” Diabetes Care 27(1): 278-80.
Jinchuan, Y., W. Zonggui, et al. (2004). “Upregulation of CD40-CD40 ligand system in patients with Diabetes Mellitus.” Clin Chim Acta 339(1-2): 85-90.
Johannesen, J., A. Pie, et al. (2001). “Linkage of the human inducible nitric oxide synthase gene to type 1 Diabetes.” J Clin Endocrinol Metab 86(6): 2792-6.
Johansen, T., C. Laurino, et al. (2005). “Reduction of adiposity with prolonged growth hormone treatment in old obese rats: effects on glucose handling and early insulin signaling.” Growth Horm IGF Res 15(1): 55-63.
Kalina, A., C. Szalai, et al. (2002). “Association of plasma lipid levels with apolipoprotein E polymorphism in Type 2 Diabetes.” Diabetes Res Clin Pract 56(1): 63-8.
Kalmar, T., I. Seres, et al. (2005). “Correlation between the activities of lipoprotein lipase and paraoxonase in type 2 Diabetes Mellitus.” Diabetes Metab 31(6): 574-80.
Kaneto, H., T. A. Matsuoka, et al. (2005). “A crucial role of MafA as a novel therapeutic target for Diabetes.” J Biol Chem 280(15): 15047-52.
Kim, H. S., S. Yumkham, et al. (2005). “C-terminal part of AgRP stimulates insulin secretion through calcium release in pancreatic beta Rin5mf cells.” Neuropeptides 39(4): 385-93.
Kim, J. H., H. D. Shin, et al. (2005). “Peroxisome proliferator-activated receptor gamma coactivator 1 alpha promoter polymorphisms are associated with early-onset type 2 Diabetes Mellitus in the Korean population.” Diabetologia 48(7): 1323-30.
Kim, M. J., M. Maachi, et al. (2006). “Increased adiponectin receptor-1 expression in adipose tissue of impaired glucose-tolerant obese subjects during weight loss.” Eur J Endocrinol 155(1): 161-5.
Koka, V., W. Wang, et al. (2006). “Advanced glycation end products activate a chymase-dependent angiotensin II-generating pathway in diabetic complications.” Circulation 113(10): 1353-60.
Konheim, Y. L. and J. K. Wolford (2003). “Association of a promoter variant in the inducible cyclooxygenase-2 gene (PTGS2) with type 2 Diabetes Mellitus in Pima Indians.” Hum Genet 113(5): 377-81.
Koo, S. H., L. Flechner, et al. (2005). “The CREB coactivator TORC2 is a key regulator of fasting glucose metabolism.” Nature 437(7062): 1109-11.
Kotnik, P., J. Nielsen, et al. (2005). “Altered expression of COX-1, COX-2, and mPGES in rats with nephrogenic and central Diabetes insipidus.” Am J Physiol Renal Physiol 288(5): F1053-68.
Lakka, T. A., H. M. Lakka, et al. (2005). “Effect of exercise training on plasma levels of C-reactive protein in healthy adults: the HERITAGE Family Study.” Eur Heart J 26(19): 2018-25.
Laukkanen, O., J. Lindstrom, et al. (2005). “Polymorphisms in the SLC2A2 (GLUT2) gene are associated with the conversion from impaired glucose tolerance to type 2 Diabetes: the Finnish Diabetes Prevention Study.” Diabetes 54(7): 2256-60.
Laukkanen, O., J. Pihlajamaki, et al. (2004). “Polymorphisms of the SUR1 (ABCC8) and Kir6.2 (KCNJ11) genes predict the conversion from impaired glucose tolerance to type 2 Diabetes. The Finnish Diabetes Prevention Study.” J Clin Endocrinol Metab 89(12): 6286-90.
Lee, S. W., K. E. Song, et al. (2005). “Alterations in peripheral blood levels of TIMP-1, MMP-2, and MMP-9 in patients with type-2 Diabetes.” Diabetes Res Clin Pract 69(2): 175-9.
Legakis, I., T. Mantzouridis, et al. (2005). “Positive correlation of galanin with glucose in type 2 Diabetes.” Diabetes Care 28(3): 759-60.
Lewitt, M. S., K. Hall, et al. (2005). “Altered response of insulin-like growth factor-binding protein 1 to nutritional deprivation in type 2 Diabetes Mellitus.” Metabolism 54(3): 275-80.
Lim, H. S., A. D. Blann, et al. (2004). “Soluble CD40 ligand, soluble P-selectin, interleukin-6, and tissue factor in Diabetes Mellitus: relationships to cardiovascular disease and risk factor intervention.” Circulation 109(21): 2524-8.
Lindgren, C. M., M. M. Mahtani, et al. (2002). “Genomewide search for type 2 Diabetes Mellitus susceptibility loci in Finnish families: the Botnia study.” Am J Hum Genet 70(2): 509-16.
Lo, J. C., X. Zhao, et al. (2006). “The association of genetic polymorphisms in sex hormone biosynthesis and action with insulin sensitivity and Diabetes Mellitus in women at midlife.” Am J Med 119(9 Suppl 1): S69-78.
Maedler, K., A. Fontana, et al. (2002). “FLIP switches Fas-mediated glucose signaling in human pancreatic beta cells from apoptosis to cell replication.” Proc Natl Acad Sci USA 99(12): 8236-41.
Magnusson, M., O. Melander, et al. (2004). “Elevated plasma levels of Nt-proBNP in patients with type 2 Diabetes without overt cardiovascular disease.” Diabetes Care 27(8): 1929-35.
Malamitsi-Puchner, A., Sarandakou, A., Dafogianni, C., Tziotis, J., Bartsocas, C. S. (1998) “Serum angiogenin levels in children and adolescents with insulin-dependent diabetes mellitus.” Pediatr Res. 43(6): 798-800.
Mallone, R. and P. C. Perin (2006). “Anti-CD38 autoantibodies in type? Diabetes.” Diabetes Metab Res Rev 22(4): 284-94.
Mannucci, E., L. Pala, et al. (2005). “Hyperglycaemia increases dipeptidyl peptidase IV activity in Diabetes Mellitus.” Diabetologia 48(6): 1168-72.
Marchal-Victorion, S., N. Vionnet, et al. (2002). “Genetic, pharmacological and functional analysis of cholecystokinin-1 and cholecystokinin-2 receptor polymorphism in type 2 Diabetes and obese patients.” Pharmacogenetics 12(1): 23-30.
Marculescu, R., G. Endler, et al. (2002). “Interleukin-1 receptor antagonist genotype is associated with coronary atherosclerosis in patients with type 2 Diabetes.” Diabetes 51(12): 3582-5.
Marques, R. G., M. J. Fontaine, et al. (2004). “C-peptide: much more than a byproduct of insulin biosynthesis.” Pancreas 29(3): 231-8.
Marselli, L., L. Trincavelli, et al. (2004). “The role of peripheral benzodiazepine receptors on the function and survival of isolated human pancreatic islets.” Eur J Endocrinol 151(2): 207-14.

Mathieu, C., C. Gysemans, et al. (2005). “Vitamin D and Diabetes.” Diabetologia 48(7): 1247-57.
McCullagh P. and Nelder, J. A. (1989) Generalized Linear Models. London: Chapman and Hall.
Miele, C., A. Riboulet, et al. (2003). “Human glycated albumin affects glucose metabolism in L6 skeletal muscle cells by impairing insulin-induced insulin receptor substrate (IRS) signaling through a protein kinase C alpha-mediated mechanism.” J Biol Chem 278(48): 47376-87.
Migdalis, L., T. Thomaides, et al. (2001). “Changes of gastric emptying rate and gastrin levels are early indicators of autonomic neuropathy in type II diabetic patients.” Clin Auton Res 11(4): 259-63.
Milewicz, A., E. Mikulski, et al. (2000). “Plasma insulin, cholecystokinin, galanin, neuropeptide Y and leptin levels in obese women with and without type 2 Diabetes Mellitus.” Int J Obes Relat Metab Disord 24 Suppl 2: S152-3.
Minn, A. H., C. Hafele, et al. (2005). “Thioredoxin-interacting protein is stimulated by glucose through a carbohydrate response element and induces beta-cell apoptosis.” Endocrinology 146(5): 2397-405.
Mironczuk, K., A. Okruszko, et al. (2005). “Interleukin 18 and sICAM-1 serum levels in families with type 1 Diabetes Mellitus.” Rocz Akad Med Bialymst 50: 151-4.
Mitamura, Y., A. Tashimo, et al. (2002). “Vitreous levels of placenta growth factor and vascular endothelial growth factor in patients with proliferative diabetic retinopathy.” Diabetes Care 25(12): 2352.
Miyake, Y., H. Eguchi, et al. (2003). “Glucose intolerance and serum aminotransferase activities in Japanese men.” J Hepatol 38(1): 18-23.
Miyawaki, K., Y. Yamada, et al. (1999). “Glucose intolerance caused by a defect in the entero-insular axis: a study in gastric inhibitory polypeptide receptor knockout mice.” Proc Natl Acad Sci USA 96(26): 14843-7.
Mohlke, K. L., A. D. Skol, et al. (2005). “Evaluation of SLC2A10 (GLUT10) as a candidate gene for type 2 Diabetes and related traits in Finns.” Mol Genet Metab 85(4): 323-7.
Mustafa, S., T. Vukovich, et al. (2004). “Haptoglobin phenotype and gestational Diabetes.” Diabetes Care 27(9): 2103-7.
Nair, S., Y. H. Lee, et al. (2004). “11beta-Hydroxysteroid dehydrogenase Type 1: genetic polymorphisms are associated with Type 2 Diabetes in Pima Indians independently of obesity and expression in adipocyte and muscle.” Diabetologia 47(6): 1088-95.
Nakagawa, T., H. Furuta, et al. (2005). “Molecular scanning of the betacellulin gene for mutations in type 2 diabetic patients.” Diabetes Res Clin Pract 68(3): 188-92.
Nomura, S., A. Shouzu, et al. (2005). “5-HT2A receptor antagonist increases circulating adiponectin in patients with type 2 Diabetes.” Blood Coagul Fibrinolysis 16(6): 423-8.
Noushmehr, H., E. D'Amico, et al. (2005). “Fatty acid translocase (FAT/CD36) is localized on insulin-containing granules in human pancreatic beta-cells and mediates fatty acid effects on insulin secretion.” Diabetes 54(2): 472-81.
O'Connell, T. and D. R. Clemmons (2002). “IGF-I/IGF-binding protein-3 combination improves insulin resistance by GH-dependent and independent mechanisms.” J Clin Endocrinol Metab 87(9): 4356-60.
Ohki-Hamazaki, H., K. Watase, et al. (1997). “Mice lacking bombesin receptor subtype-3 develop metabolic defects and obesity.” Nature 390(6656): 165-9.
Okazawa, K., Y. Yoshimasa, et al. (2005). “The haplotypes of the IRS-2 gene affect insulin sensitivity in Japanese patients with type 2 Diabetes.” Diabetes Res Clin Pract 68(1): 39-48.
Ozaki, H., Hayashi, H., Oshima, K. (1996) “Angiogenin levels in the vitreous from patients with proliferative diabetic retinopathy.” Ophthalmic Res. 28(6):356-60.
Pagliarini, D. J., S. E. Wiley, et al. (2005). “Involvement of a mitochondrial phosphatase in the regulation of ATP production and insulin secretion in pancreatic beta cells.” Mol Cell 19(2): 197-207.
Patti, M. E., A. J. Butte, et al. (2003). “Coordinated reduction of genes of oxidative metabolism in humans with insulin resistance and Diabetes: Potential role of PGC1 and NRF1.” Proc Natl Acad Sci USA 100(14): 8466-71.
Pepe, R. S., Janes, H., Longton, G., Leisenring, W., Newcomb, P. (2004) “Limitations of the odds ratio in gauging the performance of a diagnostic, prognostic, or screening marker.” Am J Epidemiol. 159(9):882-90.
Pereira, F. O., Frode, T. S., Medeiros, Y. S. (2006) “Evaluation of tumour necrosis factor alpha, interleukin-2 soluble receptor, nitric oxide metabolites, and lipids as inflammatory markers in type 2 diabetes mellitus.” Mediators Inflamm. 2006(1):39062.
Repaske, D. R., J. A. Phillips, 3rd, et al. (1990). “Molecular analysis of autosomal dominant neurohypophyseal Diabetes insipidus.” J Clin Endocrinol Metab 70(3): 752-7.
Ripley, B. D. (1996) Pattern Recognition and Neural Networks. Cambridge University Press.
Rodgers, J. T., C. Lerin, et al. (2005). “Nutrient control of glucose homeostasis through a complex of PGC-1alpha and SIRT1.” Nature 434(7029): 113-8.
Rose, C. S., J. Ek, et al. (2005). “A-30G>A polymorphism of the beta-cell-specific glucokinase promoter associates with hyperglycemia in the general population of whites.” Diabetes 54(10): 3026-31.
Rosmond, R., M. Chagnon, et al. (2003). “Increased abdominal obesity, insulin and glucose levels in nondiabetic subjects with a T29C polymorphism of the transforming growth factor-betal gene.” Horm Res 59(4): 191-4.
Roth, S. M., M. A. Schrager, et al. (2002). “IGF2 genotype and obesity in men and women across the adult age span.” Int J Obes Relat Metab Disord 26(4): 585-7.
Rudofsky, G., Jr., P. Reismann, et al. (2004). “Reduction of postprandial hyperglycemia in patients with type 2 Diabetes reduces NF-kappaB activation in PBMCs.” Horm Metab Res 36(9): 630-8.
Salopuro, T., J. Lindstrom, et al. (2004). “Common variants in beta2- and beta3-adrenergic receptor genes and uncoupling protein 1 as predictors of the risk for type 2 Diabetes and body weight changes. The Finnish Diabetes Prevention Study.” Clin Genet 66(4): 365-7.
Sano, H., S. C. Liu, et al. (2002). “Insulin receptor substrate 4 associates with the protein IRAS.” J Biol Chem 277(22): 19439-47.
Sauvaget, D., V. Chauffeton, et al. (2004). “In vitro transcriptional induction of the human apolipoprotein A-II gene by glucose.” Diabetes 53(3): 672-8.
Saxena, S. K., Rybak, S. M., Davey, R. T. Jr, Youle, R. J., Ackerman, E. J. 1992 “Angiogenin is a cytotoxic, tRNA-specific ribonuclease in the RNase A superfamily.” J Biol Chem. 267(30): 21982-6.
Scassa, M. E., A. S. Guberman, et al. (2004). “Hepatic nuclear factor 3 and nuclear factor 1 regulate 5-aminolevulinate synthase gene expression and are involved in insulin repression.” J Biol Chem 279(27): 28082-92.
Schlosser, M., J. P. Banga, et al. (2005). “Dynamic changes of GAD65 autoantibody epitope specificities in individuals at risk of developing type 1 Diabetes.” Diabetologia 48(5): 922-30.
Several (2006). “Nature Medicine Special Issue on Metabolic Syndrome.” Nature Medicine 12(1): 26-80.
Several (2006). “Third Annual World Congress on the Insulin Resistance Syndrome.” Diabetes Care 29: 2165-2174.
Shaat, N., M. Ekelund, et al. (2005). “Association of the E23K polymorphism in the KCNJ11 gene with gestational Diabetes Mellitus.” Diabetologia 48(12): 2544-51.
Shapiro, R., Strydom, D. J., Olson, K. A., Vallee, B. L. (1987) “Isolation of angiogenin from normal human plasma.” Biochemistry. 26(16): 5141-6.
Shen, R., Ghosh, D.,Chinnaiyan, A., Meng, Z. 2006. Eigengene-based linear discriminant model for tumor classification using gene expression microarray data. Bioinformatics, 22(21): 2635-2642.
Shimomura, I., R. E. Hammer, et al. (1998). “Insulin resistance and Diabetes Mellitus in transgenic mice expressing nuclear SREBP-1c in adipose tissue: model for congenital generalized lipodystrophy.” Genes Dev 12(20): 3182-94.
Shumay, E., X. Song, et al. (2002). “pp60Src mediates insulin-stimulated sequestration of the beta(2)-adrenergic receptor: insulin stimulates pp60Src phosphorylation and activation.” Mol Biol Cell 13(11): 3943-54.
Siddiq, A., F. Lepretre, et al. (2005). “A synonymous coding polymorphism in the alpha2-Heremans-schmid glycoprotein gene is associated with type 2 Diabetes in French Caucasians.” Diabetes 54(8): 2477-81.
Siebert J, Reiwer-Gostomska M. (2006) “Angiogenin—new important parameter in the assessment of ischaemic heart disease?” Kardiol Pol. 64(8):899-900.
Sivitz, W. I., S. M. Wayson, et al. (2003). “Leptin and body fat in type 2 Diabetes and monodrug therapy.” J Clin Endocrinol Metab 88(4): 1543-53.
Song, K. H., S. H. Ko, et al. (2005). “Prospective study of lipoprotein(a) as a risk factor for deteriorating renal function in type 2 diabetic patients with overt proteinuria.” Diabetes Care 28(7): 1718-23.
Sosa-Pineda, B. (2004). “The gene Pax4 is an essential regulator of pancreatic beta-cell development.” Mol Cells 18(3): 289-94.
Southgate, R. J., C. R. Bruce, et al. (2005). “PGC-1alpha gene expression is down-regulated by Akt-mediated phosphorylation and nuclear exclusion of FoxO1 in insulin-stimulated skeletal muscle.” Faseb J 19(14): 2072-4.
Spranger, J., A. Kroke, et al. (2003). “Inflammatory cytokines and the risk to develop type 2 Diabetes: results of the prospective population-based European Prospective Investigation into Cancer and Nutrition (EPIC)-Potsdam Study.” Diabetes 52(3): 812-7.
Taniguchi, A., M. Fukushima, et al. (2005). “Soluble E-selectin, leptin, triglycerides, and insulin resistance in nonobese Japanese type 2 diabetic patients.” Metabolism 54(3): 376-80.
Tayebjee, M. H., H. S. Lim, et al. (2004). “Matrix metalloproteinase-9 and tissue inhibitor of metalloproteinase-1 and -2 in type 2 Diabetes: effect of 1 year's cardiovascular risk reduction therapy.” Diabetes Care 27(8): 2049-51.
Terazono, K., H. Yamamoto, et al. (1988). “A novel gene activated in regenerating islets.” J Biol Chem 263(5): 2111-4.
Theodorakis, M. J., O. Carlson, et al. (2004). “Elevated plasma glucose-dependent insulinotropic polypeptide associates with hyperinsulinemia in impaired glucose tolerance.” Diabetes Care 27(7): 1692-8.
Thomas, G. N., J. A. Critchley, et al. (2003). “Renin-angiotensin system gene polymorphisms and retinopathy in chinese patients with type 2 Diabetes.” Diabetes Care 26(5): 1643-4.
Thomas, M. K., K. M. Yao, et al. (1999). “Bridge-1, a novel PDZ-domain coactivator of E2A-mediated regulation of insulin gene transcription.” Mol Cell Biol 19(12): 8492-504.
Tohjima, T., N. Honda, et al. (1998). “Decreased activity of arachidonate 12-lipoxygenase in platelets of Japanese patients with non-insulin-dependent Diabetes Mellitus.” Metabolism 47(3): 257-63.
Tokuyama, Y., K. Matsui, et al. (2004). “Five missense mutations in glucagon-like peptide 1 receptor gene in Japanese population.” Diabetes Res Clin Pract 66(1): 63-9.
Torres, S. H., J. B. De Sanctis, et al. (2004). “Inflammation and nitric oxide production in skeletal muscle of type 2 diabetic patients.” J Endocrinol 181(3): 419-27.
Tsiavou, A., E. Hatziagelaki, et al. (2005). “Correlation between intracellular interferon-gamma (IFN-gamma) production by CD4+ and CD8+ lymphocytes and IFN-gamma gene polymorphism in patients with type 2 Diabetes Mellitus and latent autoimmune Diabetes of adults (LADA).” Cytokine 31(2): 135-41.
Tsuchida, H., M. Bjornholm, et al. (2002). “Gene expression of the p85alpha regulatory subunit of phosphatidylinositol 3-kinase in skeletal muscle from type 2 diabetic subjects.” Pflugers Arch 445(1): 25-31.
Ukkola, O., M. Chagnon, et al. (2003). “Genetic variation at the adipsin locus and response to long-term overfeeding.” Eur J Clin Nutr 57(9): 1073-8.
Ukkola, O., J. Salonen, et al. (2005). “Role of candidate genes in the lipid responses to intensified treatment in Type 2 Diabetes.” J Endocrinol Invest 28(10): 871-5.
Um, S. H., F. Frigerio, et al. (2004). “Absence of S6K1 protects against age- and diet-induced obesity while enhancing insulin sensitivity.” Nature 431(7005): 200-5.
Vanttinen, M., P. Nuutila, et al. (2005). “Single nucleotide polymorphisms in the peroxisome proliferator-activated receptor delta gene are associated with skeletal muscle glucose uptake.” Diabetes 54(12): 3587-91.
Vasan, R. S. (2006) “Biomarkers of cardiovascular disease: molecular basis and practical considerations.” Circulation 113(19): 2335-62.
Venables, W. N. and Ripley, B. D. (2002) Modern Applied Statistics with S. New York: Springer.
Vettor, R., G. Milan, et al. (2005). “Review article: adipocytokines and insulin resistance.” Aliment Pharmacol Ther 22 Suppl 2: 3-10.
Wahab, N. A., L. Schaefer, et al. (2005). “Glomerular expression of thrombospondin-1, transforming growth factor beta and connective tissue growth factor at different stages of diabetic nephropathy and their interdependent roles in mesangial response to diabetic stimuli.” Diabetologia 48(12): 2650-60.
Wakabayashi, M., H. Ohi, et al. (2006). “Acquired Loss of Erythrocyte Complement Receptor Type 1 in Patients with Diabetic Nephropathy Undergoing Hemodialysis.” Nephron Exp Nephrol 104(3): e89-e95.
Wautier, M. P., E. Boulanger, et al. (2004). “AGEs, macrophage colony stimulating factor and vascular adhesion molecule blood levels are increased in patients with diabetic microangiopathy.” Thromb Haemost 91(5): 879-85.
Wei, F. Y., K. Nagashima, et al. (2005). “Cdk5-dependent regulation of glucose-stimulated insulin secretion.” Nat Med 11(10): 1104-8.
Wolford, J. K., K. A. Yeatts, et al. (2005). “Sequence variation in PPARG may underlie differential response to troglitazone.” Diabetes 54(11): 3319-25.
Wu, B., J. Takahashi, et al. (2005). “Variants of calpain-10 gene and its association with type 2 Diabetes Mellitus in a Chinese population.” Diabetes Res Clin Pract 68(2): 155-61.
Xue, Y. M., L. Zhou, et al. (2002). “Correlation between angiotensin II type 1 receptor gene polymorphism and type 2 Diabetes Mellitus complicated by hypertension.” Di Yi Jun Yi Da Xue Xue Bao 22(5): 444-6.
Yagui, K., F. Shimada, et al. (1998). “A missense mutation in the CD38 gene, a novel factor for insulin secretion: association with Type II Diabetes Mellitus in Japanese subjects and evidence of abnormal function when expressed in vitro.” Diabetologia 41(9): 1024-8.
Yang, Q., T. E. Graham, et al. (2005). “Serum retinol binding protein 4 contributes to insulin resistance in obesity and type 2 Diabetes.” Nature 436(7049): 356-62.
Yang, Y., E. K. Chung, et al. (2003). “Diversity in intrinsic strengths of the human complement system: serum C4 protein concentrations correlate with C4 gene size and polygenic variations, hemolytic activities, and body mass index.” J Immunol 171(5): 2734-45.
Ye, L., Y. Xu, et al. (2002). “[Association of polymorphism in neurogenic differentiation factor 1 gene with type 2 Diabetes].” Zhonghua Yi Xue Yi Chuan Xue Za Zhi 19(6): 484-7.
Zacharova, J., J. L. Chiasson, et al. (2005). “Leptin receptor gene variation predicts weight change in subjects with impaired glucose tolerance.” Obes Res 13(3): 501-6.
Zeggini, E., J. Parkinson, et al. (2004). “Association studies of insulin receptor substrate 1 gene (IRS 1) variants in type 2 Diabetes samples enriched for family history and early age of onset.” Diabetes 53(12): 3319-22.
Zhang, J., R. I. Holt, et al. (2005). “Plasma adiponectin concentrations are independently predicted by fat insulin sensitivity in women and by muscle insulin sensitivity in men.” Diabetes Care 28(3): 755-6.
Zhu, F., L. Ji, et al. (2002). “[The role of urotensin II gene in the genetic susceptibility to type 2 Diabetes in Chinese population].” Zhonghua Yi Xue Za Zhi 82(21): 1473-5.
Zimmermann, R., J. G. Strauss, et al. (2004). “Fat mobilization in adipose tissue is promoted by adipose triglyceride lipase.” Science 306(5700): 1383-6.

What is claimed is:

1. A method for evaluating the risk of developing a diabetic condition in a subject comprising:
a. measuring at least two biomarkers in a sample from the subject, selected from the biomarkers within the group consisting of Core Biomarkers I and Core Biomarkers II and measuring at least a third biomarker from any of the biomarkers listed in Table 2; and
b. evaluating the risk of developing a diabetic condition in the subject using the biomarker measurements.

2. The method of claim 1, wherein at least 3 of the biomarkers are selected from the biomarkers within the group consisting of Core Biomarkers I and Core Biomarkers II.

3. The method of claim 1, wherein at least 1 of the biomarkers is selected from the biomarkers within Traditional Laboratory Risk Factors.

4. The method of claim 1, wherein at least 1 of the biomarkers is selected from the biomarkers within Clinical Parameters.

5. The method of claim 1, wherein at least 1 of the biomarkers is selected from the biomarkers within Additional Biomarkers I.

6. The method of claim 1, wherein at least 1 of the biomarkers is selected from the biomarkers within Additional Biomarkers II.

7. The method of claim 1 or 2, wherein at least 2 of the biomarkers are selected from the biomarkers within Core Biomarkers I.

8. The method of claim 1 or 2, wherein at least 3 of the biomarkers are selected from the biomarkers within Core Biomarkers I.

9. The method of claim 1, further comprising measuring an at least four biomarkers selected from the biomarkers within the group consisting of Core Biomarkers I, Core Biomarkers II, Traditional Laboratory Risk Factors, Clinical Parameters, Additional Biomarkers I, and Additional Biomarkers II, wherein at least two biomarkers are selected from the biomarkers within the group consisting of Core Biomarkers I and Core Biomarkers II.

10. The method of claim 1, further comprising measuring at least five biomarkers selected from the biomarkers within the group consisting of Core Biomarkers I, Core Biomarkers II, Traditional Laboratory Risk Factors, Clinical Parameters, Additional Biomarkers I, and Additional Biomarkers II, wherein at least two biomarkers are selected from the biomarkers within the group consisting of Core Biomarkers I and Core Biomarkers II.

11. The method of claim 1, wherein the risk evaluation comprises calculating an index value using a formula incorporating the biomarker measurements.

12. The method of claim 1, wherein the risk evaluation comprises normalizing the biomarker measurements to reference values.

13. The method of claim 1, wherein one of the biomarkers is INS.

14. The method of claim 1, wherein one of the biomarkers is LEP.

15. The method of claim 1, wherein one of the biomarkers is ADIPOQ.

16. The method of claim 1, wherein one of the biomarkers is CRP.

17. The method of claim 1, wherein one of the biomarkers is FGA.

18. The method of claim 13, wherein one of the biomarkers is LEP.

19. The method of claim 13, wherein one of the biomarkers is ADIPOQ.

20. The method of claim 13, wherein one of the biomarkers is CRP.

21. The method of claim 13, wherein one of the biomarkers is FGA.

22. The method of claim 14, wherein one of the biomarkers is ADIPOQ.

23. The method of claim 14, wherein one of the biomarkers is CRP.

24. The method of claim 14, wherein one of the biomarkers is FGA.

25. The method of claim 15, wherein one of the biomarkers is CRP.

26. The method of claim 15, wherein one of the biomarkers is FGA.

27. The method of claim 15, wherein one of the biomarkers is HBA1C.

28. The method of claim 16, wherein one of the biomarkers is FGA.

29. The method of claim 16, wherein one of the biomarkers is Glucose.

30. A method of calculating an index value for use in evaluating the risk of developing a diabetic condition in a subject, comprising:
a. Measuring at least 3 biomarkers selected from the biomarkers within the group consisting of Core Biomarkers I, Core Biomarkers II, Traditional Laboratory Risk Factors, Clinical Parameters, Additional Biomarkers I, and Additional Biomarkers II, wherein at least two biomarkers are selected from the biomarkers within the group consisting of Core Biomarkers I and Core Biomarkers II in the calculation of an index value for use in evaluating the risk of developing a diabetic condition in a subject.

31. The method of claim 30, wherein at least 1 of the biomarkers is selected from the biomarkers within Traditional Laboratory Risk Factors.

32. The method of claim 30 wherein at least 1 of the biomarkers is selected from the biomarkers within Clinical Parameters.

33. A kit for calculating an index value that evaluates the risk of developing a diabetic condition in a subject comprising:
a. Reagents for measuring 3 or more biomarkers in a sample from the subject selected from the biomarkers within the group consisting of Core Biomarkers I, Core Biomarkers II, Traditional Laboratory Risk Factors, Additional Biomarkers I, and Additional Biomarkers II, wherein at least two biomarkers are selected from the biomarkers within the group consisting of Core Biomarkers I and Core Biomarkers II; and
b. Instructions for use in calculating the index value.

34. The kit of claim 33, for use with an instrument.

35. The kit of claim 33, wherein at least one of said reagents comprises a detectable label.

36. A method for evaluating the risk of developing a diabetic condition in a subject comprising:
a. measuring at least 3 biomarkers selected from the biomarkers within the group consisting of Core Biomarkers I, Core Biomarkers II, Traditional Laboratory Risk Factors, Additional Biomarkers I, and Additional Biomarkers II, wherein at least two biomarkers are selected from the biomarkers within the group consisting of Core Biomarkers I and Core Biomarkers II, and wherein accuracy of the combination of biomarkers selected is greater than the accuracy of any one of the biomarkers within the selected group, and
b. evaluating the risk of developing a diabetic condition in the subject using the biomarker measurements.

37. The method of claim 36, wherein the accuracy is measured as an increase in a positive predictive value.

38. The method of claim 36, wherein the accuracy is measured as an increase in a negative predictive value.

39. The method of claim 1, further comprising using the biomarker measurements to calculate an index value, wherein the index value is correlated with the risk of developing a diabetic condition in the subject.

40. The method of claim 11, further comprising correlating the index value to the risk of developing a diabetic condition in the subject.

41. The method of claim 3 or 4, further comprising using the biomarker measurements to calculate an index value, wherein the index value is correlated with the risk of developing a diabetic condition in the subject.

42. The method of claim 1 wherein the measurement of at least one of the biomarkers selected is unaffected by treatment of the subject with one or more therapeutic interventions.

43. The method of claim 1 wherein the measurement of at least one of the biomarkers selected is affected by treatment of the subject with one or more therapeutic interventions.

44. The method of claim 42 or 43, wherein a therapeutic intervention comprises one or more of insulin, insulin analogs, hypoglycemic agents, anti-inflammatory agents, lipid-reducing agents, calcium channel blockers, beta-adrenergic receptor blocking agents, COX-2 inhibitors, prodrugs of COX-2 inhibitors, angiotensin II antagonists, angiotensin converting enzyme (ACE) inhibitors, renin inhibitors, lipase inhibitors, amylin analogs, sodium-glucose cotransporter 2 inhibitors, dual adipose triglyceride lipase and PI3 kinase activators, antagonists of neuropeptide Y receptors, human hormone analogs, cannabinoid receptor antagonists, triple monoamine oxidase reuptake inhibitors, inhibitors of norepinephrine and dopamine reuptake, inhibitors of 11β-hydroxysteroid dehydrogenase type 1 (11b-HSD1), inhibitors of cortisol synthesis, inhibitors of gluconeogenesis, glucokinase activators, antisense inhibitors of protein tyrosine phosphatase-1B, islet neogenesis therapy, or betahistine.

45. The method of claim 1 wherein the diabetic condition is Type 2 Diabetes.

46. The method of claim 1 wherein the diabetic condition is pre-Diabetes.

47. The method of claim 19, wherein one of the biomarkers is HBA1C.

48. The method of claim 20, wherein one of the biomarkers is ADIPOQ.

49. The method of claim 25, wherein one of the biomarkers is HBA1C.

50. The method of claim 25, wherein one of the biomarkers is SBP.

51. The method of claim 28, wherein one of the biomarkers is HBA1C.

52. The method of claim 1, wherein at least 3 of the biomarkers are selected from the biomarkers within the group consisting of Core Biomarkers I, Core Biomarkers II, Traditional Laboratory Risk Factors, Clinical Parameters, and Additional Biomarkers I.

53. The method of claim 1, wherein at least 3 of the biomarkers are selected from the biomarkers within the group consisting of Core Biomarkers I, Core Biomarkers II, Traditional Laboratory Risk Factors, and Clinical Parameters.

54. The method of claim 1, wherein at least 3 of the biomarkers are selected from the biomarkers within the group consisting of Core Biomarkers I, Core Biomarkers II, Traditional Laboratory Risk Factors, Clinical Parameters, and Additional Biomarkers II.

55. A method for evaluating the risk of developing a diabetic condition in a subject comprising:
a. measuring of at least three biomarkers in a sample from the subject, wherein a first biomarker is ADIPOQ, a second biomarker is selected from the biomarkers within Core Biomarkers I, and a third biomarker is selected from the biomarkers within Core Biomarkers I or Core Biomarkers II, and
b. evaluating the risk of developing a diabetic condition in the subject using the biomarker measurements.

56. The method of claim 55, wherein the second biomarker is IGFBP1.

57. The method of claim 55, wherein the third biomarker is INS.

58. The method of claim 55, wherein at least four biomarkers are selected from the biomarkers within each of Core Biomarkers I and Core Biomarkers II.

59. In a method of evaluating the risk of developing a diabetic condition in a subject by measuring one or more of Clinical Parameters and Traditional Laboratory Risk Factors, the improvement comprising:
a. Measuring at least two biomarkers in a sample from the subject selected from the biomarkers within the group consisting of Core Biomarkers I and Core Biomarkers II; and
b. evaluating the risk of developing a diabetic condition in the subject using the biomarker measurements.

60. The method of claim 59, wherein at least 3 of the biomarkers are selected from the biomarkers within the group consisting of Core Biomarkers I and Core Biomarkers II.

61. The method of claim 59 or 60, wherein at least 2 of the biomarkers are selected from the biomarkers within Core Biomarkers I.

62. The method of claim 60, wherein at least 3 of the biomarkers are selected from the biomarkers within Core Biomarkers I.

63. The method of claim 59, further comprising measuring at least four biomarkers selected from the biomarkers within the group consisting of Core Biomarkers I, Core Biomarkers II, Traditional Laboratory Risk Factors, Clinical Parameters, Additional Biomarkers I, and Additional Biomarkers II, wherein at least two biomarkers are selected from the biomarkers within the group consisting of Core Biomarkers I and Core Biomarkers II.

64. The method of claim 59, further comprising measuring at least five biomarkers selected from the biomarkers within the group consisting of Core Biomarkers I, Core Biomarkers II, Traditional Laboratory Risk Factors, Clinical Parameters, Additional Biomarkers I, and Additional Biomarkers II, wherein at least two biomarkers are selected from the biomarkers within the group consisting of Core Biomarkers I and Core Biomarkers II.

65. The method of claim 59, wherein the risk evaluation comprises calculating an index value using a formula incorporating the biomarker measurements.

66. The method of claim 59, wherein the risk evaluation comprises normalizing the biomarker measurements to reference values.

67. The method of claim 59, wherein one of the biomarkers is INS.

68. The method of claim 59, wherein one of the biomarkers is LEP.

69. The method of claim 59, wherein one of the biomarkers is ADIPOQ.

70. The method of claim 59, wherein one of the biomarkers is CRP.

71. The method of claim 59, wherein one of the biomarkers is FGA.

72. The method of claim 67, wherein one of the biomarkers is LEP.

73. The method of claim 67, wherein one of the biomarkers is ADIPOQ.

74. The method of claim 67, wherein one of the biomarkers is CRP.

75. The method of claim 67, wherein one of the biomarkers is FGA.

76. The method of claim 68, wherein one of the biomarkers is ADIPOQ.

77. The method of claim 68, wherein one of the biomarkers is CRP.

78. The method of claim 68, wherein one of the biomarkers is FGA.

79. The method of claim 69, wherein one of the biomarkers is CRP.

80. The method of claim 69, wherein one of the biomarkers is FGA.

81. The method of claim 69, wherein one of the biomarkers is HBA1C.

82. The method of claim 70, wherein one of the biomarkers is FGA.

83. The method of claim 70, wherein one of the biomarkers is Glucose.

84. In a method of evaluating the risk of developing a diabetic condition in a subject by measuring one or more of Clinical Parameters and Traditional Laboratory Risk Factors, the improvement comprising:
a. Measuring at least two biomarkers in a sample from the subject selected from the biomarkers consisting of ADIPOQ, CRP, FGA, INS, LEP, AGER, AHSG, ANG, APOE, CD14, FTH1, IGFBP1, IL2RA, VCAM1,
VEGF, VWF; and
b. Evaluating the risk of developing a diabetic condition in the subject using the biomarker measurements.

85. A method comprising screening a population of individuals with a method according to any of claims 1, 30, 36, 55, 59 or 84.

86. The method of claim 85, further comprising compiling the results of said population screen in a data array.

87. The method of claim 85 wherein said compiled results include the evaluated risk of developing a diabetic condition.

88. The method of claim 85 wherein said compiled results include the measurement of at least one of said biomarkers.

89. In a health-related data management system comprising evaluating or tracking a health risk or condition for a subject or a population, the improvement comprising evaluating or tracking the risk of developing a diabetic condition using the data array of claim 86.
